(19) United States
(12) Patent Application Publication
(10) Pub. No.: US 2023/0265065 A1
(43) Pub. Date: Aug. 24, 2023

(54) SELECTIVE PHOSPHATASE INHIBITORS BASED ON ILLUDALIC ACID

(71) Applicant: WEST VIRGINIA UNIVERSITY BOARD OF GOVERNORS ON BEHALF OF WEST VIRGINIA UNIVERSITY, Morgantown, WV (US)

(72) Inventors: Gregory Dudley, Morgantown, WV (US); Paratchata Batsomboon, Huamark (TH); Robert Gaston, Morgantown, WV (US); Harvey Fulo, Morgantown, WV (US)

(21) Appl. No.: 18/007,296

(22) PCT Filed: Jul. 29, 2021
(86) PCT No.: PCT/US21/43713

§ 371 Date: Jan. 27, 2023

Related U.S. Application Data
(60) Provisional application No. 62/706,074, filed on Jul. 30, 2020.

(51) Int. Cl. C07D 311/92

(52) U.S. Cl. CPC C07D 311/92

(57) ABSTRACT

The present disclosure provides novel selective phosphatase inhibitor compounds based on illudalic acid. The present disclosure provides a streamlined method of synthesizing illudalic acid and a method for synthesizing phosphatase inhibitor compounds. The method of this invention provides convergent benzannulation of β-keto amides and esters, followed by a one-pot reduction/hydrolysis sequence. The concise synthetic approach provided by this invention enables rapid assembly of illudalog compounds of this invention that are potent protein tyrosine phosphatase receptor-type D (PTPRD) inhibitors.

### CROSS-REFERENCE TO RELATED APPLICATION

This utility patent application claims the benefit of co-pending U.S. Patent Application Ser. No. 62/706,074, filed on Jul. 30, 2020. The entire contents of U.S. Patent Application Ser. No. 62/706,074 are incorporated by reference into this utility patent application.

### STATEMENT REGARDING FEDERALLY SPONSORED RESEARCH OR DEVELOPMENT

Not applicable.

### BACKGROUND OF THE INVENTION

### 1. Field of the Invention

This invention provides selective phosphatase inhibitors that are analogs of illudalic acid. Namely, this invention describes the design and synthesis of molecular substances that are designed to be potent, selective, and cell-permeable LAR-PTP inhibitors based on illudalic acid. Methods of synthesis of these illudalic acid analogues are provided.

### 2. Description of the Background Art

Once considered “undruggable”,¹ protein tyrosine phosphatase (PTP) enzymes are now emerging as important (albeit still challenging) therapeutic targets for medicinal chemistry.² For example, overexpression of LAR,³ the human leukocyte common antigen-related receptor-type PTP, causes insulin resistance.⁴ The broader LAR-PTP subfamily⁵—LAR, PTPR^{δ}, and PTPR^{δ}—is linked to diabetes⁶ and obesity,⁷ addiction,⁸ various forms of cancer,⁹ and disorders of the nervous and immune systems.¹⁰ PTPs are challenging therapeutic targets in large part due to their highly conserved, positively charged catalytic domain active sites; many PTP inhibitors identified in high-throughput screening (HTS) suffer from pan-assay interference (PAIN)¹¹ and/or poor cell permeability (because the inhibitors themselves are charged).¹ One HTS effort,¹² however, identified illudalic acid,¹³ a metabolite of the toxic jack o'lantern mushroom (Clitocybe illudens), as a small molecule LAR inhibitor. A simplified analogue of illudalic acid showed promising activity in a mouse model of cocaine addiction, indicating in vivo activity for this scaffold.¹⁴ Illudalic acid offers opportunities to address the urgent need for potent and selective LAR-PIP inhibitors.¹⁵ However, illudalic acid and its unique trifunctional pharmacophore (discussed herein) are difficult to prepare. Established syntheses of illudalic acid require ≥16 chemical steps,^{16,17} and even simplified, less potent analogues based on a truncated (bicyclic) scaffold have required ≥11 linear steps.¹⁸ Innovations in chemical synthesis are needed to develop selective phosphatase inhibitors based on illudalic acid.

Reversible protein tyrosine phosphorylation, mediated by kinases (PTKs) and phosphatases (PTPs), plays a critical role in cell signaling. Once regarded as loosely regulated “signaling suppressors”, PTPs are now understood as approximately equal partners with PTKs in regulating cell processes.¹⁹ PTPs are tightly regulated, less promiscuous than initially thought, and responsible for both positive and negative signaling. The total number of known, catalytically active PTKs and PTPs expressed in human cells is similar (85 vs. 81).²⁰ Anomalous activity and/or expression of both PTKs and PTPs is linked to many common human disease states.²¹

The PTP family of enzymes includes many attractive but challenging therapeutic targets.^{1,2,22} There are 38 classical, cysteine-dependent, tyrosine-specific PTPs: 21 receptor-type, transmembrane PTPs (including the 3 LAR-PTPs) and 17 nonreceptor-type, intracellular PTPs. They share a catalytic mechanism in which a conserved arginine residue in the positively charged catalytic site facilitates binding of the phosphotyrosine-containing substrate, followed by nucleophilic attack of the catalytic cysteine residue on the phosphate.²³ The conserved, positively charged PTP catalytic site complicates therapeutic development;^{2,22} molecular design and synthesis of selective PTP inhibitors is an ambitious proposition.

In contrast to kinases,²⁴ it has proven exceedingly difficult to identify potent and selective PTP inhibitors with good bioavailability, leading some to regard this family of enzymes as “undruggable”.^{1,2} HTS campaigns have typically identified unselective, negatively charged PTP inhibitors having poor bioavailability.²⁵ However, promising inhibitors for several of the nonreceptor-type PTPs have emerged in recent years.^{2b} Allosteric inhibitors of SHP2 are in clinical trials as potential anticancer agents,^{22c,d,26} and inhibitors of PTP1B have entered clinical trials as potential treatments for diabetes and other degenerative diseases.²⁷ While receptor-type PTPs are also intriguing therapeutic targets for a host of human diseases—for example, inhibition of CD45 activity could be useful in treating autoimmune disease, as well as certain infections including Ebola and anthrax^{2a}—the development of potent, selective, and bioactive inhibitors of the receptor-type PTPs has lagged behind that of the nonreceptor-type PTPs.

The protein tyrosine phosphatase receptor-type D (PTPRD) is a therapeutic target for stimulant use disorder. Millions of Americans reported illicit stimulant use in a recent survey. Overdose deaths involving stimulants increased 42.4% from 2015 to 2016 and another 34.1% the next year to over 23,000; West Virginia reported the highest rate of stimulant-involved deaths in the nation, at 13.6 per 100,000 in 2017. Overdose deaths surged in 2020 during the pandemic, especially in conjunction with opioid abuse. The Director of NIDA recently described “an alarming increase in deaths involving the stimulant drugs methamphetamine and cocaine” and called for action in the face of this “drug addiction and overdose crisis.” A recent metareview highlights an “urgent need for effective pharmacological treatments for stimulant use disorder”; there are no FDA-approved medications.

### SUMMARY OF THE INVENTION

This invention provides potent and diverse PTPRD inhibitor compounds based on illudalic acid. As discussed herein, illudalic acid creates opportunities to design and develop selective phosphatase inhibitors based on its unique trifunctional pharmacophore and postulated two-stage, multivariable mechanism of action. The therapeutic potential of the illudalic acid pharmacophore, and specifically 7-BIA (set forth herein) and innovations in the synthesis of illudalic acid provided by this invention, create phosphatase inhibitor compounds based on illudalic acid for use in treating a patient. In mice, heterozygous Ptprd knockouts show reduced cocaine conditioned place preference and self-administration compared to wild-type. 7-BIA, a truncated analog of illudalic acid, reduces cocaine conditioned place preference and self-administration in wild-type mice, but not in the Ptprd knockout mice. No evidence of toxicity was observed in mice dosed with 7-BIA up to ˜60 mg/kg (the solubility limit). These observations establish in vivo efficacy of the illudalic acid pharmacophore. In vitro, 7-BIA inhibits PTPRD with an estimated IC₅₀ of 1-3 mM. Noted limitations of 7-BIA include its solubility, stability, and lack of broader in vivo pharmacological and toxicological analyses.

In one embodiment of this invention, a method of synthesizing illudalic acid is provided comprising carrying out each of the five steps of the following scheme:

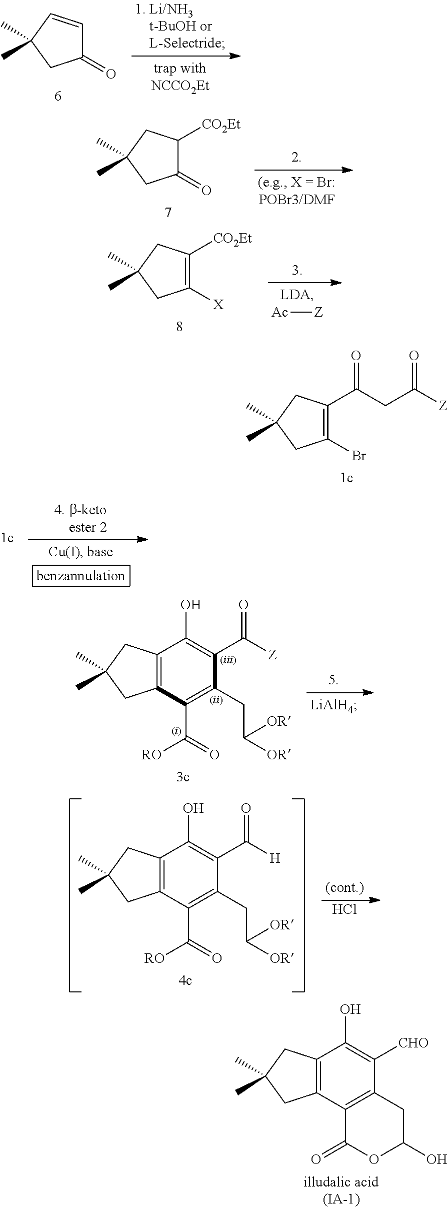

Another embodiment of this invention provides a general method of preparing illudalic acid comprising: providing benzannulation of a β-keto amide having a formula 3a

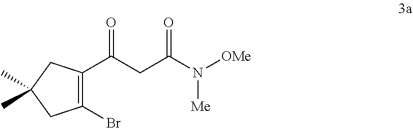

with a β-keto ester having a formula 4

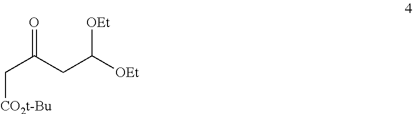 [SMILES: CCOC(CC(=O)CC(=O)OC(C)(C)C)OCC]

to produce a precursor compound having a formula 9a

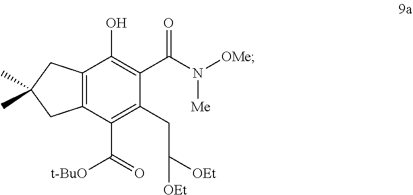

carrying out reduction of said precursor compound 9a with LiAlH₄ to form a reduced precursor compound having a formula 12

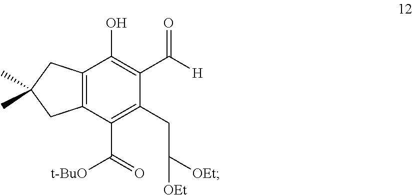

and

carrying out acid hydrolysis of said reduced precursor compound 12 to form illudalic acid having a formula IA1

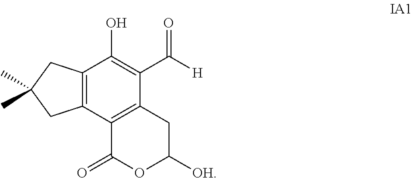

In another embodiment of this invention, this method includes subjecting a tetralin compound analogous to said reduced precursor compound 12 to two equivalents of LiAlH₄ to form a tetralin compound having a formula IA2

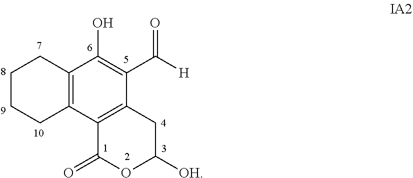 [SMILES: O=Cc1c(O)c2c(c3c1CC(O)OC3=O)CCCC2]

In another embodiment of this invention, this method includes subjecting a naphthalene compound analogous to said reduced precursor compound 12 to two equivalents of LiAlH₄ to form a naphthalene compound having a formula IA3

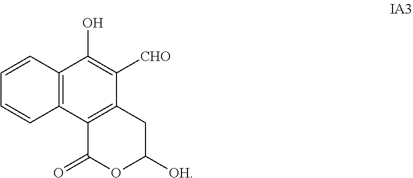 [SMILES: O=Cc1c2c(c3ccccc3c1O)C(=O)OC(O)C2]

In another embodiment of this invention, a compound is provided having a formula 13:

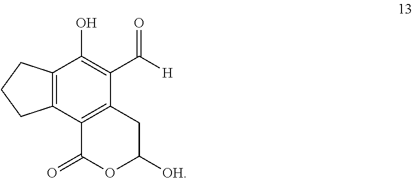 [SMILES: O=Cc1c(O)c2c(c3c1CC(O)OC3=O)CCC2]

In another embodiment of this invention, a compound is provided having a formula 14:

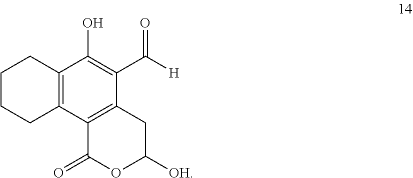 [SMILES: O=Cc1c(O)c2c(c3c1CC(O)OC3=O)CCCC2]

In another embodiment of this invention, a compound is provided having a formula 15:

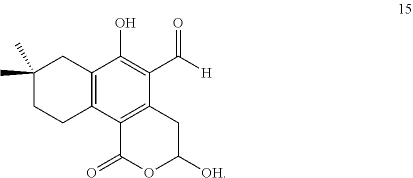

In another embodiment of this invention, a compound is provided having a formula 16:

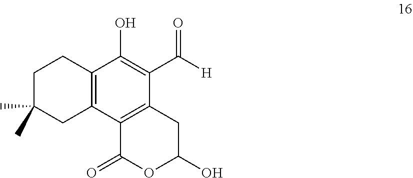

Another embodiment of this invention provides a compound that is one selected from the group of compounds consisting of IA1-6OMe, IA1-8H2, and IA1-6OMe-8H2, having one of a formula:

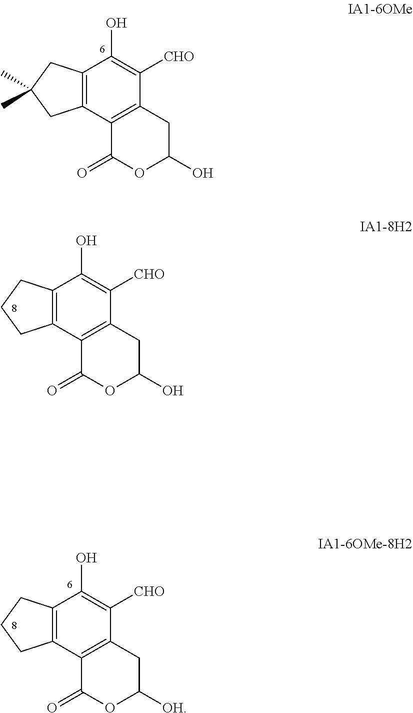

Another embodiment of this invention provides a compound that is one selected from the group of compounds consisting of IA2, IA2-8Me2, and IA2-9Me2, having one of a formula:

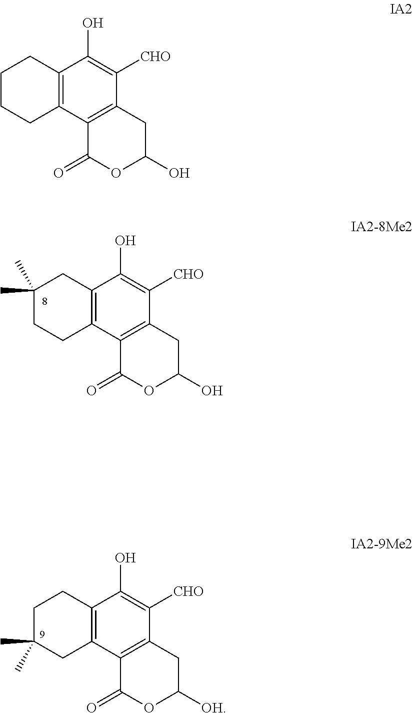

Another embodiment of this invention provides a compound that is one selected from the group of compounds consisting of IA3, IA3-6OMe, IA3-6OEt, IA3-6OC3H3, IA3-6OBn, IA3-7Me, IA3-8Me, IA3-9Me, IA3-8Br, IA3-8C2H, IA3-8C1, IA3-9C1, IA3-9tBu, IA3-9CF3, IA3-7OMe, IA3-8OMe, IA3-89OMe, IA3-89F, and IA3-6OMe89F, having one of a formula:

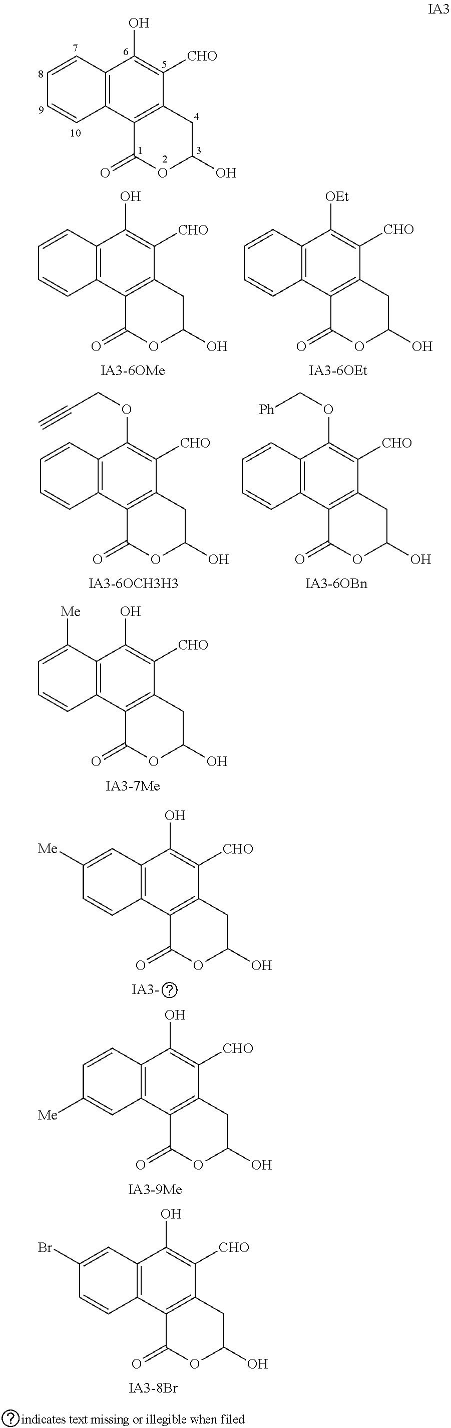

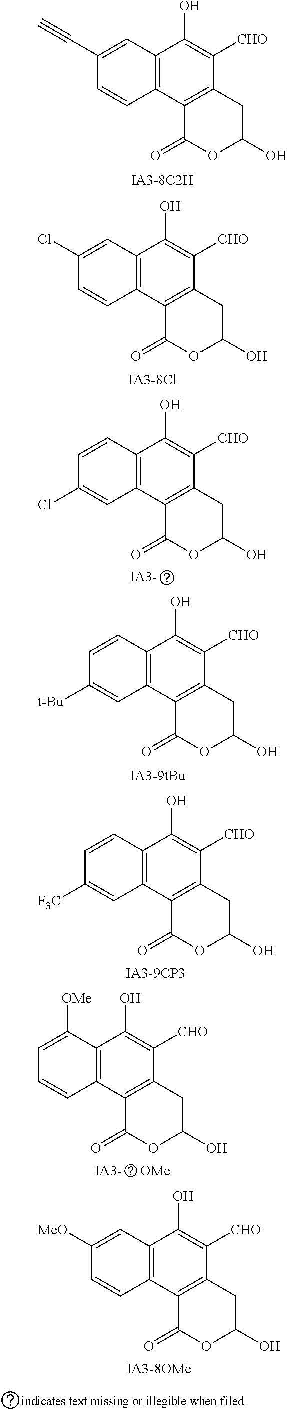

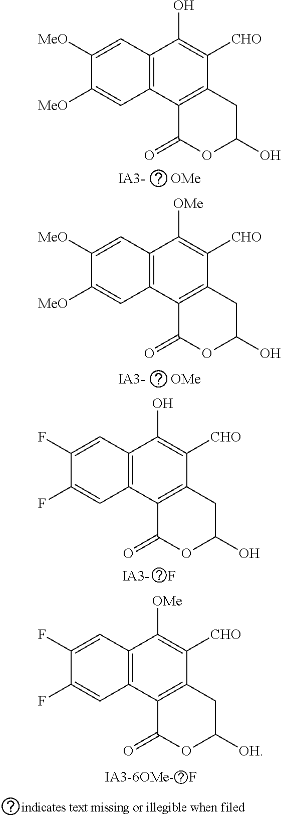 [SMILES: COc1cc2c(O)c(C=O)c3c(c2cc1OC)C(=O)OC(O)C3 ; COc1cc2c(OC)c(C=O)c3c(c2cc1OC)C(=O)OC(O)C3 ; O=Cc1c2c(c3cc(F)c(F)cc3c1O)C(=O)OC(O)C2 ; COc1c(C=O)c2c(c3cc(F)c(F)cc13)C(=O)OC(O)C2]

Another embodiment of this invention provides a compound that is one selected from the group of compounds consisting of: IA-5, IA-6, IA-7, IA-8, IA-9, and IA-10, having one of a formula:

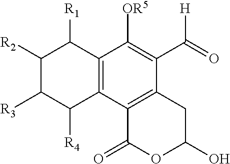

IA-5: R₁, R₂, R₃, and R₄═H, and R₅═H
IA-6: R₁, R₂, R₃, and R₄═H, and R⁵=Me (i.e. CH₃)
IA-7: R₁ and R₄═H, and R₂ and R₃=MeO, and R⁵═H
IA-8: R₁, R₃, and R₄═H, and R₂═Cl, and R⁵═H
IA-9: R₁, R₂, and R₄═H, and R₃═Cl, and R⁵═H
IA-10: R₁, R₂, and R₄═H, and R₃=t-Bu, and R⁵═H.

Another embodiment of this invention provides a compound of a formula IA-4:

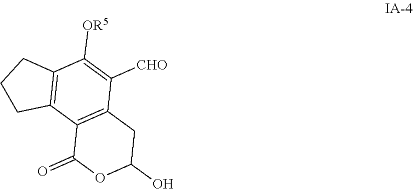

wherein R⁵ is a methyl group.

Another embodiment of this invention provides a compound that is one selected from the group of compounds consisting of IA-11-IA-30, having one of a formula:

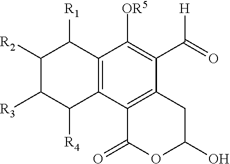

IA-12: R₃=Me, R⁵=Me, and R₂—R₄═H
IA-13: R₂=Me, R⁵═H, and R₁, R₃, and R₄═H
IA-14 R₂=Me, R⁵=Me, and R₁, R₃, and R₄═H
IA-15: R₃=Me, R⁵═H, and R₁, R₂, and R₄═H
IA-16: R₃=Me, R⁵=Me, and R₁, R₂, and R₄═H
IA-17: R₄=Me, R⁵═H, and R₁—R₃═H
IA-18: R₄=Me, R⁵=Me, and R₁—R₃═H
IA-19: R₂═Br, R⁵═H, and R₁, R₃, and R₄═H
IA-20: R₂═Br, R⁵=Me, and R₁, R₃, and R₄═H
IA-21: R₁═OMe, R⁵═H, and R₂—R₄═H
IA-22: R₁═OMe, R⁵=Me, and R₂—R₄═H
IA-23: R₂═OMe, R⁵═H, and R₁, R₃, and R₄═H
IA-24: R₂═OMe, R⁵=Me, and R₁, R₃, and R₄═H
IA-25: R₃═OMe, R⁵═H, and R₁, R₂, and R₄═H
IA-26: R₃═OMe, R⁵=Me, and R₁, R₂, and R₄═H
IA-27: R₄═OMe, R⁵═H, and R₁—R₃═H
IA-28: R₄═OMe, R⁵=Me, and R₁—R₃═H
IA-29: R₁—R₃═F, R⁵═H, and R₄═H
IA-30: R₁—R₃═F, R⁵=Me, and R₄═H.

Another embodiment of this invention provides a compound that is one selected from the group of compounds consisting of IA-31-IA-55, having one of a formula:

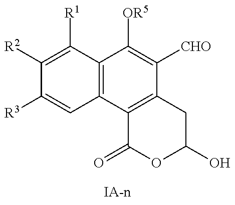

| | R¹⁻³ = H | R^{2,3} = OMe | R¹⁻³ = F | R¹ = Me | R² = Br |
|---|---|---|---|---|---|
| R⁵ = ethyl | IA-31 | IA-36 | IA-41 | IA-46 | IA-51 |
| R⁵ = butyl | IA-32 | IA-37 | IA-42 | IA-47 | IA-52 |
| R⁵ = benzyl | IA-33 | IA-38 | IA-43 | IA-48 | IA-53 |
| R⁵ = propargyl | IA-34 | IA-39 | IA-44 | IA-49 | IA-54 |
| R⁵ = CH₂CO₂Et | IA-35 | IA-40 | IA-45 | IA-50 | IA-55 |

wherein IA-36-IA-40: R₁═H; wherein IA-46-IA-50: R₂ and R₃═H; and wherein IA-51-IA 55: R₁ and R₃═H.

Another embodiment of this invention provides a compound that is one selected from the group of compounds consisting of IA-105-IA108, having one of a formula:

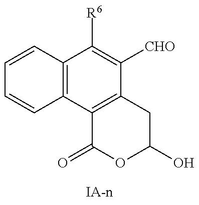

| | |
|---|---|
| IA-105: | R⁶ = methyl |
| IA-106: | R⁶ = ethynyl |
| IA-107: | R⁶ = allyl |
| IA-108: | R⁶ = phenyl |
| IA-109: | R⁶ = 2-furyl |
| IA-110: | R⁶ = H |

Another embodiment of this invention provides a compound that is one selected from the group of compounds consisting of IA3-5Me, IA3-5OMe, IA3-3H, and IA3-1Me, having one of a formula:

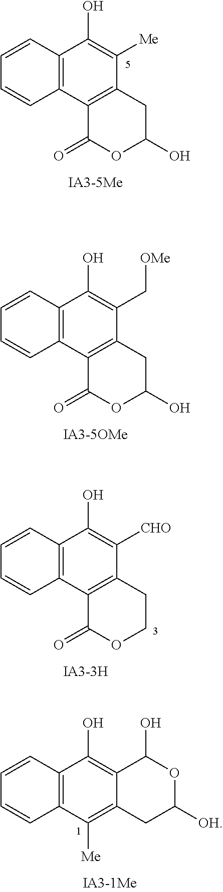 [SMILES: Cc1c2c(c3ccccc3c1O)C(=O)OC(O)C2 ; COCc1c2c(c3ccccc3c1O)C(=O)OC(O)C2 ; O=Cc1c2c(c3ccccc3c1O)C(=O)OCC2 ; Cc1c2c(c(O)c3ccccc13)C(O)OC(O)C2]

Another embodiment of this invention provides a compound that is one selected from the group of compounds consisting of IA-19, and IA-91-IA-97, having one of a formula:

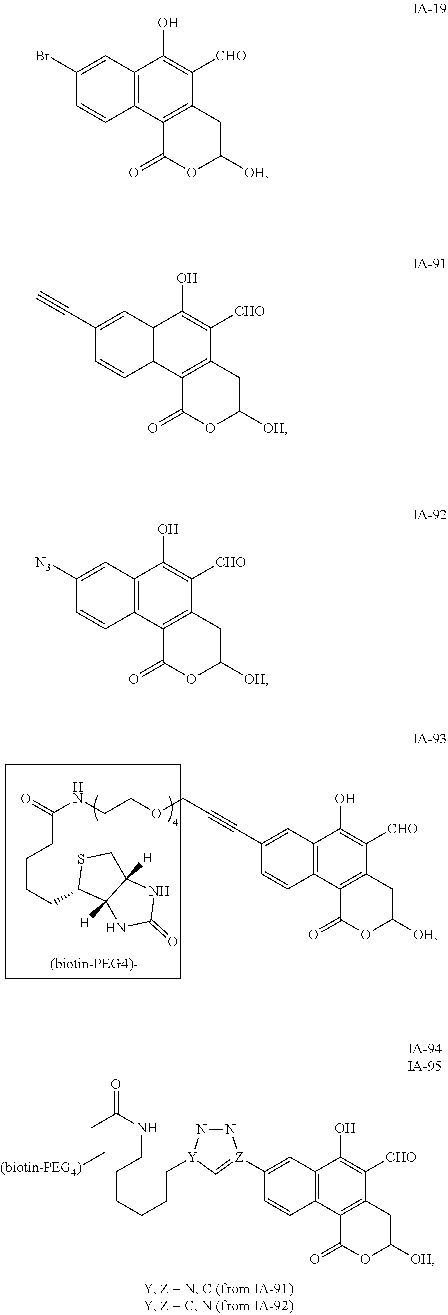

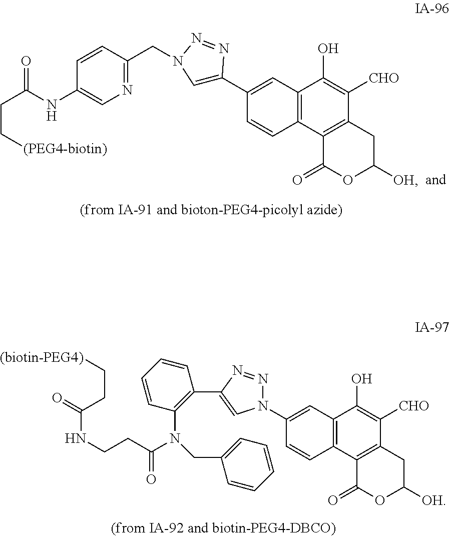

Another embodiment of this invention provides a compound that is one selected from the group of compounds consisting of one of the following structures:

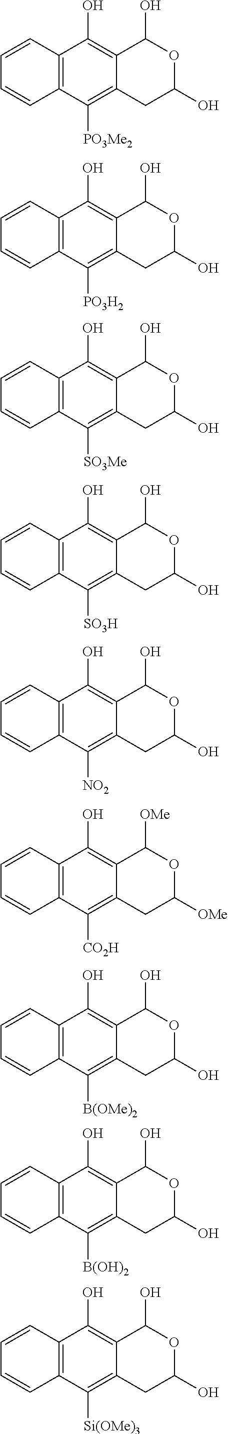

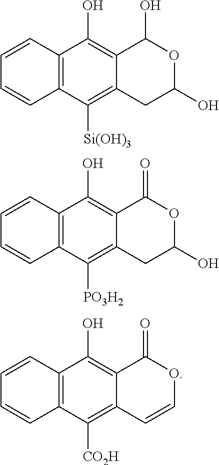

### BRIEF DESCRIPTION OF THE DRAWINGS

A full understanding of the invention may be gained from the following description of the embodiments of this invention when read in conjunction with the accompanying figures:

FIG. 1 shows several examples of the structures of the illudalic acid analogues of this invention, and more specifically shows convergent benzannulation (unoptimized) for the synthesis of illudalic acid and illudalog compounds of this invention.

FIG. 2 shows illudalic acid and key structural elements (top left of FIG. 2); simulated LAR docking from reference 17 (neopentylene ring fusion circled for clarity) (top right of FIG. 2), and the postulated illudalic acid ligation with a truncated bicyclic illudalic acid analogue of this invention, namely, 7-BIA (bottom of FIG. 2).

FIG. 3 shows the Woodward synthesis of illudalic acid (ref 16, 17 steps/1.1% overall), later updated by Shen (ref 17, 16 steps/2.4% overall) and then by the present invention (ref 15, 20 steps/7.7% overall) (see top of FIG. 3); Shen synthesis of 7-BIA (yields taken from ref 14; 13 steps/17% overall) (see bottom of FIG. 3).

FIG. 4 shows the convergent benzannulation approach of tis invention to tricyclic illudalic acid analogues, also referred to herein as “illudalogs” (top—FIG. 4), and alternative pharmacophore functionality of the trifunctional illudalog compounds of this invention for altering selectivity and efficacy.

FIG. 5 shows illudalic acid and illudalog compounds of this invention that have been prepared.

FIG. 6 shows the synthesis of illudalic acid (IA-1), following the Woodward/Shen approach (16 steps, 12.5% from 1; 20 steps, 7.7% from dimedone), producing >50 mg of illudalic acid by this approach (left—FIG. 6), and truncated bicyclic analogue IA-0 (11 steps) (right—FIG. 6).

FIG. 7 shows that Illudalic acid is more potent and its methyl ether is more selective for LAR; data from reference 15.

FIG. 8 shows convergent benzannulation (unoptimized) for the synthesis of naphthalene-(top—FIG. 8) and indane- (bottom left—FIG. 8) based illudalogs; Illudalic acid (IA-1) and its methyl ether (IA-2), and the 8 new illudalogs (IA-3 to IA-10) (bottom right—FIG. 8) of this invention.

FIG. 9 shows LAR-PTP inhibitory activity and IC₅₀ for 7-BIA, illudalic acid (IA-1), and new illudalogs at pH 6.5 (inset graph: at pH 8.0).

FIG. 10 shows the synthesis of 20 illudalogs pf this invention from 10 commercial benzoic acids: 10 phenols (IA-11-IA-29, odd numbers) and 10 methyl ethers (IA-12-IA-30 even numbers) (see top—FIG. 10); combinatorial synthesis of 25 illudalogs of this invention bearing diverse ethers: IA-31-IA-55 (see lower left inset box—FIG. 10).

FIG. 11 shows the synthesis of ethynyl and azido illudalogs (IA-91 and IA-92) and five examples of biotinylated probes (IA-93-IA-97). The corresponding phenol methyl ethers (IA-98-IA-104) will also be prepared but are not shown in FIG. 11. The structure of the (biotin-PEG4)—tag and linker is highlighted in the shadow box on IA-9. Biotin itself is depicted on the upper right of FIG. 11.

FIG. 12 shows the preliminary (unoptimized) synthesis of IA-3 and IA-4, reprised from FIG. 8 (top—FIG. 12), and the 5-step synthesis of illudalic acid from 6. Initial plan: X═Br; Z═N(OMe)Me (Weinreb amide); R=t-Bu; R′=Et (bottom—FIG. 12).

FIG. 13 shows alkylation of the phenol to provide diverse alkyl ethers (top—FIG. 13), and substitution of the phenol by metal-catalyzed cross-coupling reactions (bottom—FIG. 13).

FIG. 14 shows annulation of the pharmacophore onto diverse scaffolds, with examples including carbocycles, heterocycles, and bicycles (all compounds of this invention).

FIG. 15 shows phenol-directed reduction of Weinreb amide and acid-catalyzed hydrolysis to IA3 (top), a 3-step route for multigram-scale synthesis of IA3 (4-step LLS (middle), and 5-step LLS synthesis of IA2-8Me2 (bottom).

FIG. 16 shows screening of the illudalog compounds of this invention and 7-BIA against LAR-PTPs at pH 7.5 (in order from left to right for each respective illudalog compound set forth on the x-axis of FIG. 16. PTPR^{δ}, PTPRα, and LAR, respectively) FIG. 17 shows an exploratory functionalization of positions R-7-R 10 for compound of this invention IA3-R⁷⁻¹⁰.

FIG. 18 shows an alternative 1-step synthesis of IA3 by acid-catalyzed annulation of homophthalic anhydride and 4-pyrone.

FIG. 19 shows illudalogs compounds of this invention from various a-, bi-, and cyclic ketones and from Diels-Alder adducts of 3-bromopropiolates (BrC═CCO₂R).

FIG. 20 shows alternative pharmocophore functionality of illudalogs compounds of this invention for altering selectivity and efficacy (top—FIG. 20), and a synthesis route of Ian-1P series (bottom—FIG. 20).

FIG. 21 shows pharmacophore “knock-out” compounds as negative controls lacking individual functional groups.

FIG. 22 shows synthesis of fomajorin D and reduction to IA1-1Me (top), and synthesis of IA1-1Z variants by analogy to fomajorin D synthesis (bottom left), and alternative illudalog annulation from 4-pyrone (bottom right).

FIG. 23 shows synthesis of IA3-8Br and IA3-8C2H (top—FIG. 23), synthesis of IA3-8N3 and IA3-Bt1, and alternate azide IA3-879N3, and the structure of biotin (middle—FIG. 23), and azide-alkyne click coupling options for preparing IA3-Bt2 and IA3-Bt5 using CuAAC and SPAAC (bottom—FIG. 23).

### DESCRIPTION OF THE EMBODIMENTS OF THE INVENTION

This invention provides a general methodology for the design and synthesis of selective phosphatase inhibitors to support drug discovery efforts. This invention provides potent, selective, and cell-permeable LAR-PTP inhibitors based on illudalic acid. Previous work by us and others supports the central hypothesis that illudalic acid and analogues first bind reversibly in the PTP catalytic domain, then ligate covalently to a conserved active-site cysteine residue. However, the exact molecular binding interactions are unknown, and chemical synthesis has been a bottleneck as noted above. Preliminary results supporting this invention include new potent and selective tricyclic analogues that can be made in 4 linear steps. This invention provides for the synthesis of selective phosphatase inhibitors (i.e. the compounds of this invention) based on illudalic acid. The compounds of this invention provide new therapeutics targeting LAR-PTPs and guide the design of future selective PTP inhibitors.

In one embodiment of this invention, a method of synthesizing illudalic acid is provided comprising carrying out each of the five steps of the following scheme:

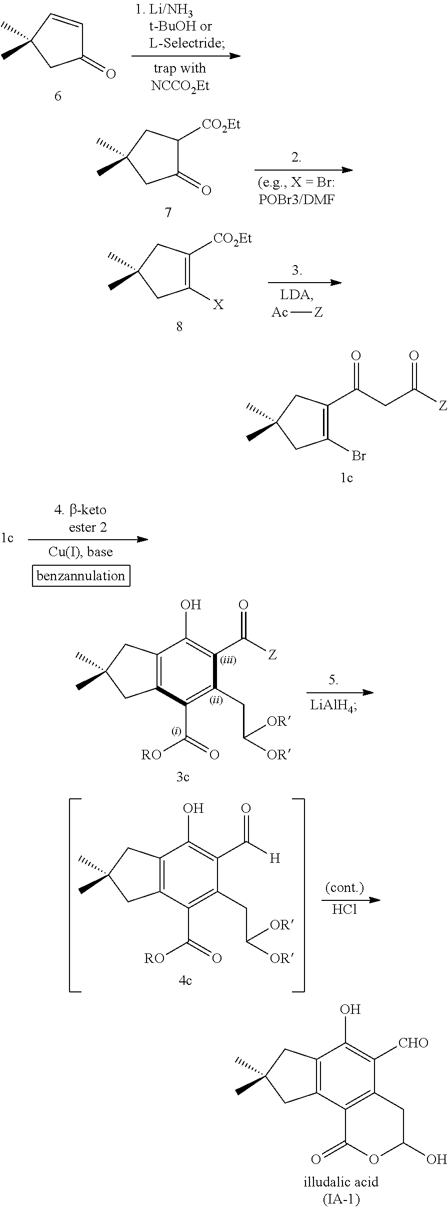

Another embodiment of this invention provides a general method of preparing illudalic acid comprising providing benzannulation of a β-keto amide having a formula 3a

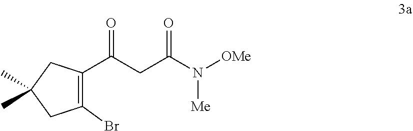

with a β-keto ester having a formula 4

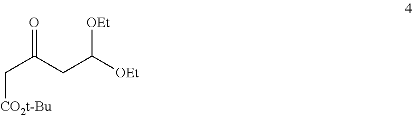 [SMILES: CCOC(CC(=O)CC(=O)OC(C)(C)C)OCC]

to produce a precursor compound having a formula 9a

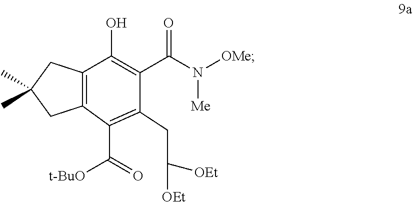

carrying out reduction of said precursor compound 9a with LiAlH₄ to form a reduced precursor compound having a formula 12

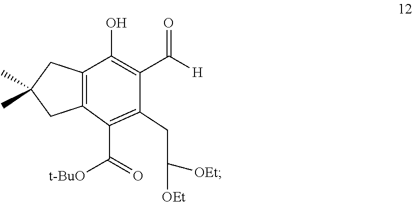

carrying out acid hydrolysis of said reduced precursor compound 12 to form illudalic acid having a formula IA1

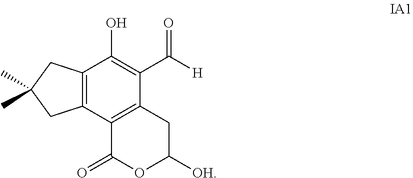

In another embodiment of this invention, this method includes subjecting a tetralin compound analogous to said reduced precursor compound 12 to two equivalents of LiAlH₄ to form a tetralin compound having a formula IA2

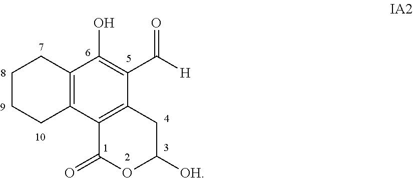 [SMILES: O=Cc1c(O)c2c(c3c1CC(O)OC3=O)CCCC2]

In another embodiment of this invention, this method includes subjecting a naphthalene compound analogous to said reduced precursor compound 12 to two equivalents of LiAlH₄ to form a naphthalene compound having a formula IA3

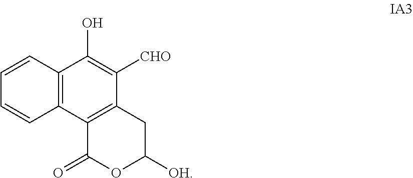 [SMILES: O=Cc1c2c(c3ccccc3c1O)C(=O)OC(O)C2]

In another embodiment of this invention, a compound is provided having a formula 13:

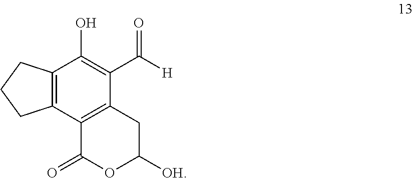 [SMILES: O=Cc1c(O)c2c(c3c1CC(O)OC3=O)CCC2]

In another embodiment of this invention, a compound is provided having a formula 14:

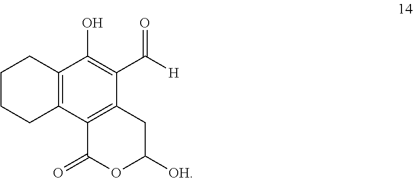 [SMILES: O=Cc1c(O)c2c(c3c1CC(O)OC3=O)CCCC2]

In another embodiment of this invention, a compound is provided having a formula 15:

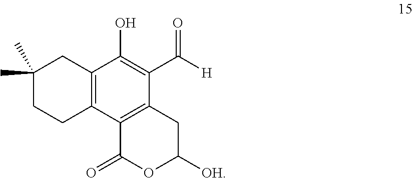

In another embodiment of this invention, a compound is provided having a formula 16:

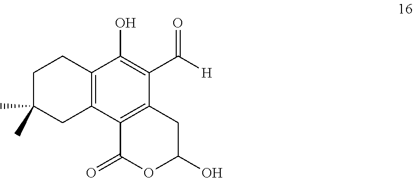

Another embodiment of this invention provides a compound that is one selected from the group of compounds consisting of IA1-6OMe, IA1-8H2, and IA1-6OMe-8H2, having one of a formula:

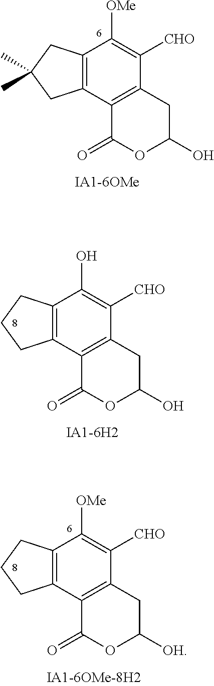

Another embodiment of this invention provides a compound that is one selected from the group of compounds consisting of IA2, IA2-8Me2, and IA2-9Me2, having one of a formula:

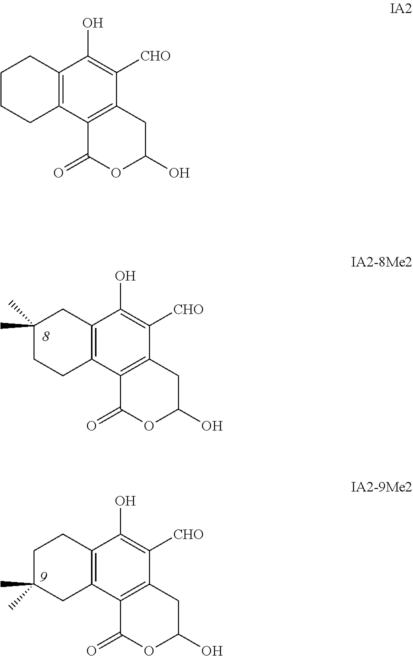

Another embodiment of this invention provides a compound that is one selected from the group of compounds consisting of IA3, IA3-6OMe, IA3-6OEt, IA3-6OC3H3, IA3-6OBn, IA3-7Me, IA3-8Me, IA3-9Me, IA3-8Br, IA3-8C2H, IA3-8C1, IA3-9Cl, IA3-9tBu, IA3-9CF3, IA3-7OMe, IA3-8OMe, IA3-89OMe, IA3-89F, and IA3-6OMe89F, having one of a formula:

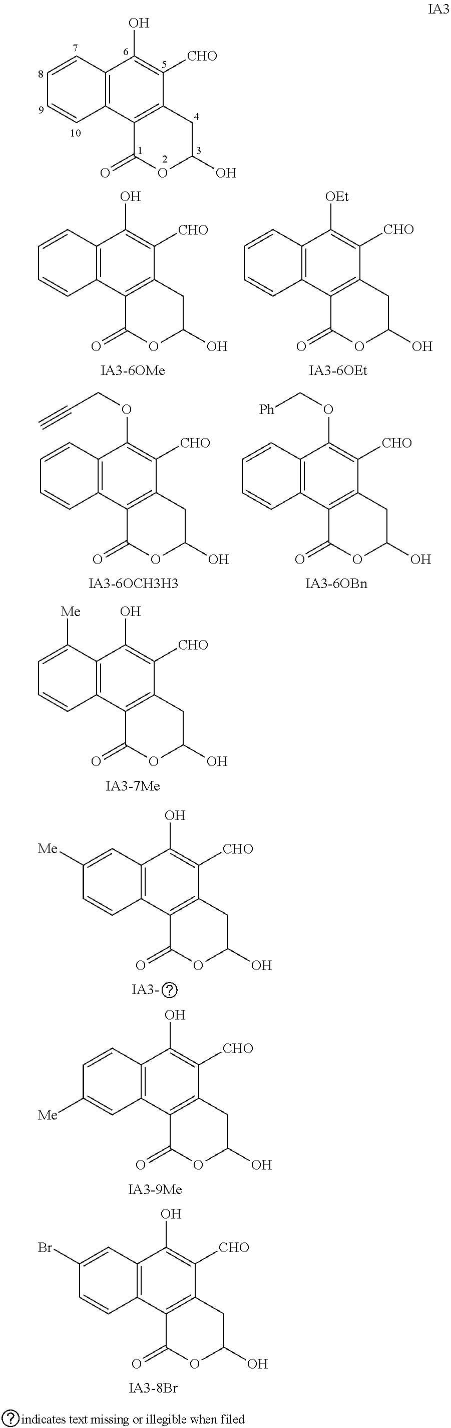

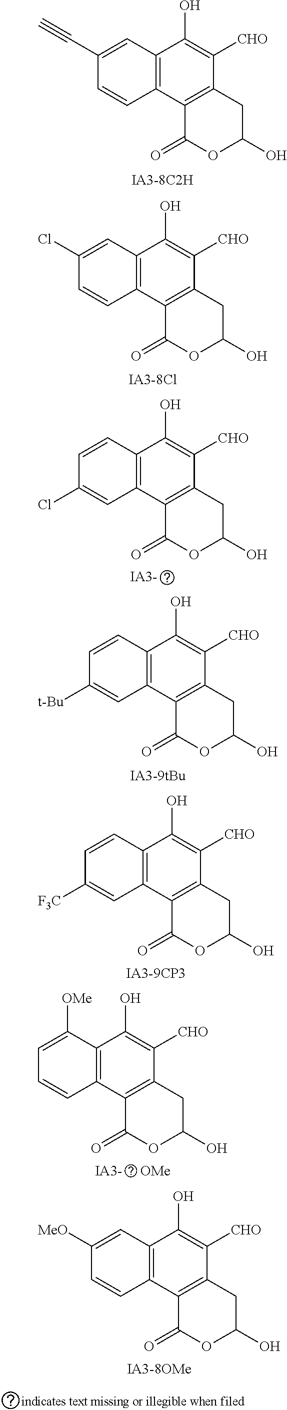

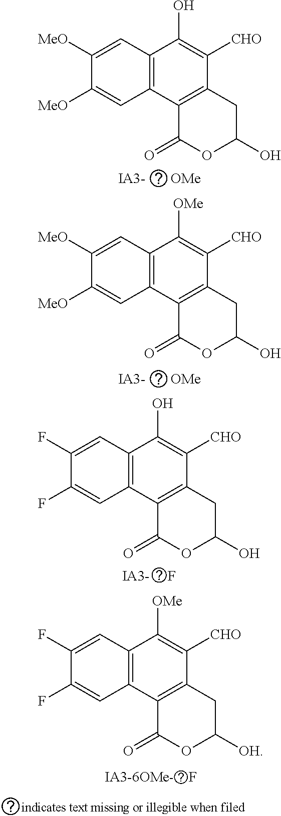 [SMILES: COc1cc2c(O)c(C=O)c3c(c2cc1OC)C(=O)OC(O)C3 ; COc1cc2c(OC)c(C=O)c3c(c2cc1OC)C(=O)OC(O)C3 ; O=Cc1c2c(c3cc(F)c(F)cc3c1O)C(=O)OC(O)C2 ; COc1c(C=O)c2c(c3cc(F)c(F)cc13)C(=O)OC(O)C2]

Another embodiment of this invention provides a compound that is one selected from the group of compounds consisting of: IA-5, IA-7, IA-8, IA-9, and IA-10, having one of a formula:

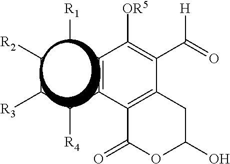

IA-5: R₁, R₂, R₃, and R₄═H, and R⁵═H
IA-6: R₁, R₂, R₃, and R₄═H, and R⁵=Me (i.e. CH₃)
IA-7: R₁ and R₄═H, and R₂ and R₃=MeO, and R⁵═H
IA-8: R₁, R₃, R₄, and R₂═H, and R⁵═H
IA-9: R₁, R₂, and R₄═H, and R₃═Cl, and R⁵═H
IA-10: R₁, R₂, and R₄═H, and R₃=t-Bu, and R⁵═H.

Another embodiment of this invention provides a compound of a formula IA-4:

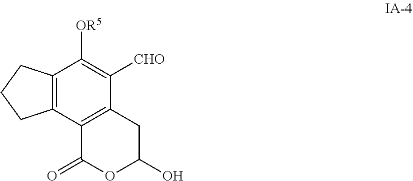

wherein R⁵ is a methyl group.

Another embodiment of this invention provides a compound that is one selected from the group of compounds consisting of IA-11, IA-12, IA-13, IA-14, IA-15, IA-16, IA-17, IA-18, IA-19, IA-20, IA-21, IA-22, IA-23, IA-24, IA-25, IA-26, IA-27, IA-28, IA-29, and IA-30, having one of a formula:

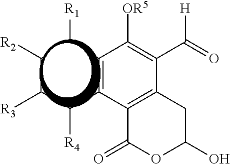

IA-11: R₁=Me, R⁵═H, and R₂-R₄═H
IA-12: R₁=Me, R⁵=Me, and R₂—R₄═H
IA-13: R₂=Me, R⁵═H, and R₁, R₃, and R₄═H
IA-14 R₂=Me, R⁵=Me, and R₁, R₃, and R₄ ═H
IA-15: R₃=Me, R⁵═H, and R₁, R₂, and R₄═H
IA-16: R₃=Me, R⁵=Me, and R₁, R₂, and R₄═H
IA-17: R₄=Me, R⁵═H, and R₁—R₃═H
IA-18: R₄=Me, R⁵=Me, and R₁—R₃═H
IA-19: R₂═Br, R⁵═H, and R₁, R₃, and R₄═H
IA-20: R₂═Br, R⁵=Me, and R₁, R₃, and R₄═H
IA-21: R₁═OMe, R⁵═H, and R₂—R₄═H
IA-22: R₁═OMe, R⁵=Me, and R₂—R₄═H
IA-23: R₂═OMe, R⁵═H, and R₁, R₃, and R₄═H
IA-24: R₂═OMe, R⁵=Me, and R₁, R₃, and R₄═H
IA-25: R₃═OMe, R⁵═H, and R₁, R₂, and R₄═H
IA-26: R₃═OMe, R⁵=Me, and R₁, R₂, and R₄═H
IA-27: R₄═OMe, R⁵═H, and R₁—R₃═H
IA-28: R₄═OMe, R⁵=Me, and R₁—R₃═H
IA-29: R₁-R₃═F, R⁵═H, and R₄═H
IA-30: R₁—R₃═F, R⁵=Me, and R₄═H.

Another embodiment of this invention provides a compound that is one selected from the group of compounds consisting of IA-31-IA-55, having one of a formula:

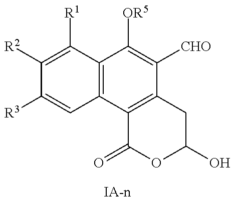

| | R¹⁻³ = H | R^{2,3} = OMe | R¹⁻³ = F | R¹ = Me | R² = Br |
|---|---|---|---|---|---|
| R⁵ = ethyl | IA-31 | IA-36 | IA-41 | IA-46 | IA-51 |
| R⁵ = butyl | IA-32 | IA-37 | IA-42 | IA-47 | IA-52 |
| R⁵ = benzyl | IA-33 | IA-38 | IA-43 | IA-48 | IA-53 |
| R⁵ = propargyl | IA-34 | IA-39 | IA-44 | IA-49 | IA-54 |
| R⁵ = CH₂CO₂Et | IA-35 | IA-40 | IA-45 | IA-50 | IA-55 |

wherein IA-36-IA-40: R₁═H, wherein IA-46-IA-50: R₂ and R₃═H; and wherein IA-51-IA 55: R₁ and R₃═H.

Another embodiment of this invention provides a compound that is one selected from the group of compounds consisting of IA-105-IA108, having one of a formula:

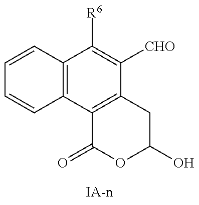

| | |
|---|---|
| IA-105: | R⁶ = methyl |
| IA-106: | R⁶ = ethynyl |
| IA-107: | R⁶ = allyl |
| IA-108: | R⁶ = phenyl |
| IA-109: | R⁶ = 2-furyl |
| IA-110: | R⁶ = H |

Another embodiment of this invention provides a compound that is one selected from the group of compounds consisting of IA3-5Me, IA3-5OMe, IA3-3H, and IA3-1Me, having one of a formula:

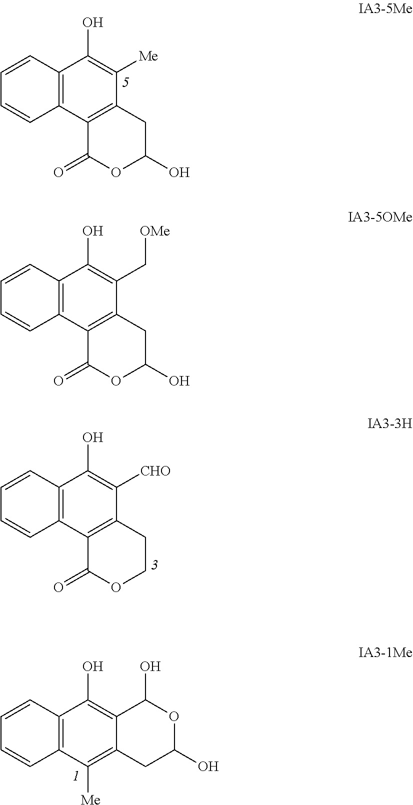 [SMILES: Cc1c2c(c3ccccc3c1O)C(=O)OC(O)C2 ; COCc1c2c(c3ccccc3c1O)C(=O)OC(O)C2 ; O=Cc1c2c(c3ccccc3c1O)C(=O)OCC2 ; Cc1c2c(c(O)c3ccccc13)C(O)OC(O)C2]

Another embodiment of this invention provides a compound that is one selected from the group of compounds consisting of IA-19, and IA-91-IA-97, having one of a formula:

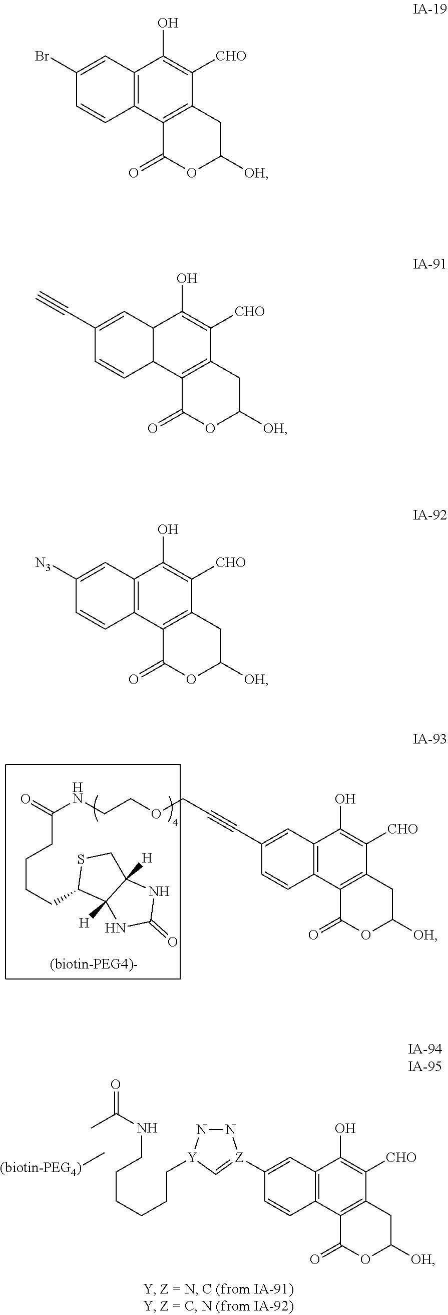

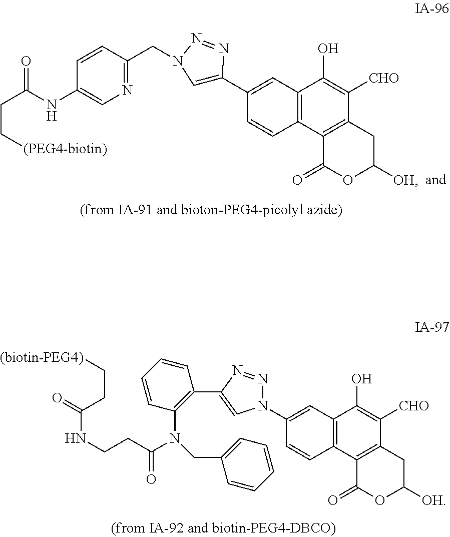

Another embodiment of this invention provides a compound that is one selected from the group of compounds consisting of one of the following structures:

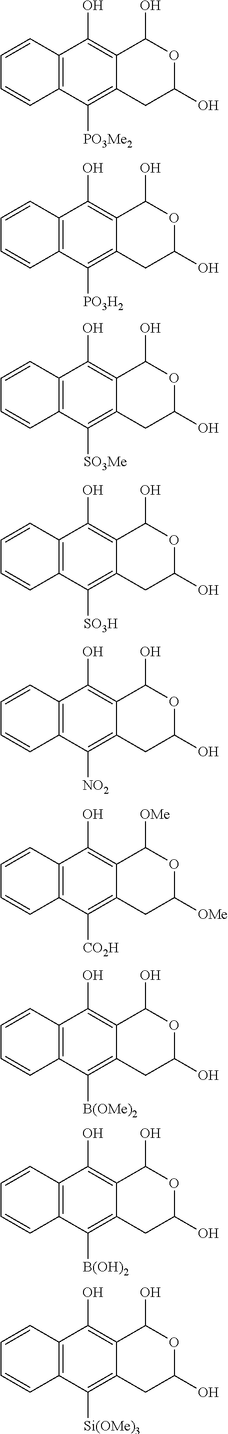

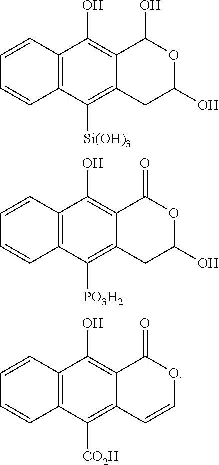

### Selective LAR-PTP Inhibitors Based on Illudalic Acid

We make and evaluate illudalic acid analogues in vitro. We assess their inhibitory activity against a panel of PTPs; map their relevant binding interactions with LAR, PTPR^{δ}, and PTPR^{δ} using thermodynamic and kinetic binding data,¹⁵ X-ray crystallography,²⁸ and computational modeling; and create a detailed SAR profile for their inhibition of LAR-PTPs. This invention provides that as covalent ligation occurs to the conserved active-site cysteine residue, the bulk of the inhibitor scaffold projects into non-conserved regions of the catalytic domain active sites. Understanding the unique molecular recognition elements for each LAR-PTP subfamily member will reveal design parameters for potent and selective LAR-PTP inhibitors, which will be further tested and validated in subsequent iterations of synthetic illudalic acid analogues. Preliminary data suggest that our tricyclic inhibitor scaffolds with optional phenol methylation provide potency and selectivity for LAR-PTPs over other PTPs, and can even promote selectivity within the LAR-PTP subfamily.

### Identification and Characterization of Intracellular Activity of Illudalic Acid-Based LAR-PTP Inhibitors

We evaluate functional activity and off-target interactions in vivo (cells), examining bioavailability, compatibility, and cellular efficacy (potency and selectivity). Aim 2 is that illudalic acid analogues will be cell-permeable, selective, and effective at intracellular inhibition of LAR-PTP activity. We will use alkyne- and azide-modified analogues and click chemistry to attach biotin tags in live cells and cell lysates (for pull-down assays) after covalent ligation²⁹ of the illudalic acid analogues to their intracellular targets.

### Versatile and Scalable Syntheses of Illudalic Acid and Analogues

We provide a 5-step synthesis of illudalic acid from 4,4-dimethylcyclopentenone, based on naphthalene benzannulation methodology³⁰ and provide results from 4-step syntheses of tricyclic illudalic acid analogues. Previous syntheses, in contrast, require 16 linear steps to illudalic acid, and 11 linear steps to truncated bicyclic analogues.¹⁴⁸ Key indane and naphthalene benzannulation reactions are optimized to support versatile, gram-scale syntheses of the natural product and diverse analogues for future animal studies.

We provide the structures of potent, selective, and cell-permeable LAR-PTP inhibitors and chemical probes for drug discovery efforts. We develop detailed SAR profiles for illudalic acid-based PTP inhibitors and atomic-level understanding of molecular recognition and covalent ligation mechanisms.

Therapeutic Potential of Selective LAR-PTP Inhibitors of this Invention

This invention focuses on the LAR subfamily of receptor-type PTPs, referred to herein as LAR-PTPs. The LAR-PTPs (LAR, PTPR^{δ}, and PTPR^{δ}) are transmembrane, multi-domain proteins consisting of extracellular immunoglobulin-like and fibronectin-like domains, a hydrophobic transmembrane region, and two cytosolic PTP domains; only one of these (the membrane-proximal D1 domain) is believed to have significant catalytic activity.⁵ The LAR-PTPs are implicated in axon guidance and neuronal growth^{2a,5} as well as the regulation of cell-cell interactions.⁵ The LAR-PTPs are potential therapeutic targets for cancer, metabolic disorders, nerve regeneration, and stimulant addiction.^{5,14} However, many questions remain about their biological roles in each of these pathways and their viability as therapeutic targets. To this end, the systematic development, optimization, and validation of potent and selective inhibitors of each of the individual LAR-PTPs is instrumental in interrogating the biological activity of these enzymes, validating each as a therapeutic target, and providing lead compounds. Illudalic acid and its unique trifunctional pharmacophores of this invention provide insights into how to answer these important biological questions.

### Background Synthesis and Molecular Pharmacology of Illudalic Acid

Illudalic acid (IA-1, FIG. 2) was first identified in 1952 as an “inactive acidic compound” from the toxic jack o'lantern mushroom (Clitocybe illudens).³¹ It was structurally characterized in 1969,¹³ synthesized in 1977,¹⁶ and emerged from a HTS campaign as the “first potent, selective” LAR phosphatase inhibitor in 2005.¹² Subsequent efforts identified the postulated trifunctional pharmacophore,¹⁷ provided insights into its mechanism of action,^{15,17} and revealed potential for in vivo efficacy.¹⁴ The IA-1 structure of illudalic acid was recently used as a positive control in virtual screening and design of LAR-PTP inhibitors, although these in silico simulations did not account for the two-stage mechanism of action (and role of the free dialdehyde, next paragraph) in the binding event.³² The illudalic acid pharmacophore, masked as an ortho-formyl hydroxy-lactone, is critical for LAR-PTP inhibition. The postulated mechanism of action is that illudalic acid binds noncovalently first in the LAR catalytic domain active site, then ligates covalently via its free dialdehyde to the conserved active-site cysteine residue (Cys1522). A molecular docking simulation showing illudalic acid covalently bound to Cys1522 in the LAR catalytic domain active site (FIG. 2, top right) places the unmasked carboxylate near the conserved arginine residue (Arg1528), perhaps forming a salt bridge. The bulk of the tricyclic scaffold projects into an open region of the active site.

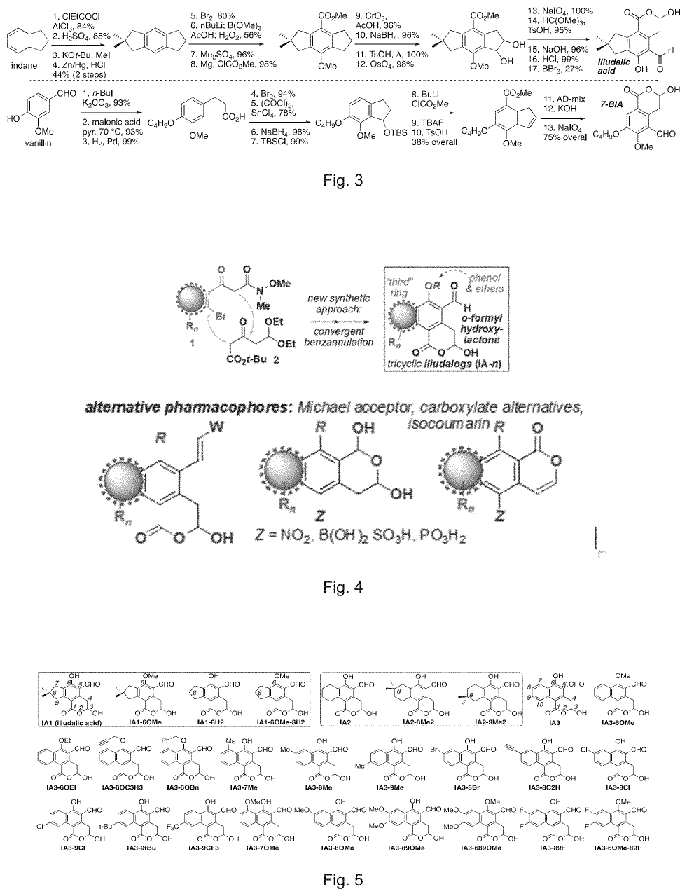

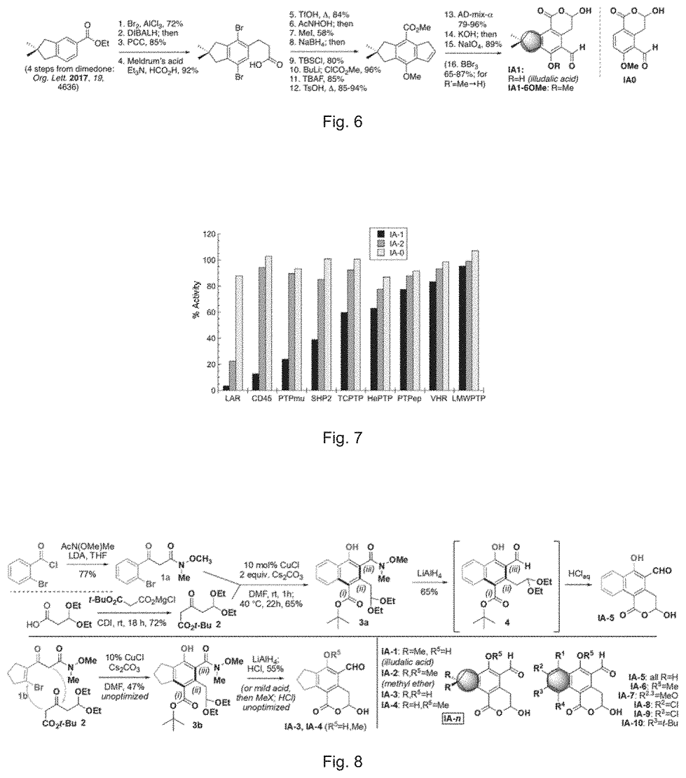

The tricyclic scaffold and phenol are important for potency and selectivity. We reported that illudalic acid inhibits LAR with measured IC₅₀ 2.1+0.2 μM, whereas its phenol methyl ether is less potent (IC₅₀ 55±6 μM) but more selective for LAR over the other PTPs that we examined¹⁵ (see Preliminary Results). A bicyclic analogue resulting from simple deletion of the neopentylene ring fusion (IA-0, cf. FIG. 6) was inactive in our standard assays. Shen and co-workers reported that LAR inhibitory activity can be observed in this (IA-0) and other truncated bicyclic analogues, as well as in the natural product itself, by employing a modified two-stage assay: “compounds were pre-incubated with LAR enzyme at pH 8 and the enzyme activity was assayed at pH 6. Otherwise, none of these compounds show any LAR inhibitory activity if they are incubated and tested at pH 6.”¹⁷ Although the physiological relevance of this pH switch is dubious, the two-stage variable-pH assay aligns with the postulated two-stage binding mechanism, and it likely facilitated follow-up studies involving 7-alkoxy bicyclic analogues.¹⁸ The 7-alkoxy substituents on these bicyclic analogues occupy similar space as the deleted neopentylene ring fusion and were (marginally) easier to prepare than illudalic acid (13 steps, cf. FIG. 3). One of these, the 7-(n-butoxy) analogue known as 7-BIA (FIG. 2, bottom right), was found to inhibit the LAR-PTPs (LAR, PTPR^{δ}, and PTPR^{δ}) selectively over other PTPs and to reduce the cocaine reward in mice (linked to PTPR,^{δ} inhibition).¹⁴ These observations establish in vivo efficacy of the illudalic acid pharmacophore. Note that these LAR-PTP activities and selectivities were established using the two-stage variable-pH assay described above, but our preliminary assays under standard conditions show similar trends (vide infra).

Advances in chemical synthesis are critical for realizing the chemotherapeutic potential of illudalic acid and related LAR-PTP inhibitors. Although the full tricyclic scaffold of illudalic acid is important for potency, the first synthetic analogues were truncated bicycles designed for synthetic expediency. The current synthetic methods were pioneered by Woodward and Hoye in 1977 with their 17-step synthesis of illudalic acid (1.1% overall yield), albeit with unresolved regiocontrol and other concerns (FIG. 3, top).¹⁶ Once its pharmaceutical potential was recognized, Shen updated the Woodward synthesis to produce illudalic acid in 16 steps and 2.4% overall yield,¹⁷ and developed a 13-step route to bicyclic 7-alkoxy analogues,¹⁸ albeit with the variable alkoxy group introduced in step 1. Uhl later used this route to produce 7-BIA in 17% overall yield (FIG. 3, bottom). We merged these approaches with on-going methodology³³ to produce illudalic acid in 20 steps (7.7% yield) from dimedone.¹⁵ The established synthetic approach to the illudalic acid pharmacophore is not viable, or is at least a liability, for rapid and/or large-scale synthesis of diverse analogues for discovery and development of selective PTP inhibitors.

As is discussed herein, we have made significant advances in the synthesis of the illudalic acid pharmacophore that position us to provide potent, selective, and bioavailable inhibitors of the LAR-PTP family of enzymes for the first time based on rapid synthesis of diverse tricyclic analogues. We will optimize and validate (in vitro and in vivo) our selective inhibitors of LAR, PTPR^{δ}, and PTPR^{δ} as potential therapeutic lead compounds.

The therapeutic potential of the illudalic acid pharmacophore, and specifically 7-BIA (set forth herein) and innovations in the synthesis of illudalic acid provided by this invention, create phosphatase inhibitor compounds based on illudalic acid. In mice, heterozygous Ptprd knockouts show reduced cocaine conditioned place preference and self-administration compared to wild-type. 7-BIA, a truncated analog of illudalic acid, reduces cocaine conditioned place preference and self-administration in wild-type mice, but not in the Ptprd knockout mice. No evidence of toxicity was observed in mice dosed with 7-BIA up to ˜60 mg/kg (the solubility limit). These observations establish in vivo efficacy of the illudalic acid pharmacophore. In vitro, 7-BIA inhibits PTPRD with an estimated IC₅₀ of 1-3 mM. Noted limitations of 7-BIA include its solubility, stability, and lack of broader in vivo pharmacological and toxicological analyses.

Methods and Compounds of this Invention

The major innovations driving this research are: (1) a synthetic approach that enables illudalic acid and novel analogues (herein referred to as “illudalogs”) to be prepared in as few as 4-5 steps (compared to known current syntheses that require 11-20 steps), and (2) the application of illudalog compounds of this invention as potent and selective PTP inhibitors for elucidating the biology and therapeutic potential of these important but understudied enzymes. We establish clear structure-activity relationship (SAR) profiles, develop selective phosphatase inhibitors, and guide future drug discovery and development of novel PTP-targeting chemotherapeutics.

The convergent benzannulation approach is strategically and tactically superior to current (linear) syntheses This invention's synthetic approach involves condensing two components of similar size and complexity to assemble the arene core, followed by coordinated functional group interconversion to establish the pharmacophore (FIG. 4). One component, β-keto ester 2, will be a common building block for illudalic acid and all proposed illudalogs. The other, β-keto amide 1, will variably incorporate the structural diversity. These two components, one constant and one variable, encode the trifunctional pharmacophore, phenol, and “third ring” of the proposed tricyclic illudalogs. The plan to assemble them in one key benzannulation step is strategically innovative in the context of illudalic acid and in the design and synthesis of selective phosphatase inhibitors, and broadly attractive from a general chemical synthesis perspective.³⁴ The synthetic approach is also tactically innovative for its deliberate selection of functional group forerunners to the target ortho-formyl hydroxy-lactone, exemplified here as a tert-butyl ester, diethyl acetal, and Weinreb amide. The unique properties of this densely functionalized system allow for (phenol-directed) reduction of a Weinreb amide in preference to an ester, with a strong aqueous acid quench then providing target synthetic illudalogs in only one post-benzannulation operation. β-Keto ester 2 is available in 2 linear steps on multigram-scale, and most β-keto amide components are available in 1 step from 2-bromobenzoic acid derivatives, thus providing diverse tricyclic illudalogs in 4 steps (longest linear sequence, LLS).
The illudalog compounds of this invention provide the first selective LAR-PTP inhibitors and chemical probes Developing inhibitors with selectivity for one PTP family of enzymes over others is challenging, and selective inhibition within the LAR-PTP subfamily is an unsolved problem. Previous analogues of illudalic acid are truncated bicyclic structures designed for ease of synthesis rather than pharmacological efficacy. Current understanding of illudalic acid/LAR binding is that the trifunctional pharmacophore binds to the conserved P-loop—covalently to Cys1522 and non-covalently to Arg1528—and projects into a non-conserved void in the LAR catalytic domain active site. We have images showing illudalic acid is bound at the P-loop (Cys1522 and projecting into a void in the LAR catalytic domain active site.¹⁷ The greater potency of tricyclic structures¹⁵ and Shen's use of two-stage variable-pH assays for bicyclic analogues¹⁷ are consistent with this binding model. Based on this model, we provide tricyclic analogues designed to (1) occupy this void with rigid and variable elements of chemical structure for enhanced potency and selectivity, and (2) exploit this void as a point of attachment for chemical probes, which will provide important data on cellular activity and any off-target interactions. Our designs also focus on diversification at the phenol, based on preliminary data and observations that phenol methylation enhances potency and selectivity within the LAR-PTP family in a pH-dependent manner (vide infra). Our data and observations retrospectively rationalize Uhl's identification of 7-B1A (for which the phenol is blocked as a methyl ether) as a PTPR^{δ} inhibitor, and prospectively suggest opportunities to tune inhibitor selectivity within the LAR-PTP subfamily.

These innovations in molecular design and synthesis support the proposed multidisciplinary approach to developing potent, selective, and cell-permeable LAR-PTP inhibitors based on illudalic acid. The approach itself is innovative in targeting the conserved cysteine residue in PTP catalytic domain active sites with the trifunctional pharmacophore from illudalic acid, while strategically varying its rigid tricyclic scaffold and substitution patterns to probe and exploit variations in the catalytic domains of different PTPs. By aligning the conserved and variable structural features in our inhibitors with the conserved and variable features of the PTP catalytic domains, we create innovative design blueprints for PTP-targeted chemotherapeutics. Ultimately, the application of inhibitors developed here will provide novel insights into the biology of LAR-PTPs and innovative approaches to the treatment of diseases.

Illudalic Acid Analogues (Compounds) of this Invention

This invention provides a general methodology for the design and synthesis of selective phosphatase inhibitors based on the unique trifunctional pharmacophore of illudalic acid. The focus here is on the identification, characterization, and development of potent, selective, and cell-permeable LAR-PTP inhibitors.

We make at least 110 novel analogues of illudalic acid (“illudalogs”) across at least three iterative design cycles, determine inhibitory activity against a panel of representative PTPs including the three LAR-PTPs, and map relevant binding interactions using a combination of thermodynamic and kinetic binding assay data, X-ray crystallography, and computational modeling, resulting in a detailed SAR profile for illudalic acid-based PTP inhibitors. Cell studies are expected to establish that illudalogs are cell permeable, and to provide important information on cellular activity and off-target binding interactions. The primary approach will be to make use of probe precursors bearing alkyne or azide handles for minimum structural perturbations, with intracellular click assembly of functional probes. Alternatively, the proposed biotin-tagged illudalogs could be prepared in vitro and delivered into the cell. Meanwhile, chemical synthesis has been a major bottleneck to the development of illudalic acid pharmacology. Our convergent benzannulation approach to synthesis of novel illudalog compounds provides a synthetic methodology, a 4-5-step (LLS) synthesis of illudalic acid.

The synthesis of illudalane sesquiterpenes in connection with methodology for making alkynes is known.³⁵ A 6-step synthesis of alcyopterosin A³⁶ and 7-8-step syntheses of illudinine³⁷ an alkaloid congener of illudalic acid has been developed. PTP-targeting chemical probes and inhibitors to advance the understanding of PTP enzymology and cell biology are known.³⁸

In our initial synthesis and pharmacology of illudalic acid, we leveraged indane 1 from prior methodology³³ as the starting material for a 16-step synthesis of illudalic acid (IA-1, FIG. 6; 12% yield from 1; 20 steps and 7.7% overall from dimedone).¹⁵ This synthetic route follows the established paths of Woodward¹⁶ and Shen,¹⁷ and it gave us timely access to milligram quantities of the natural product and simple analogues for preliminary pharmacological assessment.

We also prepared IA-0¹⁷ and 7-BIA¹⁸ by the established routes. First, illudalic acid (but not simplified bicyclic analogue IA-0) is active in standard phosphatase assays. Second, illudalic acid dose- and time-dependently inhibits LAR with an IC₅₀=2.1±0.2 μM as measured in standard assays, an initial thermodynamic binding affinity K₁=130±50 μM, and a kinetic covalent ligation rate k_{inact}=1.3±0.4 min-. The k_{inact}/K₁ ratio of 10⁴ corresponds to a protein deactivation half-life of t^{∞}_{1/2}=0.5 min under pseudo-first order conditions (infinite inhibitor concentration).³⁹ Third, methyl ether IA1-6OMe is less potent (IC₅₀=55±6 μM) but more selective for LAR compared to other PTPs, hinting at the significance of this methylation on selectivity (FIG. 6). This enhanced selectivity seems to emerge from a confluence of weak initial binding (Kᵢ) and fast subsequent ligation (k_{inact}): The Kᵢ, and k_{inact} for IA1-6OMe were too weak and too fast, respectively, to measure easily. Truncated analog IA0 (benzene vs. dimethylindane core) shows negligible inhibitory activity in these assays. Illudalic acid is more potent than its methyl ether is more selective for LAR (assay conditions 100 μM inhibitor, pH 6.5, at 22 degrees Centigrade.

Three key measurables and two structure-activity relationships (SAR) emerged from initial data. Inhibition depends on pH, Kᵢ, and k_{inact} which are measurable. Initial SAR suggest that truncated analogs are less active, and the phenol methyl ether may be more selective. Data from IA1-6OMe suggest that Kᵢ, and k_{inact} can be tuned independently, including by substitution at the 6-position. The effects of assay pH are presumably associated with inhibitor pKₐ, which we can tune with appropriate molecular designs. Although Shen showed that activity lost in truncated analogs can be recovered with acyclic substituents (cf. 7-BIA¹²), we prioritize rigid polycyclic core frameworks coupled with innovations in synthesis as a more robust approach.

The synthesis and pharmacological characterization of novel tricyclic illudalogs is provided herein. As discussed above, the synthetic approach focuses on convergent benzannulation, followed by one-pot conversion to the ortho-formyl hydroxy-lactone pharmacophore (FIG. 8). β-Keto amides 1a and 1b were prepared by Claisen-type condensation of the Weinreb acetamide with acid chlorides; the variable carboxylic acid derivative here is the primary source of diversity in the illudalogs produced thus far (IA-3-IA-10). $-Keto ester 2 is the constant annulation partner to deliver functional precursors to the conserved hydroxy-lactone moiety. We have prepared β-keto ester 2⁴⁰ on a multigram scale using a CDI-mediated decarboxylative coupling.⁴¹ The benzannulation is adapted from a known naphthol annulation.³⁰ Preliminary experiments confirm that the approach is viable for naphthalene- and indane-based illudalogs. The final one-pot reduction/hydrolysis is exemplified here for producing illudalog IA-5: treatment of naphthol 3a with LiAlH₄ and then strong aqueous acid produces IA-5, which precipitates from the mixture as a crystalline solid. Presumably, the deprotonated phenol directs reduction of the ortho-amide in preference to the para-ester. Milder workup releases o-hydroxy aldehydes 4 with the 1-butyl ester and diethyl acetal still intact, which is convenient for alkylation of the phenol (e.g., to IA-4 and IA-6).

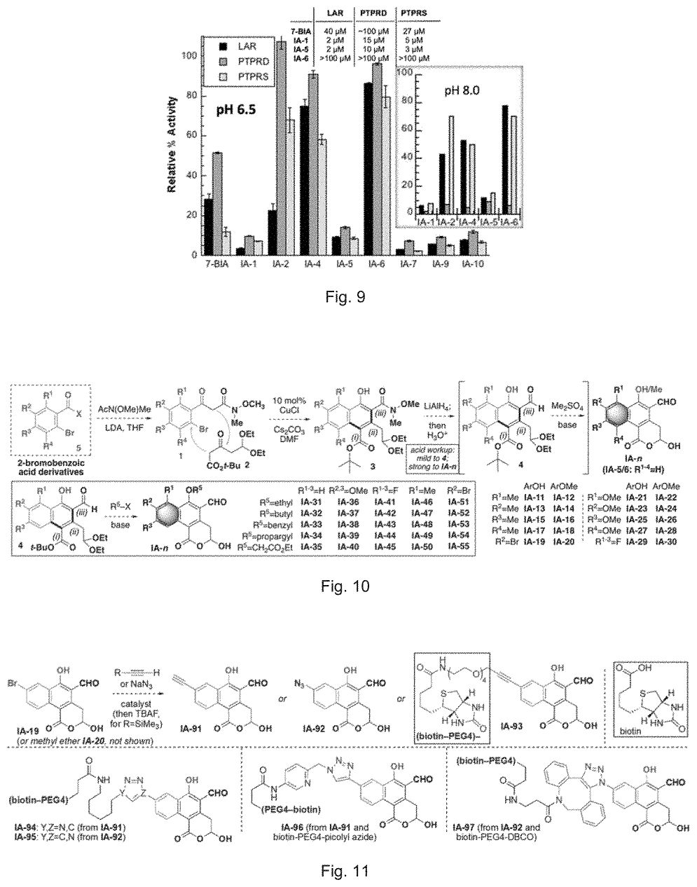

The illudalogs of this invention show inhibitory activity against LAR-PTPs in preliminary assays at pH 6.5 and pH 8 (FIG. 9). In all assays, 100 μM of compound was incubated with enzyme for 30 min prior to the assay. The free phenol and methyl ether illudalogs that we screened in both the naphthalene and indane series inhibit LAR-PTP activity at levels comparable to illudalic acid (IA-1) and its methyl ether (JA-2). Interestingly, the methyl ethers (IA-2, IA-4, and IA-6) appear to be potent and selective for PTPR^{δ} over LAR and PTPR^{δ} at pH 8 compared with the free phenols (FIG. 9, inset graph; note PTPR^{δ} inhibitory activity), but this potential PTPR^{δ} selectivity is not observed in the pH 6.5 assays. This apparent pH-dependence is consistent with the postulated two-stage binding mechanism and will be investigated further. There is some variation in potency and selectivity across the analogues, although these preliminary assays are not precise enough to support quantitative conclusions. Interestingly, 7-BIA, which has been used as a PTPR^{β} inhibitor in vivo,¹⁴ inhibits LAR and PTPR¹ more potently than PTPR^{δ} at pH 6.5. In addition, it is clear is that these novel tricyclic illudalogs are LAR-PTP inhibitors comparable to illudalic acid, and that substitution on the core structure (leaving the pharmacophore intact) provides opportunity to tune potency and selectivity for the LAR-PTPs.

### Present Compounds are Selective LAR-PTP Inhibitors Based on Illudalic Acid

While not desiring to be bound by any particular theory, we understand the binding parameters associated with LAR-PTP inhibition, with the central hypothesis being that illudalic acid and analogues first bind reversibly in the PTP catalytic domain, then ligate covalently to a conserved P-loop cysteine residue. While not desirous of being bound by any one theory, we believe that initial reversible binding is guided by interactions with non-conserved (variable) regions of the PTP active site. Understanding and tuning these initial noncovalent interactions is one key to developing potent, selective PTP inhibitors. Other important factors include the kinetics of rearrangement and ligation. All of these factors are tunable through structural modifications of the inhibitor.

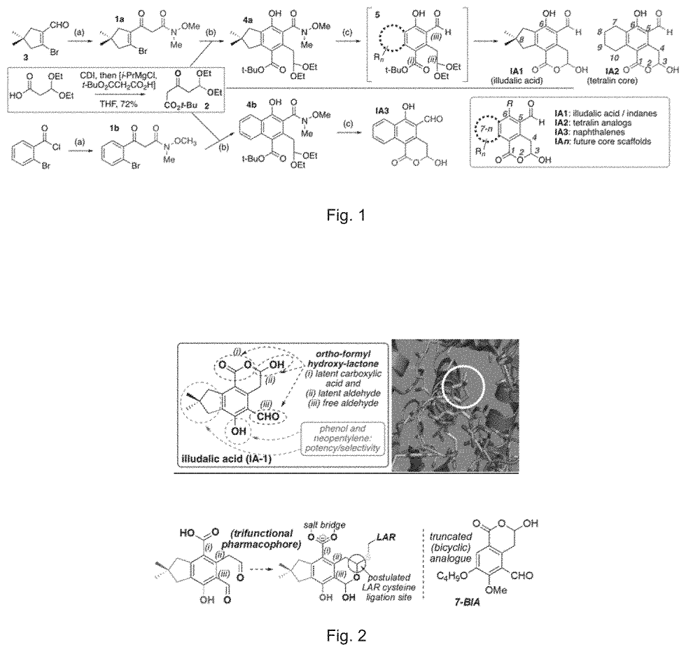

We provide herein more than 110 diverse tricyclic illudalogs of this invention, and establish their inhibitory activity against a panel of PTPs. All illudalogs and key synthetic intermediates (e.g. 4, which could suggest future prodrug opportunities) will be tested for inhibitory activity against the three LAR-PTPs and at least 9 other receptor- and nonreceptor-type PTPs, as done previously for illudalic acid (IA-1) and methyl ether IA-2 (cf. FIGS. 1, 7, and 9).¹⁵ Data from these inhibitory activity assays will identify preliminary SAR to guide the design and synthesis of subsequent illudalogs. Select illudalogs will undergo a more comprehensive battery of pharmacological assays, as we did for illudalic acid itself, to determine thermodynamic binding affinities (K₁), kinetic covalent ligation rates (k_{inact}), and t^{∞}_{1/2}, the hypothetical pseudo-first-order half-life of protein deactivation at infinite inhibitor concentration.³⁹ Meanwhile, co-crystallization of the LAR catalytic domain with illudalic acid and/or one of our initial illudalogs (e.g., IA-5) will allow for X-ray structural characterization of the inhibitor bound in the active site, providing static, three-dimensional images of the LAR catalytic domain with and without²⁸ bound illudalic acid, highlighting potential binding interactions. These molecular pharmacology and structural biology data will inform molecular dynamics simulations of the initial and terminal binding events. This process will then be repeated in an iterative cycle as the design parameters for selective LAR-PTP inhibitors become apparent.

Synthesis of Illudalogs of this Invention

The established synthesis of illudalogs IA-3-IA-10 is outlined and discussed above (cf. FIG. 8). New illudalogs will be prepared similarly starting from 10 commercially available 2-bromobenzoic acid derivatives (5, X=OEt, Cl, etc., FIG. 10). Naphthalene substitution will be explored systematically by moving methyl and methoxy groups around the ring, along with R²-bromo and R¹⁻³-trifluoro derivatives. (These halides are expected to be inert to the benzannulation sequence. If not, then alternatives will be incorporated into the synthesis plans in their stead.) Claisen-type condensation with the lithium enolate of Weinreb acetamide will provide β-keto amides 1 for benzannulation with β-keto ester 2. The resulting naphthols (3) will be treated with lithium aluminum hydride, then strong or mild aqueous acid, alternatively. Strong acid is expected to promote global hydrolysis to provide illudalogs IA-11-IA-29 (odd numbers, FIG. 10). Mild acid will release aldehydes 4 with the acetal and ester still intact; subsequent phenol methylation followed by strong acid will provide illudalog methyl ethers IA-12-IA-30 (even numbers). These 28 illudalogs, along with illudalic acid (IA-1), ether IA-2, and 7-BIA^{14,18} as positive controls, comprise the first batch of illudalogs for pharmacological assessment. This first batch is focused on the naphthalene series, although indanes IA-1-IA-4 are included.

The second batch of illudalogs is proposed to probe the SAR of phenol alkylation. Naphthol aldehydes 4 will be converted into diverse phenol alkyl ethers. For example, the simple combinatorial synthesis pairing of five naphthols and one indane (e.g., the aldehyde precursor to IA-4, not shown) with five alkylating agents produces 30 illudalogs. The five alkylating agents identified in FIG. 10 (inset box) systematically vary steric profiles and create opportunities for further diversification (via the propargyl alkyne and/or carboxylate groups). The five naphthols include electronically neutral (IA-31-IA-35), rich (IA-36-IA-40), and deficient (IA-41-IA-45) arene cores; cores with a substituent proximal to the phenol (IA-46-IA-50), and brominated illudalogs (IA-51-IA-55, to facilitate X-ray crystallographic analysis; see below). The ether analogues of indane IA-4 are not illustrated here but will be designated IA-56-IA-60. Subsequent illudalogs will be designed based on the pharmacological data and observations from these first two rounds, with the third batch designated IA-61-IA-90. We prepare ≥90 diverse illudalogs.

### Expression of PTP Catalytic Domains

We maintain a library of PTP catalytic domains that have been purchased, from commercial sources or expressed and purified in the Barrios lab using plasmids obtained from various sources. The 21 human PTP catalytic domains (highlighted in FIG. 1) represent a diverse subset of the 38 classical, cysteine-dependent PTPs and include receptor-type PTPs, nonreceptor-type PTPs, and the low molecular weight PTP (LMWPTP). We maintain additional phosphatases for counterscreening, including the dual-specificity PTP VHR, alkaline phosphatase, acid phosphatase, and serine/threonine phosphatases. The activity of each of these enzymes is validated using DiFMUP (difluoromethylumbelliferyl phosphate, a commercially available fluorogenic phosphatase substrate).⁴²

### Initial Inhibitor Screen Assays

Compounds of this invention are tested at a fixed concentration against a panel of PTPs, as illustrated in FIGS. 7¹⁵ and 9. Enzyme activity assays are performed in black 96-well plates containing a total volume of 100 μL in each well. The experimental conditions are generally 5-10 nM of enzyme, 30-50 μM DiFMUP and 100 μM inhibitor. Prior to each assay, the enzyme is activated by incubating in Bis-Tris buffer (50 mM Bis-Tris pH 6.5, 100 mM NaCl, 0.01% Brij 35, 1 mM EDTA) with 5 μL of 100 mM TCEP on ice for 30 min. Buffer, enzyme, and either a DMSO control or the compound of interest are added to each well of the 96-well plate and incubated for 30 min at room temperature. After this 30-min preincubation period, the substrate DiFMUP is added to initiate the reaction. The observed fluorescence resulting from the hydrolysis of DiFMUP is measured every 60 s for 30 min in a SpectraMax M5 plate reader with excitation and emission wavelengths of 350 and 455 nm, respectively. The most promising compounds are then taken forward for more detailed kinetic analysis.

Inhibitor pKₐ and pH-Dependence Studies

To explore the effect of pH and pKₐ on inhibitory activity, we will repeat the initial inhibitory screen assays as outlined above at pH 7, pH 7.5, and pH 8, and measure inhibitor pKₐ⁴³ for select inhibitors in cases where a strong pH-dependence is observed. In our preliminary assays, we observed increased potency and selectivity for PTPR^{δ} at higher pH (cf. FIG. 9). Therefore, we expect that lowering the inhibitor pKₐ will likewise increase potency and selectivity for PTPR^{δ}. In particular, we will compare the proposed trifluoro-illudalogs (IA-29, IA-30, and IA-41-45) with IA-5, IA-6, and IA-31-35, based on the hypothesis that the trifluoro-illudalogs will show increased potency and selectivity for PTPR^{δ}. These compounds will also be examined for cell permeability and intracellular activity.

### IC₅₀ Curve and Time-Dependent Inhibition Assays

Assays to determine the IC₅₀ values for each hit compound are carried out as described in the initial inhibitory screen assays section using varying concentrations of inhibitor (generally 10 nM-100 μM). Assays to investigate time-dependent inhibition are performed as described in the initial inhibitory screen assays section with slight modifications. Buffer and enzyme are initially added to each well, followed by sequential addition of inhibitor starting from the longest incubation time to the shortest. Once all inhibitor has been preincubated for the desired time, assays are initiated by addition of DiFMUP to each well and the increase in fluorescence monitored as above.

K₁ and k_{inact}=Assays

Assays to determine k_{obs} values were performed as described in the Initial inhibitory screen assays section above with slight modification. Buffer, inhibitor and DMSO were added to each well as appropriate. Enzyme was then added to each well and allowed to incubate for the desired time, followed by addition of DiFMUP to initiate the assay. Varying concentrations of inhibitor (generally 1-50 μM) were incubated for 5-120 min. The k_{obs} values were obtained by fitting reaction progress curves as described previously, and then plotted against inhibitor concentrations to obtain K₁ and k_{inact} values.^{15,44} We expect our lead compounds to have improved K₁ and k_{inact} values as compared to illudalic acid and will work to identify compounds with K₁ values below 10 μM and k_{inact} values faster than 1 per min. Stopped flow kinetics instrumentation is available and can be used for these experiments as needed.

### Reversibility of Inhibition and Site(s) of Covalent Adducts

We will investigate the reversibility of the interaction between the illudalic acid analogues and the LAR-PTPs by subjecting the inhibited enzyme to dialysis against the reaction buffer 5× and monitoring for any recovery of activity. The site(s) of enzyme labeling will be identified by LC-MS of the labeled enzyme. Interestingly, our preliminary experiments with illudalic acid and LAR indicate that enzyme activity cannot be recovered by dialysis, but the expected adducts are not observed by LC-MS, consistent with pseudo-irreversible inhibition. We will repeat and optimize these experiments. Briefly, ˜2 nmol of enzyme will be concentrated to 0.5 mg/mL via MicroSep 10K columns. The inhibitor (10 mM stock solution in DMSO) will be titrated in 2 μL aliquots into the enzyme solution, and the enzyme-inhibitor mix will be allowed to incubate for 1 h. An aliquot of the enzyme-inhibitor mix will be monitored for enzyme activity using DiFMUP as a substrate and compared with the activity of a positive control of enzyme mixed with 2 μL of DMSO and incubated in parallel. Additional aliquots of inhibitor (or DMSO, for the positive control) will be added and incubated as necessary until the enzyme activity is below 5% of the control. Once enzyme is fully inhibited, all unreacted thiols will be reduced with 10 μL of a freshly prepared, 85 mM solution of DTT in 50 mM AmBic and incubated for 40 min on a shaker. The unreacted thiols will be alkylated with iodoacetamide (35 μL of a 55 mM solution in 50 mM AmBic, again freshly prepared) via incubation in the dark for 30 min with shaking. Excess iodoacetamide will be quenched via addition of 15 μL of the DTT solution and the proteins will be desalted and washed using MicroSep 10K columns. Aliquots will be taken for MS analysis of the intact protein and the rest of the protein will be subjected to trypsin digestion and an LC-MS analysis. By labeling the free cysteine residues in the protein with iodoacetamide, we will be able to identify the site of adduct formation even if the adduct is unstable under LC-MS conditions.

Co-Crystallization of LAR with Illudalic Acid

Expressed and purified LAR phosphatase will be mixed with illudalic acid or analogue and allowed to react under conditions established in the pharmacological assays described above. Following association, the enzyme complex will be concentrated and screened for initial solubility in both ammonium sulfate and PEG 4000 to establish a concentration threshold likely to promote nucleation in initial crystallization trials. Illudalic acid-LAR phosphatase complexes will be screened in nL-volume experiments using a crystallography robot.⁴⁵ Initial screening will test 480 conditions, examining a wide range of concentrations of common crystallography precipitants over a pH range of 4-10. The postulated covalent attachment of illudalic acid to LAR phosphatase protein is expected to be a significant advantage in our co-crystallization experiments compared to traditional experiments. Structural studies of ligands bound to enzymes is often challenging due to the propensity of the ligand-protein complex to dissociate during crystallization. Covalent attachment is ideal, circumventing many of the early pitfalls of co-crystallization analysis.

### X-Ray Crystallographic Analysis of the Illudalic Acid-LAR Bound Complex.

Initial crystal hits will be mounted and flash-frozen in loops and cryo-shipping pucks compatible with high-throughput screening robots. Initial crystal hits obtained will be screened for diffraction resolution under general user proposal #62011 at sector 24 of the Advanced Photon Source synchrotron.⁴⁶ This beam-line is equipped with robotic mounting systems and a Dectris EIGER direct electron x-ray detector. X-ray datasets will be collected using vector scanning translation methods to minimize radiation damage. Initial phasing will be performed using molecular replacement with the previously solved protein²⁸ from the RSCB Protein Data Bank⁴⁷ as a search model, followed by refinement using Phenix to allow visualization of the bound ligand complex. Solving phases by molecular replacement alone can lead to model bias; we will guard against this possible bias by repeating the experiment with Se-Met-derived protein and/or with bromo-illudalog IA-19. The synchrotron energy will be tuned to the anomalous peak of either Se and Br to collect experimental phases.

### Molecular Dynamics Simulations

Computational docking and dynamic simulation experiments will guide efforts to map the molecular interactions responsible for selective inhibition of LAR-PTP family members with illudalic acid analogues. As described above, we expect the illudalogs to be active site-targeted pseudo-irreversible inhibitors of LAR-PTP activity. The actual binding site and mode of our top hits will be identified using a combination of kinetic and thermodynamic experiments and structural biology as described above, and the resulting data will inform our molecular modeling experiments. The crystal structures of the tandem phosphatase domains of human LAR²⁸ and the catalytic domain of the rat ortholog of PTPRδ⁴⁸ have been solved and will be used in our modeling efforts (with the rat ortholog converted to the human enzyme via simulation). In addition, we will use the crystal structures of CD45 (PDB 1YGR) and HePTP (1ZC0) as selectivity controls to validate our molecular simulations. As described above, both illudalic acid and the methyl ether inhibit LAR and PTPRδ activity, while only illudalic acid inhibited CD45 activity, and none of the compounds tested thus far inhibit HePTP activity significantly.

We have a detailed SAR profile for illudalic acid-based LAR-PTP inhibition based on ca. 90 illudalogs and any active synthetic intermediates (e.g., 4), X-ray structural information of several (at least 2-4) bound complexes, and working mechanistic models of molecular recognition and ligation events supported by thermodynamic, kinetic, and crystallographic data and computational modeling. This detailed in vitro pharmacology will reveal how the general size and chemical nature of substituents on the tricyclic illudalog core structure influence potency and selectivity within 3 and beyond the LAR-PTP subfamily, with the PTP panel screening providing preliminary SAR information to guide future design and synthesis of selective inhibitors for other phosphatases. Any of the proposed illudalogs that prove elusive to synthesis would be replaced by others to similar effect and/or revisited later based on methodology evolving in parallel under Aim 3. We expect our novel illudalogs to have improved K₁ and k_{inact} values as compared to illudalic acid and 7-BIA, which will serve as positive controls and key benchmarks in our studies. We will work to identify compounds with K₁<10 μM and k_{inact}>1 min-. Novel illudalogs thus prepared and characterized serve as potential lead compounds for drug discovery.

### Identify and Characterize Intracellular Activity of Illudalic Acid-Based LAR-PTP Inhibitors

We establish that the proposed illudalogs are cell-permeable and capable of inhibiting LAR-PTP activity in vivo with minimal off-target interactions. We carry out a series of cellular experiments aimed at investigating the potency and selectivity of unmodified inhibitors and chemical probes. We use unmodified inhibitors to validate activity, and we develop and apply clickable probes to pull down the cellular target(s) of our illudalogs. As an example, initial experiments to validate the cellular activity of PTPRδ inhibitors will be carried out using primary cultures of cortical neurons and astrocytes from embryonic CF-1 mice. PTPRδ is highly expressed in many neuronal cell types,^{8b} and it dephosphorylates pY505 of STAT3, one of its key biological substrates.⁴⁹ Inhibition of cellular PTPR^{δ} activity is expected to result in an increase in STAT3 phosphorylation, which will be monitored via Western blot using a commercially available anti-pY705 STAT3 antibody. LAR and PTPR^{δ} are also expressed in neurons; we will carry out similar experiments to validate the cellular activity of LAR and PTPR¹ inhibitors using validated LAR and PTPR,^{δ} substrates.⁵ LAR is also highly expressed in several cancer cell lines.⁵⁰ We can use LAR-mediated dephosphorylation of beta-catenin in PC12 tumor cells as a further validation of our LAR inhibitors.⁵¹ By testing our illudalog inhibitors at varying concentrations in the cells, we can estimate cellular potency and compare it with the in vitro potencies.

In addition to validating unmodified inhibitors in cellular assays, we will develop chemical probes to identify the cellular targets of our illudalog inhibitors. For example, although an increase in STAT3 Y705 phosphorylation would be consistent with cellular PTPR^{δ} inhibition, other tyrosine phosphatases can also dephosphorylate this substrate.^{49b} To identify the specific illudalog cellular targets, we will synthesize illudalogs with an alkyne or azide for click functionalization with biotin tags for pull-down assays of labeled proteins.⁵² We will design our probes such that the azide or alkyne moiety does not interfere with enzyme inhibition (see examples in FIG. 11), validate their activity in inhibitory assays, and investigate any perturbation in a detailed kinetic analysis. The labeled probes are expected to have the same kinetic parameters as the parent inhibitors. The probes will then be used in cell culture and cell lysate to identify their biological target(s). The azide-modified probe will be useful in cell lysates and the alkyne-modified illudalog is expected to provide better selectivity and sensitivity for click applications in intact cells.^{2b}

### Cellular Activity of Illudalic Acid Analogues.

Primary cultures of cortical neurons and astrocytes will be prepared from embryonic CF-1 mice (Charles River, Wilmington, Mass.), as described previously.⁵³ Embryos (Day 14-15) will be removed from anesthetized CF-1 mice and the brains quickly removed from the embryos. Dissected cortical hemispheres will be gently chopped into small pieces and then incubated for 2 min in 15 mL DMEM with 0.25% trypsin. Then, 2 mL of heat inactivated horse serum (Invitrogen) will be added, and this mixture will be centrifuged for 2 min at 1800 RPM. The supernatant will be removed and the brains triturated and then spun again for 2 min at 1800 RPM. Cells will be re-suspended in 5 mL of Dulbecco's modified Eagle's medium supplemented with 10% horse serum, 10% fetal calf serum, 3% glucose, and 2% L-glutamine, and strained through a 70 μm cell strainer (BD Falcon). Cells will be plated at a high density of 500,000 cells/mL on poly-L-lysine (Sigma) coated six-well plates. Cultures will be maintained in a humidified incubator at 37° C. and 5% CO₂. When cells are ca. 70% confluent, the cultures will be treated with Ara-C(Sigma), 10-14 μL of a 1 μM stock, usually between 5 to 6 days after plating, to inhibit astrocyte growth. Media is replaced every 3-4 days, and experiments will be performed between 10-14 days in culture. Western blots will be performed as described by Geisler.¹⁴ Cells will be incubated with several different concentrations of compound (1, 10, 50 μM, depending on potency) or DMSO as a control for 30-120 min. The cells will then be lysed in a buffer containing EDTA and a cocktail of enzyme inhibitors^{38b} prior to Western blot analysis.

### Synthesis of Derivatized Probes

Alkyne IA-91, azide IA-92, biotin-tagged derivatives (e.g., IA-93-IA-97), and their corresponding methyl ethers (IA-98-IA-104, not shown) are prepared for use as chemical probes (FIG. 11). Many biotinylating reagents for click couplings are available; we will start with tetra(ethylene glycol) (PEG4) linkers by analogy to our previous collaborative efforts.⁵⁵ Many methods are available for making aryl alkynes or azides; discussion here highlights methods that align closely with our broader synthetic plans. For example, bromo-illudalog IA-19 (cf. FIG. 10) will be prepared by analogy to chloro-illudalog IA-8 (see FIG. 8). Sonogashira coupling of bromide IA-19 with TMS-ethyne (then desilylation) or a biotin-PEG4-alkyne produces IA-91 and IA-93. Bromide-to-azide substitution (to IA-92) can be achieved under mild Cu₂O-catalyzed conditions.⁵⁶ We expect that the illudalic acid pharmacophore will be compatible with these couplings; in fact, Sonogashira and other Pd-catalyzed couplings can be performed in vivo.⁵⁷ There is ample literature support for the viability of the proposed bromide couplings, but we could replace the bromides with iodides and/or triflates in our synthetic planning if milder coupling conditions are desired. Alternatively, we can introduce the alkyne or azide earlier (i.e., prior to unveiling the pharmacophore) and/or at different substituent locations on the illudalog, including as a propargyl ether on the phenol (cf. IA-34, IA-39, etc., FIG. 10 inset box). Attachment of biotin will be done in vitro (e.g., to IA-93), ex vivo (cell lysates; e.g., to IA-94 or IA-95), and/or in live cells (e.g., to IA-96 or IA-97) using biorthogonal reactions.⁵²

### Validation of Derivatized Probes

Prior to cellular experiments, we validate our probes with purified enzyme to ensure that derivatization does not significantly change the kinetic parameters of enzyme inhibition and to demonstrate that enzyme-bound probe can be labeled with our biotin reagents and imaged using SDS-PAGE. To label the enzyme-bound probe, enzyme is incubated with probe, followed by addition of excess labeling reagent. After sufficient labeling time (as indicated by the commercial supplier or literature reference for the probe), the labeled enzyme will be run on SDS-PAGE and the labeled band observed using an anti-biotin antibody. We expect to visualize labeling with each probe on a gel. We will test several different concentrations of the labeling reagent (and probe, if necessary) in order to obtain optimal labeling. The optimized conditions will then be used in the cellular experiments described below.

### Investigation of Probe Selectivity in Cells

To determine how selective our probes are for the enzyme of interest, and to identify other proteins that may be labeled by the probes in a cell, we incubate the cells with our azide-labeled probe at the optimal concentration and timepoint identified in the experiments described above in the cellular activity of illudalic acid analogues section. After incubation, the cells will be lysed in a buffer containing EDTA and an enzyme inhibitor cocktail as described above. The lysates will be incubated with the biotin labeling reagent using the conditions worked out previously and run on an SDS-PAGE gel, after which, bands containing the biotin label will be identified using an anti-biotin blot. We will validate PTPR^{δ} as one of the labeled proteins via Western blot with a PTPR^{δ} antibody.

We expect to demonstrate dose-dependent cellular inhibition of LAR-PTPs. Bicyclic analogues of illudalic acid have been used previously in cellular and animal studies,¹⁴ so we do not expect significant toxicity or challenges with cell permeability. It is possible that some of our analogues may have decreased cell permeability or increased toxicity, so we will monitor for these possibilities. We anticipate that the illudalogs will label cellular LAR-PTPs with some selectivity. In the event that other major targets are identified in our cellular experiments, we will further investigate the interaction between the inhibitor and newly identified target, and we will add the new target to our counter-screening panel for future rounds of inhibitor optimization, as appropriate. Finally, if our initial selection of conjugation chemistries does not provide sufficient labeling for the cellular experiments, we would investigate alternative bioorthogonal labeling tools.

We provide versatile and scalable syntheses of illudalic acid and analogues. The validated benzannulation approach to illudalogs outlined in the Preliminary Results and creates the opportunity of a 5-step (LLS) solution to the ≥16-step challenge of the total synthesis of illudalic acid itself. Natural product synthesis is a rigorous and uncompromising pursuit that advances chemical synthesis technology in ways that drive innovation in medicinal chemistry and molecular biology. Methodology here will focus on the benzannulation, subsequent reductive unmasking of the trifunctional pharmacophore, and phenol alkylation and substitution processes.

### Synthesis of Illudalic Acid

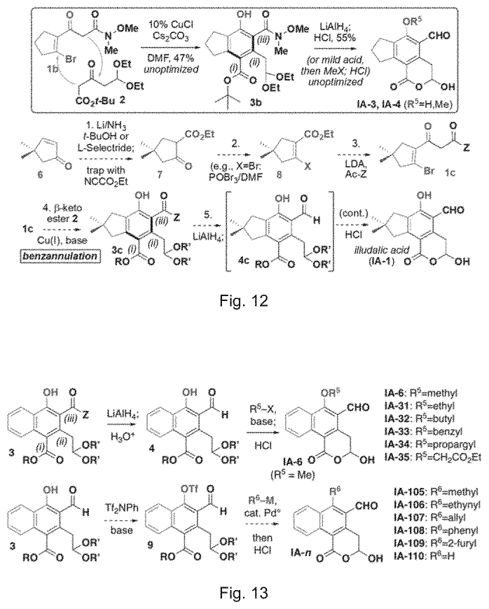

Our preliminary results establish that the two-step process of benzannulation and reductive unmasking of the pharmacophore is viable for indane-based illudalogs (FIG. 12, top; cf. FIG. 8). Here it will be applied and optimized for illudalic acid itself. We began investigations in the indane series with didesmethyl-illudalog IA-3 for two reasons. First, we have illudalic acid and immediate synthetic precursors already in inventory from prior work¹⁵; targeting IA-3 gave us a new compound to test. Second, this model study began with cyclopentanone, a convenient starting material for preparing β-keto amide 1b. In contrast, 4,4-dimethyl-cyclopentenone (6) is available from from Sigma-Aldrich, and it is not hard to make,⁵⁸ and diverse γ-alkylated cyclic enones are available.⁵⁹

For the proposed synthesis of illudalic acid (FIG. 12, bottom), we will start with reductive C-acylation⁶⁰ and bromidation to prepare enoate 8 (X═Br), then Claisen condensation with Weinreb acetamide to produce β-keto amide 1c. Benzannulation and reductive unmasking of the pharmacophore complete the 5-step (LLS) synthesis of illudalic acid. We chose the (i) tert-butyl ester, (ii) formylmethyl diethyl acetal, and (iii) Weinreb amide in our initial studies to facilitate selective reduction and hydrolysis to the trifunctional pharmacophore, but these choices are not optimized. For example, bulky groups compromise the benzannulation efficiency; best yields in the original methodology³⁰ were with smaller groups (e.g., methyl and benzyl esters and a methyl group). We chose the i-butyl ester to see if it would withstand the conditions for phenol-directed partial reduction of the Weinreb amide (which it did) and to allow for mild acidic hydrolysis, but strong aqueous acid has proved advantageous thus far. Simple esters should suffice. Therefore, we will examine smaller, simpler, and/or more robust groups: (i) methyl or benzyl ester, (ii) formylmethyl dimethyl acetal or ethylene glycol acetal, and (iii) morpholine amide or nitrile. The optimal selections will maximize the combined yield of steps 4 and 5 without compromising other metrics for the overall process. Iodides and triflates will also be examined for the benzannulation (cf. 8, X=1, OTf).

Phenol derivatives. Based on preliminary data, methylation of the phenols impacts selectivity within the LAR-PTPs, especially for PTPR^{δ}. We have been making illudalog methyl ethers as outlined in FIG. 13 (top, R⁵=Me). In the current process, hydride reduction of Weinreb amides 3 with mild acidic workup provides salicylaldehydes 4, which are methylated using NaH and Me2SO₄. Methylation yields are modest (ca. 20-40%) but sufficient for preparing the diverse illudalogs proposed above for pharmacology and SAR studies.

We focus on process optimization in the preparation of illudalog ethers. We optimize phenol alkylation by varying the base, solvent, and other typical reaction parameters, along with the alkylating agent (FIG. 13, top). We will then expand the scope of illudalogs to ones prepared by substitution of the phenol (FIG. 13, bottom). To this end, the phenol will be converted into the aryl triflate for metal-catalyzed cross-coupling reactions to introduce various alkyl and aryl substituents at this position, as well as simple hydrogen by reductive cleavage. Although the triflate is hindered, electron-withdrawing groups at the ortho- and para-positions are often advantageous for such couplings. Conversion of the phenol to the aryl fluoride, acetate, carbonate, azide, and aniline will also be examined; the fluoride and aniline derivatives are of particular interest to us from an SAR perspective and for exploratory investigations into PTP selectivities,

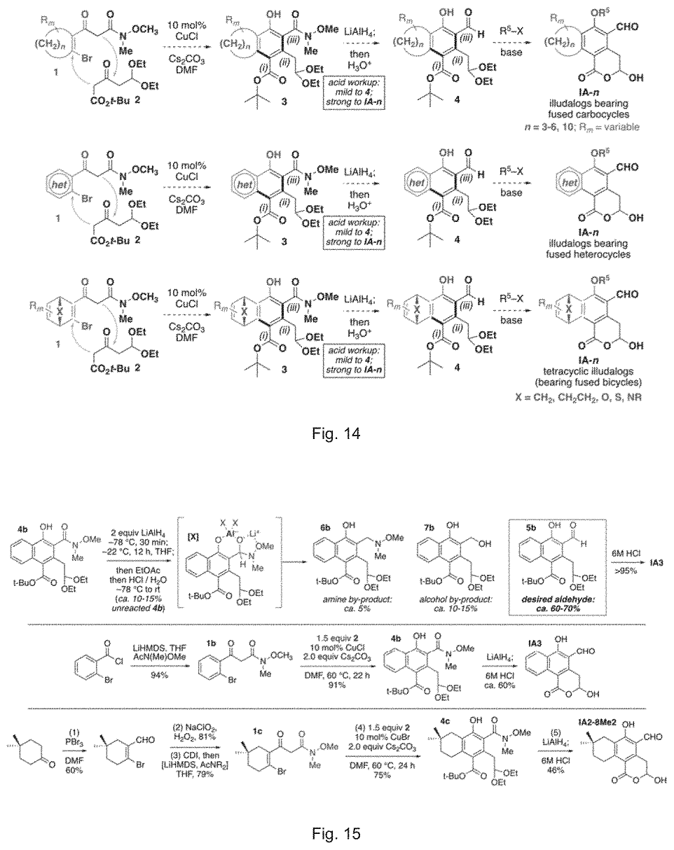

Multigram-scale synthesis. We will validate the optimized process with multigram-scale syntheses of illudalic acid and at least one naphthalene-based illudalog ether derivative (e.g., IA-6). Demonstration of multigram synthesis capabilities will be important for planning future in vivo pharmacology experiments in animals. We then focus our attention on the versatility of the synthetic process with an eye toward incorporation of novel scaffolds.
Validation of diverse scaffolds. The illudalic acid pharmacophore merits exploration beyond the indane- and naphthalene-based systems. For future design and synthesis of selective inhibitors, including to target other classical PTPs, we propose to examine alternative scaffolds for the illudalic acid pharmacophore. Specifically, we aim to validate the versatility of our benzannulation approach by annealing the pharmacophore onto larger carbocycles, heterocycles, and bridged bicycles (see FIG. 14). All new compounds will be evaluated for PTP activity.

### Synthesis and Validation of Diverse Scaffolds

The established synthesis of illudalogs IA-3-IA-10 is outlined and discussed above (cf. FIG. 8). The illudalic acid pharmacophore merits exploration beyond the indane- and naphthalene-based systems exemplified there. For future design and synthesis of selective inhibitors, including to target other classical PTPs, we examine alternative scaffolds for the illudalic acid pharmacophore. Specifically, we validate the versatility of our benzannulation approach by annealing the pharmacophore onto larger carbocycles, heterocycles, and bridged bicycles (FIG. 14). These syntheses will follow the same general plan, starting with β-keto amides 1 and β-keto ester 2. For illudalogs bearing fused carbocycles, we envision starting with various cycloalkanones, α-acylating with Mander's by analogy to FIG. 12, and carrying with the rest of the benzannulation and functional group interconversions as outlined previously. Synthesis of illudalogs bearing fused hetercycles will be examined by analogy to FIG. 10 but starting with 2-bromocarboxylic acid derivatives of various heterocycles (e.g., pyridine, pyrazine, etc.). Alternative methods for preparing these heterocyclic variants of 1 are undertaken. For tetracyclic illudalogs, we will start with bicyclic β-keto amides 1, derived from the corresponding bromocarboxylic acid derivatives. The compounds of this invention that are exemplified in FIG. 14 are prepared by Diels-Alder reaction between 3-bromopropynoates and cyclopentadiene, cyclohexadiene, furan, thiophene, or pyrrole derivatives (for X═CH₂, CH₂CH₂, O, S, or NR, respectively). Other tetracyclic (and larger) illudalogs can likewise be prepared and are compounds of this invention.

The present invention provides an efficient, versatile, and scalable 4-5-step synthesis of illudalic acid that enables production of the natural product and/or diverse designer analogues for chemotherapeutic discovery and development. In contrast, the previous syntheses of illudalic acid require 16-20 steps, and previous analogue designs focused on making the synthesis easier rather than improving pharmacological efficacy. We set forth other indane benzannulation strategies and tactics as needed for developing a concise and robust gram-scale synthesis of illudalic acid. The present invention includes indane, naphthalene core systems. One limitation is the cost of cyclopentenone 6. This cost may be reduced by making it ourselves⁵⁸ or developing an alternative route to β-keto amide 1c, which would also provide an opportunity to vary substituents on the fused cyclopentane in the design and synthesis of next-generation analogues.

Another limitation is that the proposed synthesis necessarily delivers the indane phenol functional group. The phenol is found in the natural product, so this is not a limitation for the natural product synthesis per se, but it would have to be blocked or removed if it turns out not to be optimal for pharmacological efficacy in future analogues. Finally, as noted above, new compounds prepared are evaluated for PTP inhibitory activity.

Of particular interest are applications of PTPR^{δ} inhibitors to investigate the role(s) of PTPR^{δ} in the development and treatment of stimulant addiction in rat models of cocaine self-administration. PTP panel screens provide preliminary SAR information. Other functional derivatives (fluorogenic probes, chimeras, prodrugs, etc) and alternative scaffolds (perhaps to target other cysteine-mediated enzymatic processes) for the illudalic acid pharmacophore are example compounds of this invention. In summary, the present invention will advance natural products synthesis; provide blueprints for making libraries and/or multi-gram quantities of LAR-PTP inhibitors; and begin to establish a general methodology for the design and synthesis of selective phosphatase inhibitors for chemical biology, biomedical research, and drug discovery.

### Synthesis Scheme

The synthesis of our two key building blocks is outlined in Scheme 1 below. Pinnick oxidation of known bromo enal 5 (prepared here by Vilsmeier-Haak-type formylation of 3,3-dimethylcyclopentanone) set up CDI-mediated Claisen-type condensation to provide β-keto amide 3a. A similar CDI-mediated decarboxylative coupling of acids 6 and 7 produced β-keto ester 4 in 72% yield.

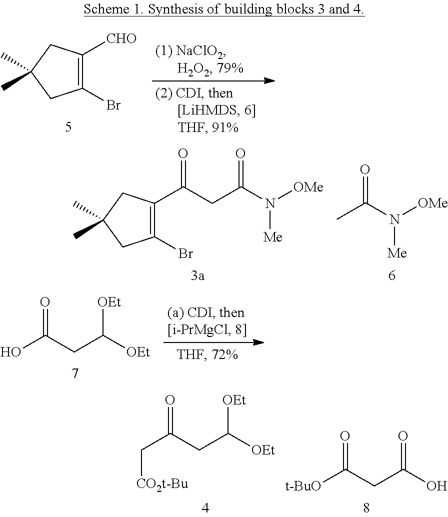

The Cu-catalyzed, base-mediated [4+2] benzannulation of β-keto amide 3a with β-keto ester 4 provided indane 9a in 79% yield (Scheme 2-below). Excess β-keto ester 4 is recoverable by chromatography and can be recycled. The mechanistic pathway outlined in Scheme 2 is consistent with and our observations: Cu-catalyzed coupling of enolate Cs·4 with vinyl bromide 3a triggers aldol cyclization to produce cyclohexenol intermediate 10, which persists in solution. Dehydration of 10 can produce either of two isomeric cyclohexadienones (not shown) but is slow, presumably because A-strain disfavors formation of enol tautomers of 10. Excess Cs₂CO₃ is advantageous for promoting dehydration, whereas less Cs₂CO₃ and/or other bases were less effective.

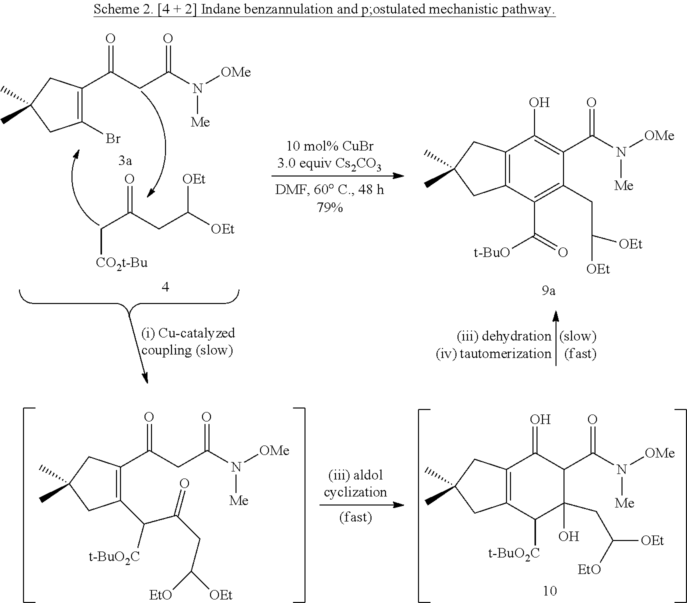

Homocoupling of β-keto amide 3a occurs in the absence of β-keto ester 4 but is effectively suppressed by the desired benzannulation. Deprotonation of ester 4 is presumably more facile under the mildly basic conditions compared to amide 3a, favoring the desired coupling. Replacing the Weinreb amide of 3a with ester or nitrile functionality here was deleterious to the reaction outcome, although we have not thoroughly explored these alternatives.

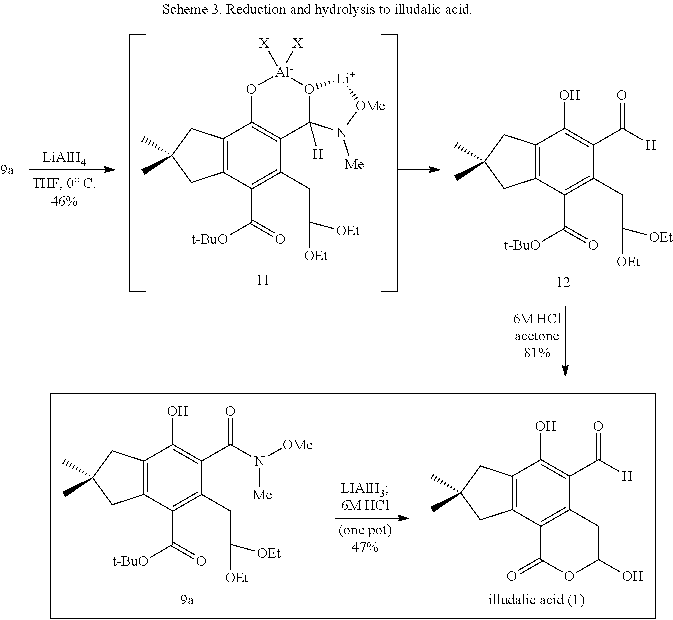

Selective reduction of the Weinreb amide can be accomplished with LiAlH₄ in THF (Scheme 3). Chemoselectivity for reduction of the amide over the ester was our largest concern at the outset, but in practice the greater difficulty was in stopping reduction at the aldehyde stage. The phenol of 9a is positioned to direct hydride reduction of the amide, but phenoxide intermediate 11 is prone to decomposition in situ, which leads to over-reduced byproducts. Dropwise addition of LiAlH₄ to a solution of 9a in THF at 0° C. immediately produces bubbles, presumably of hydrogen gas, which subside before a full equivalent of LiAlH₄ is added. Reduction of the amide is complete within 1 h (hour), producing aldehyde 12 as the major product (46%) along with alcohol and amine by-products from overreduction of the amine. Reduction of the i-butyl ester was not observed. Hydrolysis of the diethyl acetal and t-butyl ester can be accomplished simultaneously with 6M aqueous HCl in acetone, producing illudalic acid (1) in 81% yield.

Alternatively, acidic hydrolysis can be incorporated into the workup of the Weinreb amide reduction step. Thus, reduction of indane 9a with LiAlH₄ followed by workup with strong acid produces illudalic acid in 47% yield from 9a, or 27% over four steps from enal 5.

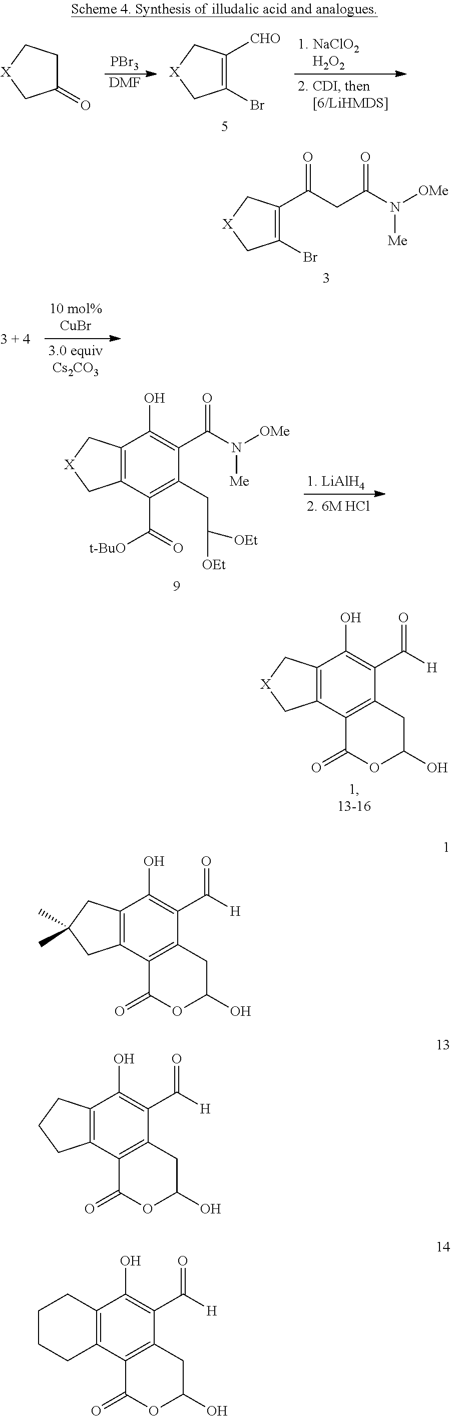

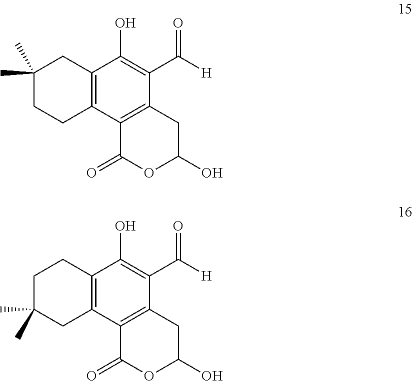

Alternative β-keto amides 3 provide access to novel tricyclic analogues of illudalic acid (Scheme 4), with β-keto ester 4 serving as a common building block. Bromo enals 5 are available by Vilsmeier-Haack-type formylation of cyclic ketones. Oxidation and coupling with Weinreb acetamide (6, cf. Scheme 1) provides β-keto amides 3. Once amides 3 are prepared separately, parallel benzannulations with β-keto ester 4 furnish indanes and tetralins 9. Reduction and acidic hydrolysis complete the assembly of analogues 13-16. Compounds 13-16 of this invention are potent and selective for the LAR-PTP subfamily (LAR, PTPRσ, and PTPR^{δ} and selected receptor-type and non-receptor-type PTPs (PTP1B, SHP2, and CD45). Compound 15 appears to be most potent and is selective for the LAR subfamily, its >50% inhibition of SHP2 at 250 nM places compound 15 in league with some of the most potent leading SHP2 inhibitors known. Compound 16 shows a different selectivity profile within the LAR subfamily, which provides molecular design for reversing selectivity between PTPR^{δ} and LAR. Thus those persons skilled in the art will understand that the compound s of tis invention are potent, selective, and covalent inhibitors of the LAR subfamily of tyrosine phosphatase enzymes and are lead compounds for therapy.

### New Synthesis of Illudalic Acid and Illudalogs

As discussed above, our new synthetic approach focuses on convergent benzannulation, followed by conversion to the ortho-formyl hydroxy-lactone pharmacophore (FIG. 1). FIG. 1 shows several examples of the structures of the illudalic acid analogues of this invention, and more specifically shows convergent benzannulation (unoptimized) for the synthesis of illudalic acid and illudalog compounds of this invention, (a) synthesis of b-keto amides 1; synthesis of β-keto ester 2 (inset box); (b) benzannulation: 10 mol % CuCl or CuBr, 2 equiv Cs₂CO₃, DMF, ca. 50-90% yields; (c) 2 equiv. LiAlH₄, then 6M HCl, ca. 40-70% yields of IAn compounds, which generally precipitate from aqueous acid as white crystalline solids. Intermediates 5 can be isolated for derivatization prior to acidic hydrolysis or advanced directly to the IAn compounds. Optimization is proposed and in progress. Currently, IA1 is produced in ca. 27% yield over 4 steps from 3. We have prepared >35 g of ester 2; ≥100 mg of IA1 and IA3; and ≥10 mg each of the other illudalog compounds of this invention. We have validated this approach for making illudalic acid (IA1) and >25 illudalogs (cf. FIG. 5) in as few as 2 steps from β-keto amides 1 and β-keto ester 2. For IA1, we access β-keto amide 1a in two steps from known enal 3.⁶¹ Other β-keto amides 1 provide parallel access to diverse illudalog cores (e.g., 1b→IA3, FIG. 1, bottom). We prepare β-keto ester 2⁶² on multigram-scale using CDI-mediated decarboxylative coupling;⁶³ we have >35 g of 2 in stock. β-Keto ester 2 is the common building block for illudalogs prepared thus far; it comprises functional precursors to the hydroxy-lactone. The benzannulation is adapted from a known naphthol annulation.⁶⁴ The final reduction and hydrolysis sequence can be executed as a one-pot operation or with isolation of intermediate 5. Presumably, the deprotonated phenol directs reduction of the ortho-amide over the para-ester. For example, treatment of indane 4a or naphthol 4b with LiAlH₄ and then strong aqueous acid produces IA1 or IA3, respectively. Alternatively, milder workup releases o-hydroxy aldehydes 5 (FIG. 1, top) with the ester and acetal still intact, which is convenient for late-stage functionalization. In most cases, the illudalog precipitates after hydrolysis from the aqueous acid solution as a crystalline solid. A notable exception is that illudalic acid itself does not precipitate directly when prepared in one-pot from 4a, but it can be crystallized separately. This (unoptimized) approach is convenient and effective; we currently secure IA1 in 27% yield over 4 steps from 3. Whereas stability concerns with 7-BIA in aqueous DMSO have been noted, illudalic acid and the illudalog compounds of this invention have proven to be indefinitely stable to storage as pure substances or in DMSO. For example, an NMR sample of illudalic acid in DMSO was unchanged after storing at room temperature for six months. Thus, it will be understood by those persons skilled in the art that the structures of additional illudalog compounds of this invention are set forth in FIG. 5. The naphthalene IA-3 series, being easier to diversify than 1A-1 and the 1A-2 series indanes and tetralins (inset boxes of FIG. 5) based upon arene substitution chemistry.

We have prepared ≥100 mg of IA1 and IA3, and ≥10 mg of all other illudalog compounds of this invention listed in FIG. 5. Illudalogs are named based on their core scaffold and substitution pattern. For example, illudalic acid is IA1, and its phenol methyl ether is IA1-6OMe. The same ether of its 8,8-didesmethyl analog is IA1-6OMe8H2. The IA2 series comprises compounds with a tetralin core, and the IA3 series comprises naphthalenes. The naphthalene series is the easiest to make based on the availability of many 2-bromobenzoic acid derivatives. For example, with β-keto ester 2 in hand, we can make IA3 in three steps and ca. 50% overall yield from 2-bromobenzoyl chloride (cf. FIG. 1, bottom). Likewise, IA3-6OR derivatives were made by alkylating the aldehyde intermediate 5b (cf FIG. 15), followed by strong acid to deprotect and precipitate IA3-6OMe and its ethyl, propargyl, and benzyl variants. The 7-, 8-, and 9-methyl IA3 derivatives were prepared to probe structure space systematically. Other IA3-series illudalogs with methoxy(s), trifluoromethyl, various halides, and t-butyl substituents were made to examine stereoelectronic factors. Alkynes IA3-8Br and IA3-8C2H provide potential handles for future functionalization and probe development.

### Preliminary Screening for Inhibitory Selectivity and Potency

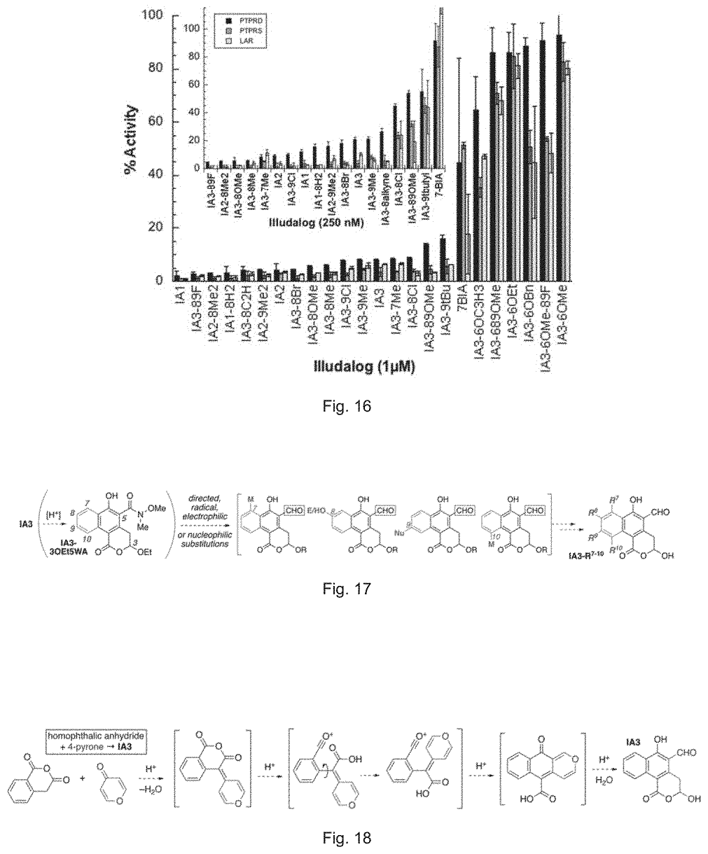

The inhibitory activity of illudalic acid increases with increasing pH and temperature. We reported an IA1 IC₅₀=2.1±0.2 μM for LAR at pH 6.5 and 22° C.,¹⁵ whereas others have recently remeasured an IA1 IC₅₀=52±11 nM for LAR at pH 7.5 and 37° C. The pH and temperature dependence of PTP inhibition is consistent with the postulated two-stage binding mechanism and covalent ligation kinetics. Based on this observation, they rescreened our inhibitors at 1 μM, pH 7.5, and 37° C. (as opposed to 100 μM, pH 6.5, and 22° C.). The illudalogs, like illudalic acid, are generally potent PTPRD inhibitors under these conditions (FIG. 16) and selective for the LAR/DSK subfamily PTPs (data not shown). Many are significantly more potent than 7-BIA. Alkyl ethers at the 6-position, like 7-BIA, provide lower inhibitory activity (e.g., IA3-6OMe, IA3-6OEt, etc.), with this difference being more pronounced at pH 7.5 and 37° C. than at pH 6.5 and 22° C. Additional screening of the most promising illudalogs at 250 nM under the same conditions (inset chart) reinforces evidence of their potency against the LAR/DSF subfamily of PTPs. IC₅₀ values for illudalic acid (IA1) and selected analogs against PTPRD at pH 7.5 and 37° C.:
7-BIA IC₅₀=2.0±1.6 μM (PTPRD), consistent with the prior estimate of 1-3 μM.¹⁴
IA1 IC₅₀=90±14 nM (PTPRD), showing the importance of the full tricyclic core and free phenol
IA3-89F IC₅₀=51±5 nM (PTPRD), perhaps suggesting increased potency with increasing acidity
IA2-8Me2 IC₅₀=45±+2 nM (PTPRD), suggesting increased potency with increasing hydrophobic bulk

The potency of IA3-89F and IA3-8Me2 is consistent with the observations that PTPRD has a preference for bulky, hydrophobic and/or acidic amino acid residues in the vicinity of its phosphotyrosine substrate.

Synthetic Chemistry of Illudalog Compounds of this Invention:

The synthetic chemistry of the illudalog compounds of this invention develops selective phosphatase inhibitors based on illudalic acid. The concise 2-step sequence of benzannulation and reduction/hydrolysis creates PTPRD-targeting therapeutics.

Optimize Reduction Sequence

Hydride reduction of the Weinreb amide (FIG. 15, top) is the low-yield step in the synthesis of IA3. We previously tested various hydride reagents and settled on lithium aluminum hydride (LiAlH₄) as the most convenient choice. The main undesired by-products in the reaction are methoxyamine 6b and alcohol 7b from over-reduction of the amide. These by-products emerge prior to complete consumption of starting material, and excess LiAlH₄ does not dramatically alter by-product formation. Given sufficient LiAlHₐ to consume the starting material, by-product formation arises from a combination of in situ breakdown of tetrahedral intermediate [X] and inefficient quenching. Specifically, thermal extrusion of aluminate anions at warmer reaction temperatures produces a methoxy-iminium, which is quickly reduced to 6b. Alcohol 7b may arise during the quenching process: if hydrolysis to the aldehyde occurs prior to complete consumption of excess hydride, then the aldehyde is instantly vulnerable to over-reduction.

Lower temperatures and/or less polar solvents will slow the extrusion of aluminate salts relative to the initial amide reduction, thereby suppressing formation of amine 6b. Low-temperature and/or aprotic hydride-quenching protocols will more effectively consume excess LiAlH₄ prior to hydrolysis of [X], thereby suppressing formation of alcohol 7b. The mass balance in these reactions is typically high (≥90%), distributed between desired aldehyde 5b (60-70%), by-products 6b (5%) and 7b (10-15%), and residual starting material (10-15%), which suggests that >80% yield of aldehyde 5b is an achievable goal for this reaction process.

Once a sufficiently optimized reduction protocol is established, we pair it with in situ acidic hydrolysis of the acetal and ester in a one-pot reduction/hydrolysis sequence from amide 4b to IA3. We then prepare multigram-quantities of IA3, e.g., by the 3-step sequence from commercially available 2-bromobenzoyl chloride shown in FIG. 15 (middle). We have prepared >35 grams of ester 2. Our 5-step route to IA2-8Me2 from 4,4-dimethylcyclohexanone (FIG. 15, bottom) having a promising potency (IC₅₀=45 nM).

Late-Stage Functionalization of IA3

IA3 and/or its presumably more robust variant IA3-3OEt5WA (FIG. 17) can serve as convenient starting points for diversification and derivatization. We will develop phenol-directed C—H functionalization⁶⁵ of position 7; phthaloyl peroxide-mediated hydroxylation⁶⁶ of position 8; nucleophilic C—H functionalization⁶⁷ of position 9; lactone-directed C—H hydroxylation⁶⁸ of position 10; and/or electrophilic aromatic substitution of positions 8 and/or 10. Additional substitution reactions can further functionalize this ring system.

We will test a hypothetical 1-step annulation of homophthalic anhydride and 4-pyrone, both of which are commercially available. We employ an acid-catalyzed sequence of condensation, rearrangement, and rehydration to IA3 (FIG. 18). Various acids (Brønsted and/or Lewis) and solvents (protic, polar aprotic, and nonpolar under aqueous or anhydrous conditions) will be examined. The fact that IA3 precipitates from 6N HCl in our current synthesis encourages us in this plan. If successful, then this 1-step route facilitates the late-stage functionalization activities, and we endeavor to expand this annulation.

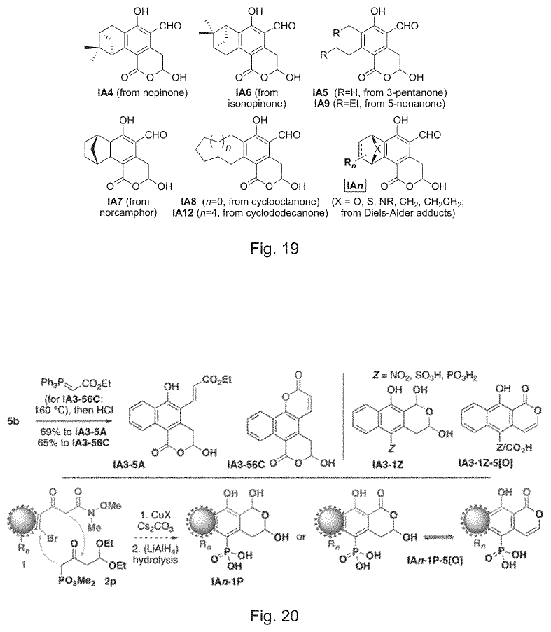

We make a series of hydrophobic illudalogs by analogy to previous syntheses of IA1 and IA2 (indane and tetralin) compounds (cf. FIG. 1 and FIG. 5). Our design hypothesis favoring larger, more hydrophobic illudalogs is based on two prior observations. First, PTPRD selects for phosphotyrosine residues that are close to large, hydrophobic, or acidic residues. Second, IA2-8Me2—having a slightly larger third ring—is more potent than illudalic acid against PTPRD (51 nM vs. 90 nM). We make hydrophobic illudalogs (IA4, IA5, etc, FIG. 19) from acyclic, cyclic, and bicyclic ketones. We then make a series of more elaborate and diverse illudalogs from Diels-Alder adducts of various dienophiles with 3-bromopropiolates, which provides myriad options for diversifying the core structure (cf. IAn), including with additional acidic substituents. Additional derivatives will be made for any IAn-series variants as will be appreciated by those persons of ordinary skill in the art. We made 70 mg of IA1 and then completed the synthesis of IA4 (55 mg; 17% overall from nopinone). We expect IA4 to be more potent than IA2 and IA2-9Me2.

### Experimental Chemistry

All the chemicals were used as received unless otherwise stated. Diethyl ether (Et₂O), tetrahydrofuran (THF) and methylene chloride (DCM) were dried over a column of molecular sieves under argon. All reactions were carried out under an inert nitrogen atmosphere unless otherwise stated. Crude products were purified in Biotage Isolera One Flash Purification System using Biotage prepacked cartridges (50 μm irregular silica). Yields refer to isolated material considered to be ≥95% pure following silica gel chromatography. ¹H-NMR and ³C-NMR spectra were recorded on a JEOL 400 MHz spectrometer using CDCl₃ and DMSO-d₆ as the deuterated solvent (≥99.8 atom % D, contains 0.03% (v/v) TMS). The chemical shifts (5) were reported in parts per million (ppm) relative to the internal standard TMS. High-resolution mass spectral (HRMS) data were obtained on a UHR-TOF maXis 4G instrument (Bruker Daltonics, Bremen, Germany) using electrospray ionization (ESI).

### Preparation of tert-butyl 5,5-diethoxy-3-oxopentanoate-(4)

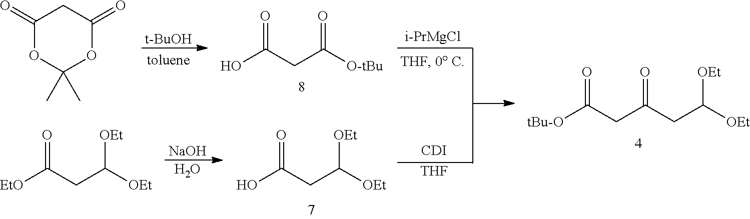

3,3-diethoxypropanoic acid-(7). A mixture of ethyl 3,3-diethoxypropanoate (10.0 g, 52.6 mmol, 1.0 equiv.), NaOH (7.15 g, 158 mmol, 3.0 equiv.) and water (1.0 M) was stirred at 70° C. for 3 hrs. After the reaction, the mixture was cooled in an ice bath, carefully acidified with concentrated HCl and extracted with diethyl ether. The combined organic extracts were washed with brine, dried over Na₂SO₄ and concentrated under reduced pressure to afford a colorless liquid which was used in the next step without purification (7.91 g, 93% yield). ¹H NMR (400 MHz, CDCl₃): δ 1.21 (t, 6H), 2.72 (d, 2H), 3.58 (dq, 2H), 3.69 (dq, 2H), 4.97 (t, 1H), 11.22 ppm (br s, 1H). ¹³C{¹H} NMR (100 MHz, CDCl₃): δ 15.19, 39.75, 62.09, 99.31, 175.83.

3-(tert-butoxy)-3-oxopropanoic acid-(8). To a solution of Meldrum's acid (10.58 g, 73.4 mmol, 1.0 equiv.) in toluene (1.25 M) was added tert-butanol (6.53 mL, 88.1 mmol, 1.2 equiv.). The solution was refluxed for 3 h, before being concentrated in vacuo. The viscous clear liquid was taken on to the next step without further purification (11.5 g, 71.8 mmol, 98%). ¹H NMR (400 MHz, CDCl₃): δ δ 1.49 (s, 9H), 3.36 (s, 2H), 11.41 ppm (s, 1H). ¹³C{¹H} NMR (100 MHz, CDCl₃): δ 27.93, 42.08, 83.00, 166.32, 172.24.

### tert-butyl 5,5-diethoxy-3-oxopentanoate-(4)

Mixture A. 1,1′-Carbonyldiimidazole (8.15 g, 50.3 mmol, 1.1 eq) was added in 3 portions to a solution of the acetal 7 (7.41 g, 45.7 mmol, 1.0 equiv.) and THF (0.2 M). The resulting mixture was stirred for 1 hr at room temperature.

Mixture B. To solution of the ester 8 (10.98 g, 68.5 mmol, 1.5 eq) in THF (0.5 M) at 0° C., isopropyl magnesium chloride (73.1 mL, 3.2 equiv., 2.0 M in THF) was added dropwise using a syringe pump set at a rate of 5 mL/min. The solution was continued to stir for an additional hour at room temperature.

Using a cannula, Mixture B was transferred to Mixture A. The creamy mixture was stirred at room temperature for 12-15 hrs. After the reaction, the mixture was quenched with saturated NH₄Cl, stirred for an additional 15 mins and extracted with ethyl acetate. The organic layer was washed brine, dried over Na₂SO₄, and concentrated under reduced pressure. The crude product was purified by automatic flash column chromatography on silica gel (gradient elution from 2% to 20% EtOAc-hexanes) to afford 4 as a light yellow, viscous liquid (72% yield, 93:7 mixture of keto and enol tautomers). ¹H NMR (400 MHz, CDCl₃): δ 1.20 (t, 6H), 1.47 (s, 9H), 2.85 (d, 2H), 3.41 (s, 2H), 3.54 (dq, 2H), 3.67 (dq, 2H), 4.89 ppm (t, 1H). ¹³C{¹H} NMR (100 MHz, CDCl₃): δ 15.26, 28.00, 47.61, 51.62, 62.51, 81.93, 99.77, 166.30, 200.63. HRMS (ESI, m/z) for C₁₃H₂₄O₅ [M−H]⁻: calcd, 259.1551; found, 259.1553.

### General Procedure for the Vilsmeier-Haack Formylation

Phosphorus tribromide (PBr₃, 2.2 equiv.) was added dropwise to a solution of DMF (3.0 equiv.) in DCM (1.0 M) previously cooled at 0° C. After 90 mins of stirring, the ketone (1.0 equiv.) was added neat. The white slurry was then warmed to ambient temperature and stirred for 60 hrs. After the reaction, the orange mixture was poured on crushed ice, neutralized with solid NaHCO₃ and extracted with DCM. The combined organic extracts were dried over NaSO₄, concentrated under reduced pressure and purified by automatic flash column chromatography on silica gel (step gradient: 3% then 5% EtOAc-hexanes).

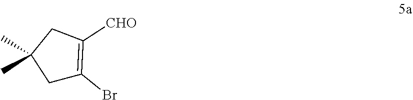

Obtained as colorless, volatile liquid (63%% yield). ¹H NMR (400 MHz, CDCl₃): δ 1.14 (s, 6H), 2.32 (t, 2H), 2.70 (t, 2H), 9.86 ppm (s, 1H). ¹³C{¹H} NMR (100 MHz, CDCl₃): δ 29.51, 37.68, 43.98, 56.95, 139.16, 139.65, 189.58.

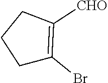

Obtained as colorless, volatile liquid (55% yield). ¹H NMR (400 MHz, CDCl₃): δ 2.03 (quint, 2H), 2.54 (tt, 2H), 2.91 (tt, 2H), 9.90 ppm (s, 1H). ¹³C{¹H} NMR (100 MHz, CDCl₃): δ 21.43, 29.32, 42.61, 140.04, 141.57, 189.30

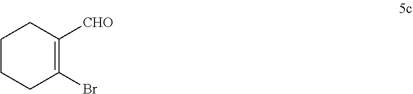

Obtained as colorless liquid (59% yield). ¹H NMR (400 MHz, CDCl₃): δ 1.65-1.73 (m, 2H), 1.74-1.81 (m, 2H), 2.28 (tt, 2H), 2.75 (tt, 2H), 10.02 ppm (s, 1H). ¹³C{¹H} NMR (100 MHz, CDCl₃): δ 21.19, 24.37, 25.10, 38.92, 135.36, 143.67,

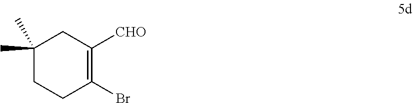

Obtained as colorless liquid (76% brsm). 1H NMR (400 MHz, CDCl₃): δ 0.95 (s, 6H), 1.52 (t, 2H), 2.08 (m, 2H), 2.76 (tt, 2H), 10.03 ppm (s, 1H). ¹³C{¹H}NMR (100 MHz, CDCl₃) S 27.68, 28.52, 28.53, 36.81, 38.34, 134.04, 142.72, 193.93.

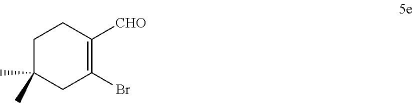

Obtained as colorless liquid (mixture of isomers, 93% 216 brsm;). ¹H NMR (400 MHz, CDCl₃): δ 0.98 (s, 6H), 1.47 (t, 2H), 2.31 (tt, 2H), 2.54 (t, 2H), 10.04 ppm (s, 1H). ¹³C{¹H} NMR (100 MHz, CDCl₃): δ 22.75, 27.89, 32.33, 33.90, 52.43, 134.03, 142.81, 193.75.

### General Procedure for the Pinnick Oxidation

A mixture containing the haloenal (5a-5e) (1.0 equiv), monopotassium phosphate (0.16 equiv.), acetonitrile (1.0 M) and 30% H₂O₂ (1.05 equiv.) was cooled to 0° C. and stirred for 15 mins. Sodium chlorite (1.5 equiv., 1.3 M in water) was then added and the mixture was stirred at ambient temperature for 5 hrs. After the reaction, the heterogenous mixture was acidified with 3M HCl. The solids were filtered, washed with cold water and air dried. The product was used in the next step without further purification.

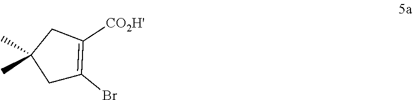

Obtained as white crystalline solid (79% yield). ¹H NMR (400 MHz, CDCl₃): δ 1.15 (s, 6H), 2.48 (s, 2H), 2.66 ppm (s, 2H). ¹³C{¹H} NMR (100 MHz, CDCl₃): δ 29.40, 37.32, 47.55, 57.83, 130.31, 133.98, 169.16. HRMS (ESI, m/z) for C₈H₁₂BrO₂ [M+H]⁺ calcd, 220.0049; found, 220.0048.

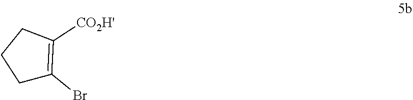

Obtained as white powdery solid (79% yield). ¹H NMR (400 MHz, CDCl₃): δ 2.00 (quint, 2H), 2.68 (tt, 2H), 2.86 ppm (tt, 2H). ¹³C{¹H} NMR (100 MHz, CDCl₃): δ δ 21.67, 32.96, 43.65, 131.47, 135.79, 169.19.

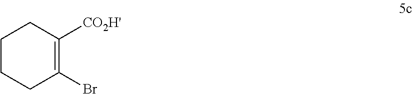

Obtained as white crystalline solid (84% yield). 1H NMR (400 MHz, CDCl₃): δ 1.72 (m, 4H), 2.43 (m, 2H), 2.64 ppm (s, 2H). ¹³C{¹H} NMR (100 MHz, CDCl₃): δ 21.46, 23.99, 28.76, 38.13, 129.51, 129.79, 172.72.

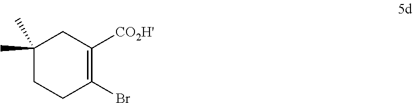

Obtained as white crystalline solid (81% yield). ¹H NMR (400 MHz, CDCl₃): δ 0.97 (s, 6H), 1.48 (t, 2H), 2.22 (s, 2H), 2.65 ppm (tt, 2H). ¹³C{¹H} NMR (100 MHz, CDCl₃): δ 27.68, 28.71, 36.10, 36.57, 42.13, 128.38, 128.76, 172.54. HRMS (ESI, m/z) for C₉H₁₄BrO₂ [M+H]⁺: calcd, 233.0172; found, 233.0169.

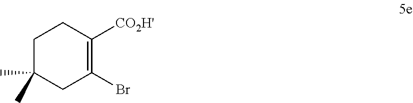

Obtained as white crystalline solid (87% yield). ¹H NMR (400 MHz, CDCl₃): δ 0.98 (s, 6H), 1.48 (t, 2H), 2.42-2.50 ppm (m, 4H). ¹³C{¹H} NMR (100 MHz, CDCl₃): δ 26.50, 27.77, 31.77, 34.15, 51.73, 128.04, 129.24, 172.58. HRMS (ESI, m/z) for C₉H₁₄BrO₂ [M+H]⁺: calcd, 234.0205; found, 234.0203.

### General Procedure for the Synthesis of the Beta-Ketoamide

Mixture A: 1,1′-Carbonyldiimidazole (1.1 equiv.) was added in 3 portions to a solution of the carboxylic acid (1.0 equiv.) and THF (0.2 M). The resulting mixture was stirred for 1 hour at room temperature.
Mixture B: To solution of LHMDS (3.2 equiv., 1.0 M in THF) and THF (0.5 M) at −78° C., N-methoxy-N-methylacetamide (3.2 eq) was added dropwise using a syringe pump. The mixture was stirred for 1 hr at the same temperature.
Mixture A was transferred slowly to Mixture B using a cannula. The creamy mixture was warmed to ambient temperature and stirred for 12-15 hrs. After the reaction, the mixture was quenched with saturated NH₄Cl, stirred for an additional 15 mins and extracted with ethyl acetate. The organic layer pool was washed with brine, dried over Na₂SO₄, and concentrated under reduced pressure. The crude product was purified by automatic flash column chromatography on silica gel (gradient elution from 5% to 40% EtOAc-hexanes).

Obtained as light orange crystalline solid (91% yield, 1:1 mixture of keto and enol tautomers).

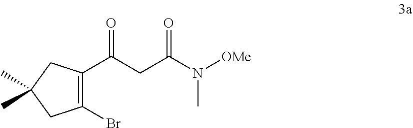

¹H NMR (400 MHz, CDCl₃): δ 1.14 (s, 6H), 1.15 (s, 6H), 2.44 (t, 2H), 2.50 (t, 2H), 2.64 (t, 2H), 2.71 (t, 2H), 3.23 (s, 6H), 3.69 (s, 3H), 3.73 (s, 3H), 4.04 (s, 2H), 5.93 (s, 1H), 13.81 ppm (s, 1H). ¹³C{¹H} NMR (100 MHz, CDCl₃): δ 29.29, 29.53, 31.94 (br), 32.15 (br), 36.77, 36.86, 47.03, 47.82, 47.88, 57.69, 58.24, 61.44, 61.61, 88.16, 122.69, 129.56, 133.27, 138.98, 167.68, 168.65, 172.50, 191.69. HRMS (ESI, m/z) for C₁₂H₁₉BrNO₃ [M+H]⁺: calcd, 305.0576; found, 305.0571.

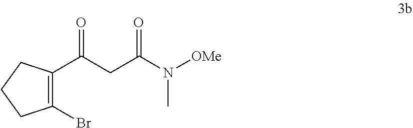

Obtained as light pink crystalline solid (90% yield, 1:1 mixture of keto and enol tautomers). ¹H NMR (400 MHz, CDCl₃): δ 1.96 (sextet, 4H), 2.64 (tt, 2H), 2.70 (tt, 2H), 2.83 (tt, 2H), 2.91 (tt, 2H), 3.23 (s, 6H), 3.69 (s, 3H), 3.73 (s, 3H), 4.05 (s, 2H), 5.96 (s, 1H), 13.81 ppm (s, 1H). ¹³C{¹H} NMR (100 MHz, CDCl₃): δ 21.26, 21.55, 31.96 (br), 32.16 (br), 33.28, 33.42, 43.43, 44.22, 47.09, 61.46, 61.62, 88.29, 124.54, 131.41, 134.48, 140.05, 167.70, 168.69, 172.50, 191.70. HRMS (ESI, m/z) for C₁₀H₁₅BrNO₃ [M+H]⁺: calcd, 277.0263; found, 277.0260.

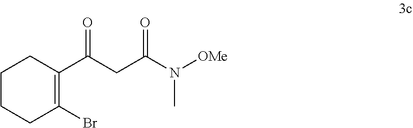 [SMILES: CON(C)C(=O)CC(=O)C1=C(Br)CCCC1]

Obtained as light yellow solid (81% yield, 1:1 mixture of keto and enol tautomers). ¹H NMR (400 MHz, CDCl₃): δ 1.66-1.78 (m, 8H), 2.32-2.40 (m, 4H), 2.54-2.62 (m, 4H), 3.22 (s, 3H), 3.23 (s, 3H), 3.71 (s, 3H), 3.72 (s, 3H), 3.98 (s, 2H), 5.64 (s, 1H), 13.78 ppm (s, 1H). ¹³C{¹H} NMR (100 MHz, CDCl₃): δ 21.38, 21.77, 24.14, 24.30, 29.02, 29.76, 31.80 (br), 32.12 (br), 36.73, 37.01, 47.01, 61.54, 61.84, 89.23, 122.37, 123.00, 133.45, 138.91, 167.95, 172.33, 173.35, 199.27. HRMS (ESI, m/z) for C₁₁H₁₇BrNO₃ [M+H]⁺: calcd, 291.0420; found, 291.0415.

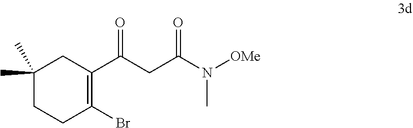

Obtained as light orange crystalline solid (79% yield, 3:2 mixture of keto and enol tautomers). ¹H NMR (400 MHz, CDCl₃): δ 0.98 (s, 12H), 1.49 (t, 4H), 2.15 (m, 4H), 2.58 (m, 4H), 3.22 (s, 3H), 3.23 (s, 3H), 3.71 (s, 3H), 3.73 (s, 3H), 3.98 (s, 2H), 5.63 (s, 1H), 13.77 ppm (s, 1H). ¹³C{(¹H} NMR (100 MHz, CDCl₃): δ 27.60, 27.72, 28.66, 28.88, 31.83 (br), 32.14 (br), 34.74, 34.97, 36.77, 36.87, 42.36, 43.17, 46.97, 61.53, 61.85, 89.26, 121.17, 121.77, 132.35, 137.94, 167.94, 172.34, 173.40, 199.21. HRMS (ESI, m/z) for C₁₃H₂₁BrNO₃ [M+H]⁺: calcd, 318.0699; found, 318.0690.

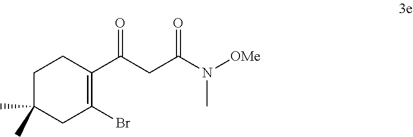

Obtained as light orange crystalline solid (80% yield, 3:2 mixture of keto and enol tautomers). ¹H NMR (400 MHz, CDCl₃): δ 0.99 (s, 12H), 1.46 (m, 4H), 2.38 (m, 8H), 3.21 (s, 3H), 3.23 (s, 3H), 3.71 (s, 3H), 3.72 (s, 3H), 3.98 (s, 2H), 5.65 (s, 1H), 13.80 ppm (s, 1H). ¹³C{H} NMR (100 MHz, CDCl₃): δ 26.86, 27.35, 27.70, 27.81, 31.77 (br), 31.81, 31.91, 32.06 (br), 34.03, 34.37, 47.06, 50.28, 50.55, 61.45, 61.76, 89.20, 121.41, 121.87, 132.02, 137.49, 167.84, 172.26, 173.14, 198.93. HRMS (ESI, m/z) for C₁₃H₂₁BrNO₃ [M+H]⁺: calcd, 319.0733; found, 319.0731.

### General Procedure for the Cu-Catalyzed Benzannulation Reaction

A mixture of the ketoamide (3a-3e) (1.0 equiv.), Cs₂CO₃ (2.0 equiv.) and DMF (0.25 M) was stirred at ambient temperature for 5 mins. 3-(tert-butoxy)-3-oxopropanoic acid-(8) was added (1.5 equiv.) to the suspension followed by copper (I) bromide (0.1 equiv.) after 15 mins. The green mixture was heated to 60° C. and stirred for 24 hrs. The mixture was then cooled to room temperature, diluted with distilled water and extracted with ethyl acetate. The organic layer pool was washed with brine, dried over Na₂SO₄, and concentrated under reduced pressure. The crude product was purified by automatic flash column chromatography on silica gel (gradient elution from 10% to 80% EtOAc-hexanes).

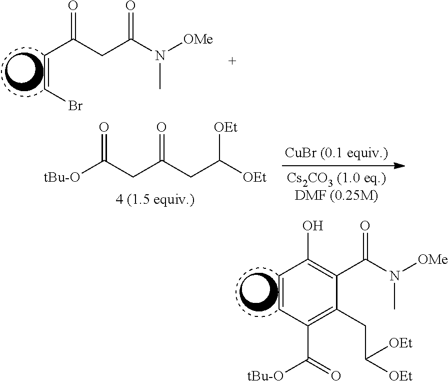

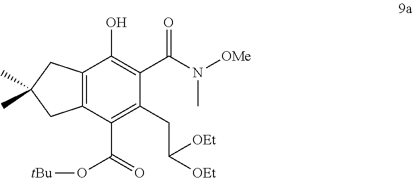

Obtained as light orange solid (79% yield). ¹H NMR (400 MHz, CDCl₃): δ 1.06-1.17 (m, 12H), 1.58 (s, 9H), 2.66 (s, 2H), 2.82 (q, 2H), 3.00-3.22 (m, 2H), 3.28 (s, 3H), 3.32-3.48 (m, 2H), 3.50-3.68 (m, 5H), 4.55 (t, 1H), 7.23 ppm (br s, 1H). ¹³C{¹H} NMR (100 MHz, CDCl₃): δ 15.32, 15.39, 28.37, 29.06, 29.09, 35.73, 39.71, 43.55, 48.80, 61.16 (br), 62.00 (br), 62.68, 81.31, 103.86, 121.76, 124.92, 128.87, 133.39, 146.51, 151.05, 168.02. HRMS (ESI, m/z) for C₂₅H₃₈NO₇ [M−H]⁻: calcd, 464.2654; found, 464.2655.

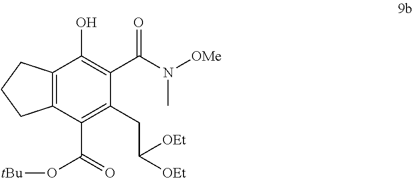 [SMILES: CCOC(Cc1c(C(=O)N(C)OC)c(O)c2c(c1C(=O)OC(C)(C)C)CCC2)OCC]

Obtained as light-yellow solid (79% yield). ¹H NMR (400 MHz, CDCl₃): δ 1.09 (t, 3H), 1.14 (t, 3H), 1.58 (s, 9H), 1.94-2.09 (m, 2H), 2.78 (t, 2H), 2.96 (sextet, 2H), 3.04-3.23 (m, 2H), 3.27 (br s, 3H), 3.32-3.52 (m, 2H), 3.52-3.61 (m, 2H), 3.65 (br s, 3H), 4.56 (t, 1H), 7.54 ppm (br s, 1H). ¹³C{¹H} NMR (100 MHz, CDCl₃): δ 15.33, 15.42, 24.54, 28.36, 29.00, 33.94, 35.70, 61.25 (br), 61.84 (br), 62.62, 81.33, 103.76, 121.87, 124.69, 130.06, 133.25, 147.10, 150.76, 168.06. HRMS (ESI, m/z) for C₂₃H₃₄NO₇ [M−H]⁻: calcd, 436.2341; found, 436.2343.

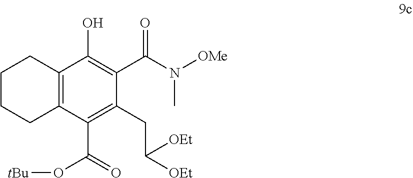 [SMILES: CCOC(Cc1c(C(=O)N(C)OC)c(O)c2c(c1C(=O)OC(C)(C)C)CCCC2)OCC]

Obtained as light yellow crystalline solid (76% yield). ¹H NMR (400 MHz, CDCl₃): δ δ 1.11 (t, 3H), 1.15 (t, 3H), 1.59 (s, 9H), 1.74 (m, 4H), 2.59 (m, 2H), 2.69 (m, 2H), 2.89-3.12 (m, 2H), 3.28 (s, 3H), 3.33-3.67 (m, 7H), 4.58 (t, 1H), 7.84 ppm (s, 1H). ¹³C{¹H} NMR (100 MHz, CDCl₃): δ 15.25, 15.34, 20.80, 22.01, 22.46, 23.12, 27.27, 28.17, 36.43, 61.33 (br), 61.58, 62.72 (br), 81.86, 103.61, 118.85, 124.29, 129.19, 129.26, 136.71, 152.53, 169.18. HRMS (ESI, m/z) for C₂₄H₃₆NO₇ [M−H]⁻: calcd, 450.2497; found, 450.2502.

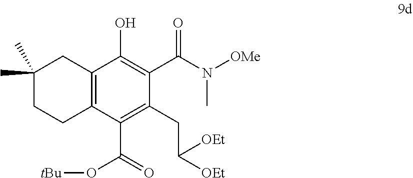

Obtained as light yellow solid (75% yield). ¹H NMR (400 MHz, CDCl₃): δ δ 0.99 (s, 6H), 1.10 (t, 3H), 1.15 (t, 3H), 1.53 (t, 2H), 1.60 (s, 9H), 2.44 (m, 2H), 2.74 (t, 2H), 2.89-3.10 (m, 2H), 3.29 (s, 3H), 3.33-3.64 (m, 7H), 4.57 (t, 1H), 7.74 ppm (s, 1H). ¹³C{¹H} NMR (100 MHz, CDCl₃): δ δ 15.24, 15.34, 24.32, 27.99, 28.17, 28.34, 28.43, 35.07, 36.68, 36.76, 61.37 (br), 61.84, 62.87 (br), 81.95, 103.75, 118.35, 123.43, 128.98, 129.23, 135.61, 153.03, 169.29. HRMS (ESI, m/z) for C₂₆H₄₀NO₇ [M−H]⁻: calcd, 478.2810; found, 478.2815.

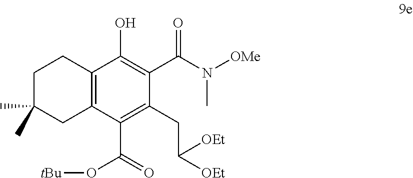

Obtained as light yellow solid (80% yield). ¹H NMR (400 MHz, CDCl₃): δ 0.96 (s, 6H), 1.10 (t, 3H), 1.14 (t, 3H), 1.55 (t, 2H), 1.59 (s, 9H), 2.46 (s, 2H), 2.67 (t, 2H), 2.90-3.12 (m, 2H), 3.30 (s, 3H), 3.33-3.64 (m, 7H), 4.56 (t, 1H), 7.64 ppm (s, 1H). ¹³C{¹H} NMR (100 MHz, CDCl₃): δ 15.29, 15.38, 20.80, 28.08, 28.15, 28.22, 28.90, 34.53, 36.71, 41.05, 61.48 (br), 61.94, 62.88 (br), 81.94, 103.79, 118.21, 122.71, 129.52, 129.60, 136.36, 152.77, 169.25. HRMS (ESI, m/z) for C₂₆H₄₀NO₇ [M−H]⁻: calcd, 478.2810; found, 478.2812.

### General Procedure for the Partial Reduction of the Weinreb Amide

To a solution of the Weinreb amide (9a-9e) (1.0 equiv.) in THF (0.1 M) cooled to 0° C., lithium aluminum hydride (1.0 equiv., 1.0 M in THF) was added dropwise. The mixture was stirred for 1 hour at 0° C. While the mixture is still cold, ethyl acetate was added slowly followed by 1M HCl. The layers were separated, and the aqueous layer was extracted with ethyl acetate. The organic layer pool was washed with brine, dried over Na₂SO₄, and concentrated under reduced pressure. The crude product was purified by automatic flash column chromatography on silica gel (gradient elution from 5% to 40% EtOAc-hexanes).

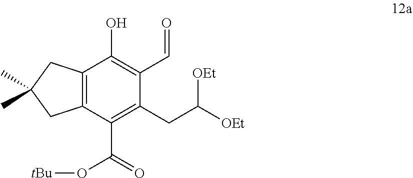

Obtained as white crystalline solid (46% yield). ¹H NMR (400 MHz, CDCl₃): δ 1.14 (t, 6H), 1.17 (s, 6H), 1.60 (s, 9H), 2.72 (s, 2H), 2.82 (s, 2H), 3.32 (d, 2H), 3.41 (dq, 2H), 3.69 (dq, 2H), 4.06 (t, 1H), 10.35 (s, 1H), 12.54 ppm (s, 1H). ¹³C{¹H} NMR (100 MHz, CDCl₃): δ 15.32, 28.34, 29.14, 33.44, 39.64, 43.18, 49.08, 63.60, 82.19, 104.00, 117.78, 124.72, 130.15, 138.41, 152.07, 160.51, 167.66, 197.61. HRMS (ESI, m/z) for C₂₃H₃₃O₆ [M−H]−: calcd, 405.2283; found, 405.2286.

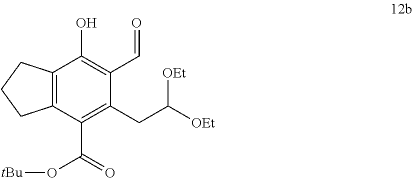 [SMILES: CCOC(Cc1c(C=O)c(O)c2c(c1C(=O)OC(C)(C)C)CCC2)OCC]

Obtained as white crystalline solid (55% yield). ¹H NMR (400 MHz, CDCl₃): δ 1.14 (t, 6H), 1.60 (s, 9H), 2.11 (quint, 2H), 2.91 (t, 2H), 3.03 (t, 2H), 3.32 (d, 2H), 3.41 (dq, 2H), 3.70 (dq, 2H), 4.66 (t, 1H), 10.36 (s, 1H), 12.58 ppm (s, 1H). ¹³C{¹H} NMR (100 MHz, CDCl₃): δ 15.31, 24.31, 28.32, 28.68, 33.49, 34.39, 63.59, 82.18, 104.01, 117.72, 124.48, 131.04, 138.32, 152.77, 160.31, 167.74, 197.61. HRMS (ESI, m/z) for C₂₁H₃₁O₆[M+H]⁺: calcd, 379.2115, found, 379.2111.

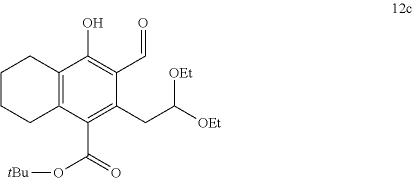 [SMILES: CCOC(Cc1c(C=O)c(O)c2c(c1C(=O)OC(C)(C)C)CCCC2)OCC]

Obtained as white crystalline solid (43% yield). ¹H NMR (400 MHz, CDCl₃): δ 1.13 (t, 6H), 1.61 (s, 9H), 1.78 (m, 4H), 2.69 (m, 4H), 3.15 (d, 2H), 3.39 (dq, 2H), 3.69 (dq, 2H), 4.61 (t, 1H), 10.31 (s, 1H), 12.74 ppm (s, 1H). ¹³C{¹H} NMR (100 MHz, CDCl₃): δ 15.32, 21.73, 22.26, 22.45, 27.82, 28.21, 33.92, 63.58, 82.60, 103.92, 115.77, 125.57, 128.86, 133.42, 142.82, 161.64, 168.94, 197.25. HRMS (ESI, m/z) for C₂₂H₃₃O₆ [M+H]⁺: calcd, 393.2272; found, 393.2457.

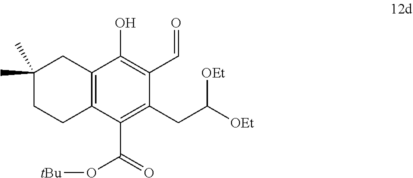

Obtained as white crystalline solid (53% yield). ¹H NMR (400 MHz, CDCl₃): δ 1.00 (s, 6H), 1.13 (t, 6H), 1.56 (t, 2H), 1.62 (s, 9H), 2.46 (s, 2H), 2.75 (t, 2H), 3.16 (d, 2H), 3.39 (dq, 2H), 3.69 (dq, 2H), 4.62 (t, 1H), 10.32 (s, 1H), 12.77 ppm (s, 1H). ¹³C{¹H} NMR (100 MHz, CDCl₃): δ 15.32, 25.05, 28.23, 28.26, 28.29, 33.96, 34.86, 35.95, 63.57, 82.64, 103.89, 115.88, 124.93, 128.66, 133.38, 141.68, 161.91, 168.97, 197.25. HRMS (ESI, m/z) for C₂₄H₃₇O₆ [M+H]⁺: calcd, 421.2585; found, 421.2575.

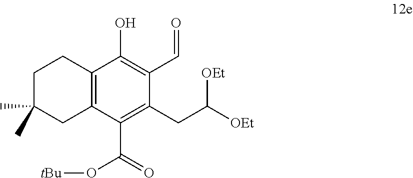

Obtained as white crystalline solid (39% yield). ¹H NMR (400 MHz, CDCl₃): δ 0.98 (s, 6H), 1.13 (t, 6H), 1.57 (t, 2H), 1.61 (s, 9H), 2.47 (s, 2H), 2.70 (t, 2H), 3.16 (d, 2H), 3.39 (dq, 2H), 3.69 (dq, 2H), 4.61 (t, 1H), 10.32 (s, 1H), 12.75 ppm (s, 1H). ¹³C{¹H} NMR (100 MHz, CDCl₃): δ 15.31, 20.12, 28.12, 28.23, 28.96, 33.95, 34.29, 41.60, 63.58, 82.56, 103.89, 115.80, 124.28, 129.10, 133.67, 142.27, 161.52, 168.89, 197.29. HRMS (ESI, m/z) for C₂₄H₃₅O₆ [M−H]⁻: calcd, 419.2439, found, 419.2444.

### General Procedure for the Lactonization to Form Illudalic Acid (IA1) and Analogues

To a solution of the aldehyde (9a-9e) in acetone (0.04 M) stirred at room temperature was added 6M HCl (150 equiv.), and the solution was allowed to stir for 5 hours (can also let go overnight). The clear, light-yellow solution would slowly become cloudy and ultimately a dense precipitate would form signifying the end of the reaction. The solid was isolated via vacuum filtration, washed with water, cold ether, and dried. The filter cake was removed by washing it through the filter into a evaporation flask with acetone, the pure product was recovered in vacuo.

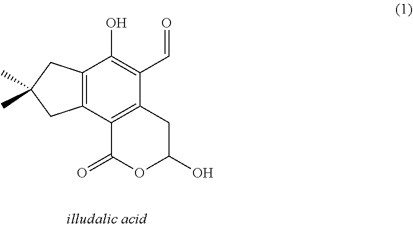

Obtained as white powdery solid (81% yield) (MP=201-203° C.). ¹H NMR (600 MHz, DMSO-d₆): δ 1.12 (s, 3H), 1.13 (s, 3H), 2.64 (s, 2H), 3.09 (s, 2H), 3.38 (dd, 1H), 3.55 (dd, 1H), 5.74 (dt, 1H), 7.71 (d, 1H), 10.32 (s, 1H), 12.02 ppm (s, 1H). ¹³C{H} NMR (150 MHz, DMSO-d₆): δ 28.74, 30.45, 39.06, 42.49, 49.83, 94.48, 114.01, 116.71, 130.00, 142.14, 155.86, 161.48, 162.72, 195.31.

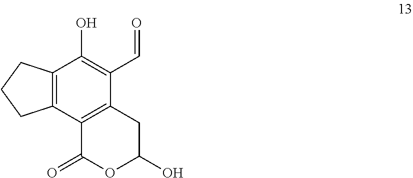 [SMILES: O=Cc1c(O)c2c(c3c1CC(O)OC3=O)CCC2]

Obtained as white powdery solid (80% yield) (MP=206-209° C.). ¹H NMR (600 MHz, DMSO-d₆): δ 2.05 (pentet, 2H), 2.81 (t, 2H), 3.24 (t, 2H), 3.38 (dd, 1H), 3.54 (dd, 1H), 5.71-5.77 (m, 1H), 7.70 (d, 1H), 10.32 (s, 1H), 12.08 ppm (s, 1H). ¹³C{H} NMR (150 MHz, DMSO-d₆): δ 23.76, 28.03, 30.44, 35.18, 94.46, 113.80, 116.59, 131.03, 142.16, 156.83, 161.31, 162.70, 195.36. HRMS (ESI, m/z) for C₁₃H₁₃O₅ [M+H]⁺: calcd, 249.0757; found, 249.0755.

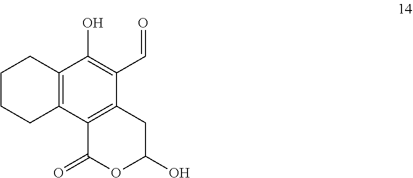 [SMILES: O=Cc1c(O)c2c(c3c1CC(O)OC3=O)CCCC2]

Obtained as white powdery solid (84% yield) (MP=177-179° C.). ¹H NMR (600 MHz, DMSO-d₆): δ 1.61-1.75 (m, 4H), 2.54-2.67 (m, 2H), 3.04-3.12 (m, 2H), 3.31 (dd, 1H), 3.57 (dd, 1H), 5.66 (dt, 1H), 7.67 (d, 1H), 10.27 (s, 1H), 12.77 ppm (s, 1H). ¹³C{¹H} NMR (150 MHz, DMSO-d₆: δ 20.81, 21.93, 22.38, 29.49, 30.46, 93.97, 114.11, 116.03, 125.45, 140.93, 149.80, 162.34, 163.08, 196.48. HRMS (ESI, m/z) for C₁₄H₁₃O₅ [M−H]⁻: calcd, 261.0768, found, 261.0771.

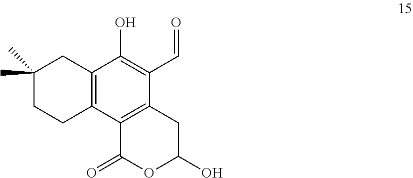

Obtained as white powdery solid (87% yield) (MP=196-197° C.). ¹H NMR (600 MHz, DMSO-d₆): δ 0.92 (s, 3H), 0.93 (s, 3H), 1.40-1.50 (m, 2H), 2.36 (q, 2H), 3.10 (qt, 2H), 3.29 (dd, 1H), 3.55 (dd, 1H), 5.64 (dt, 1H), 7.65 (d, 1H), 10.24 (s, 1H), 12.77 ppm (s, 1H). ¹³C{¹H} NMR (150 MHz, DMSO-d₆): δ 26.70, 27.17, 27.57, 27.94, 30.42, 34.42, 35.93, 93.94, 114.28, 115.75, 124.76, 141.08, 148.54, 162.42, 163.32, 196.522. HRMS (ESI, m/z) for C₁₆H₁₉O₅ [M+H]⁺: calcd, 291.1227; found, 291.1221.

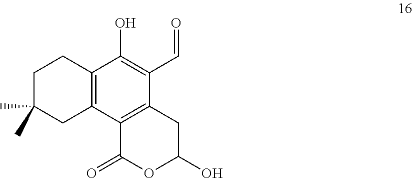

Obtained as white powdery solid (85% yield (MP=185-187° C.). ¹H NMR (600 MHz, DMSO-d₆): δ 0.90 (s, 3H), 0.94 (s, 3H), 1.51 (t, 2H), 2.56-2.68 (m, 2H), 2.88 (q, 2H), 3.29 (dd, 1H), 3.59 (dd, 1H), 5.66 (dt, 1H), 7.67 (d, 1H), 10.27 (s, 1H), 12.77 ppm (s, 1H). ¹³C{¹H} NMR (150 MHz, DMSO-d₆): δ 19.96, 27.38, 28.23, 28.34, 30.52, 33.05, 42.80, 93.96, 114.10, 116.20, 124.20, 141.19, 148.82, 162.35, 162.96, 196.46. HRMS (ESI, m/z) for C₁₆H₁₇O₅ [M−H]⁻: calcd, 289.1081; found, 289.1084.

### General Procedure for the One-Pot Reduction-Lactonization

To a mixture of the Weinreb amide (9a-or-9b) (1.0 equiv.) in THF (0.1 M) cooled at 0° C., lithium aluminum hydride (1.0 equiv., 1.0 M in THF) was added dropwise. The mixture was stirred for 1 hour at 0° C. While the mixture is still cold, 6M HCl (150 equiv.) was added dropwise. The mixture was warmed to ambient temperature and stirred for 2 hrs. After the reaction, the mixture was extracted with ethyl acetate. The organic layer pool was washed with brine, dried over Na₂SO₄, and concentrated under reduced pressure. The crude product was purified by automatic flash column chromatography on silica gel (gradient elution from 20% to 100% EtOAc-hexanes).

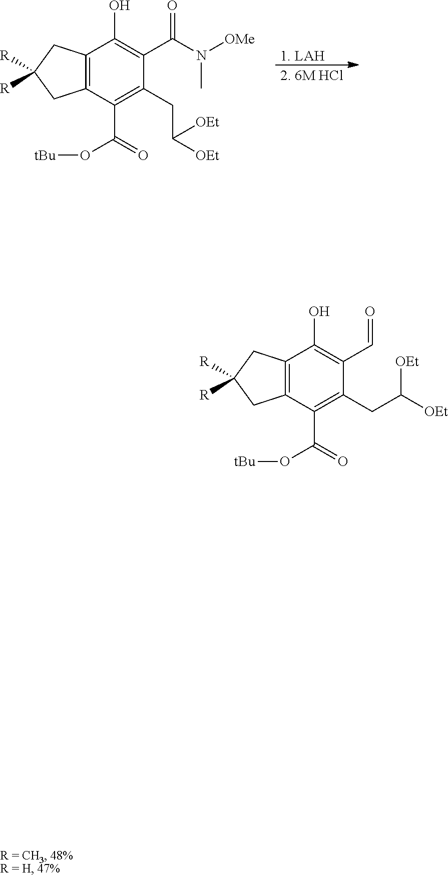

Alternate Pharmacophores (Compounds) of this Invention

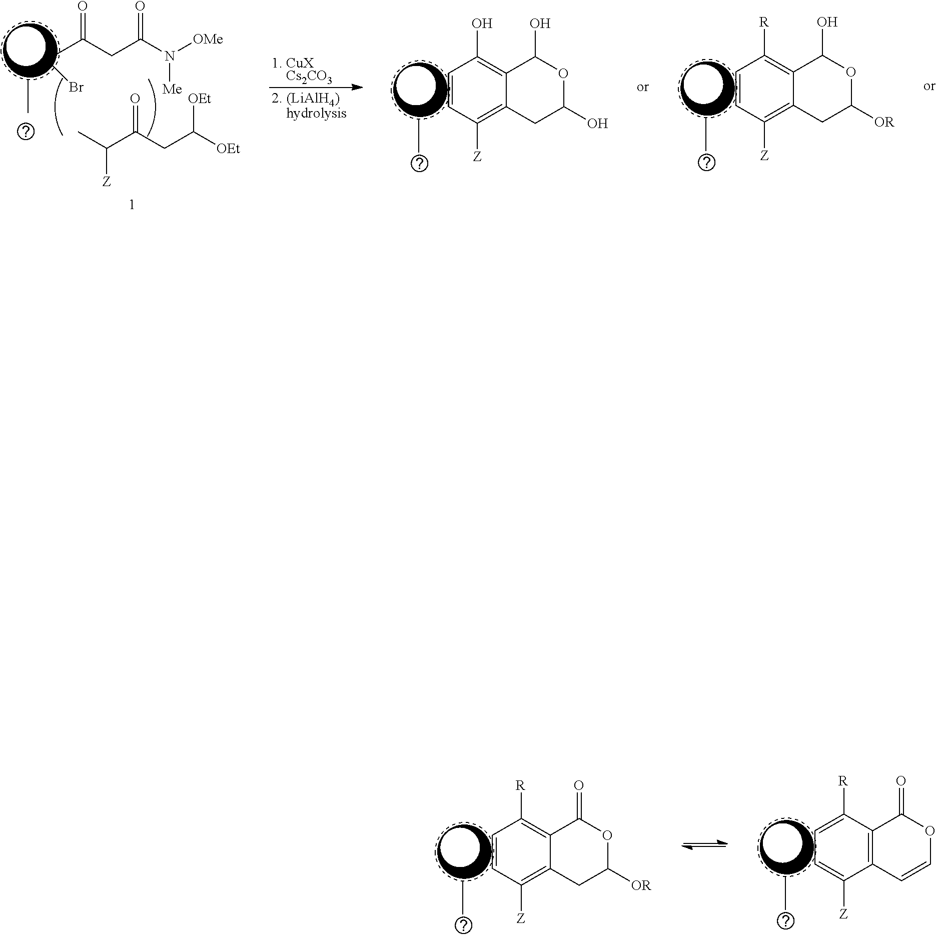

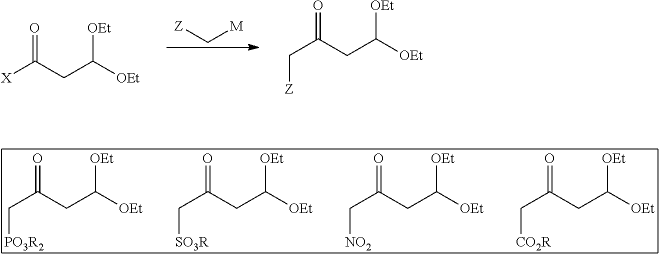

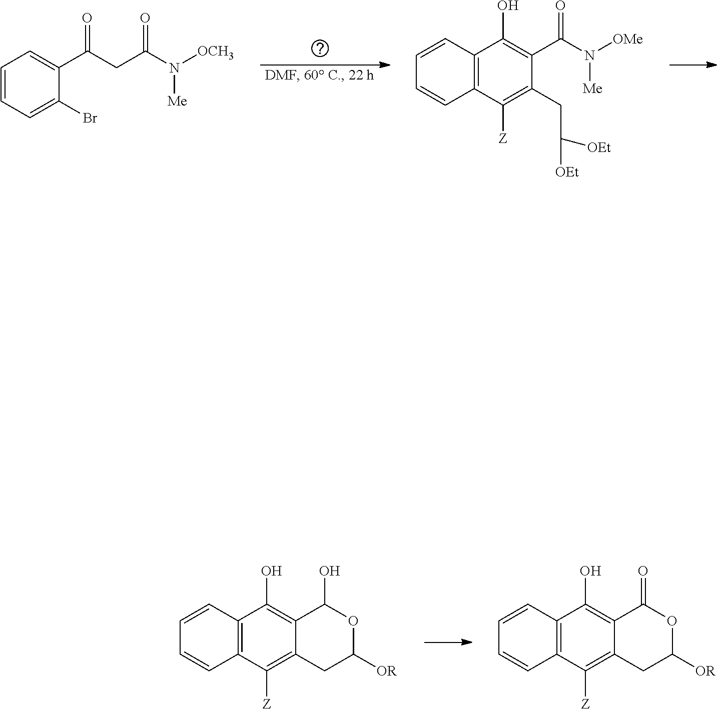

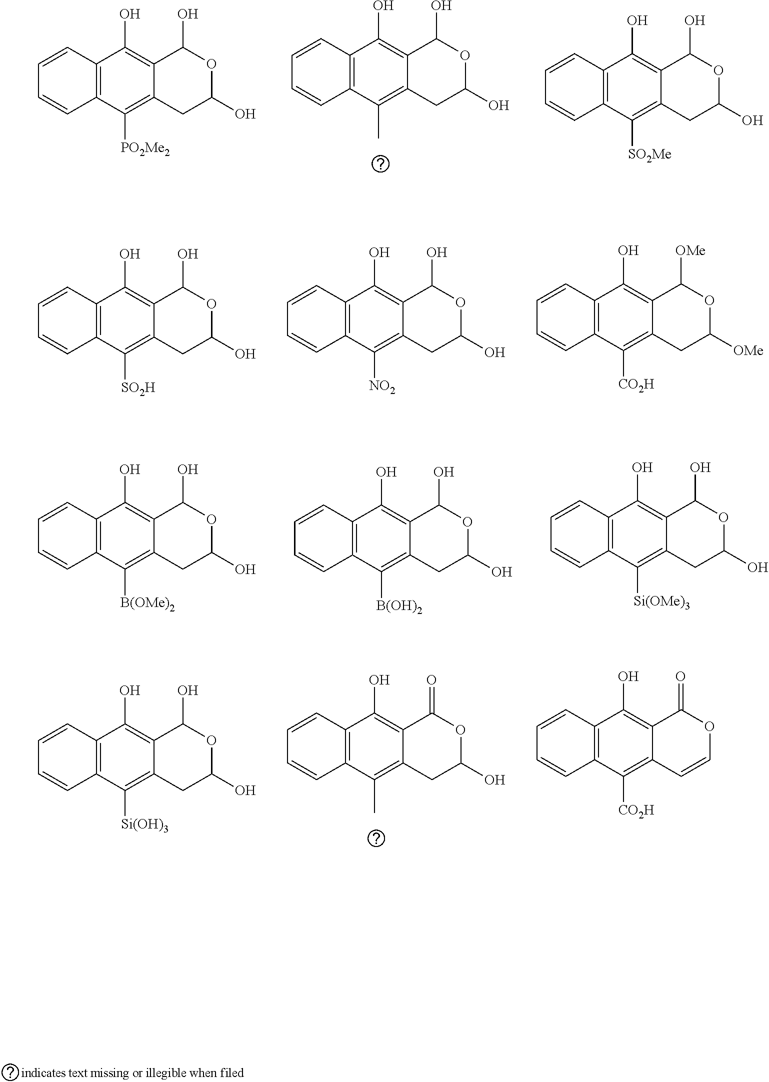

Prophetically, we prepare illudalogs with alternative pharmacophores based on the hypothesis that these alternative pharmacophores will be potent by virtue of favorable molecular recognition and rapid covalent ligation of the enzymes of interest. We replace the carboxylic acid/carboxylate group of the established trifunctional pharmacophore of illudalic acid with phosphonate, sulfonate, and nitro groups, for example, which will generally be prepared as outlined in the Synthesis Scheme 5 above by analogy to the current synthesis of illudalogs. We will also target boronate and silicate groups and alternative oxidation states of the aldehydes by what we anticipate will routine synthetic organic protecting group and functional group manipulations. These groups as alternatives to the carboxylic acid are not expected to engage with the neighboring aldehyde, such that the pharmacophore dialdehyde (masked as the dialdehyde hydrate, as shown), will be more readily available to engage with active site cysteine and/or other protein residues for covalent ligation. The alternative pharmacophores are incorporated into the IA3 (naphthalene) series above for example; they will be incorporated similarly into other core scaffolds (IA1-IAn) as indicated by the generic structures at the top. Compounds incorporating alternative pharmacophores are logical extensions of the current invention and shall create new opportunities for therapeutic development.

Alternative functional groups can similarly engage with conserved arginine and cysteine active site residues in ways that alter K₁ and k_{inact}, and thereby alter potency. We made acrylate IA3-5A and coumarin IA3-56C to probe the effect of Michael acceptors in lieu of the aldehyde at position 5 (FIG. 20, middle top). We replace the carboxylic acid with a nitro group and sulfonic and phosphonic acid groups (IA3-1Z). Phosphonic acid (Z═PO₃H₂) mimics native phosphotyrosine substrates. Like the carboxylic acid, these groups are expected to engage in hydrogen bonding with Arg1559 to varying degrees. Unlike the carboxylic acid, these groups are not expected to engage the proximal aldehyde covalently, leaving the dialdehyde hydrate more available to react with Cys1553. Oxidation of IA3-4Z (or IA3 itself) to isocoumarin IA3-1Z5[O] creates an alternative electrophilic presentation for reaction with Cys1553. (IA3-1Z5[O] could emerge as a hydroxy-lactone, like IA3.) We expect that these structural changes to the inhibitor will significantly change Kᵢ and k_{inact} values in ways that provide important insights into the binding mechanism. Modifications that result in stronger and/or more potent inhibitors will be incorporated into future designs.

We prepare IA3-1Z from variants of β-keto ester 2 (cf. FIG. 4—bottom, and FIG. 1), replacing the ester with the appropriate Z group or functional equivalents. For example, phosphonate 2p, by analogy to β-keto ester 2, is proposed to undergo benzannulation with the various β-keto amides 4 that are already in inventory (FIG. 20 bottom). Amide reduction and hydrolysis is expected to provide a library of phosphono-illudalogs (IAn-1P). Alternatively, if we omit reduction and leave the 5-position at the carboxylic acid oxidation level, then hydrolysis produces IAn-1P-5[O], which will present as an alternative hydroxy-lactone or isocoumarin. Similar processes are envisioned for nitro- and sulfono-illudalogs.

Knockout Illudalog Variants of this Invention

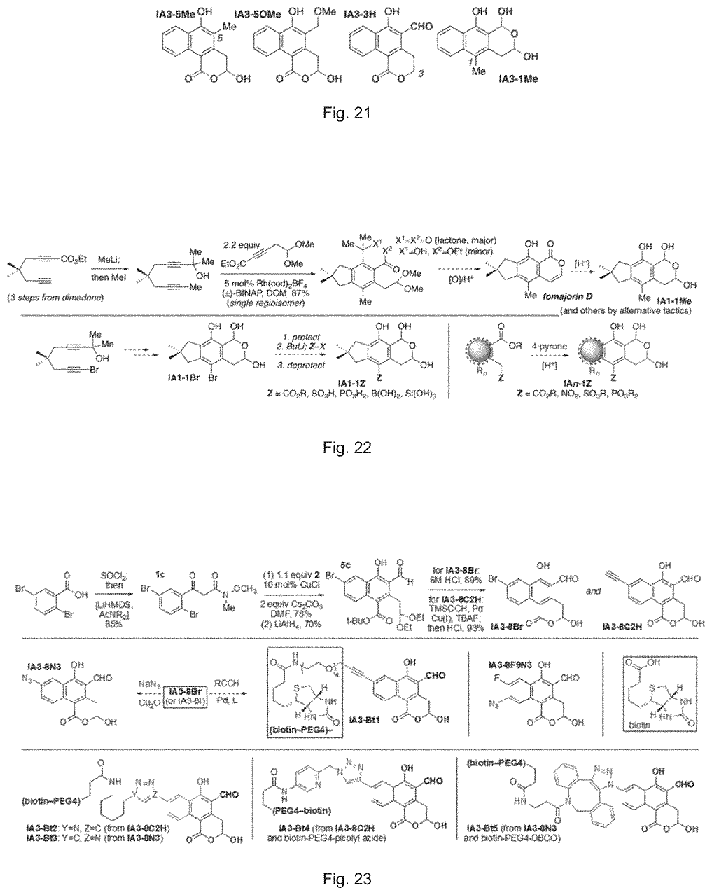

The key trifunctional pharmacophore is necessary for activity. Each of the three functional groups is postulated to play a role in binding, but systematic studies into these postulates require methodology for manipulating each functional group independent of the others. The objective here is to achieve selective reduction of each functional group. By-product alcohol 7b discussed above (cf. FIG. 15, top) is one such knockout, which will be further reduced or methylated to en route to IA3-5Me or IA3-5OMe (FIG. 21). Sodium borohydride reduction of IA3, lactone formation, and reoxidation is expected to provide IA3-3H. Finally, phenol protection of 4b (cf. FIG. 15, top) will block the phenol-directed reduction of the Weinreb amide, changing reduction chemoselectivity to favor the ester. Exhaustive reduction of the ester to the methyl group, then partial reduction of the amide and deprotections of the phenol and aldehyde will provide IA3-1Me.

Based on the postulated two-stage PTPRD binding mechanism, we expect IA3-3H to be inactive in vitro because it cannot form the initial salt bridge with the arginine residue or the subsequent covalent ligation to the cysteine residue. IA3-5Me or IA3-5OMe can form the salt bridge but not the covalent adduct, so we expect them to be weak, reversible inhibitors. (Reversible inhibitors might be useful for some future PROTAC applications.) IA3-1Me can form the covalent adduct and therefore could be an irreversible inhibitor, but it will likely have little to no potency because it cannot form the initial salt bridge.

Initial Inhibitor Screen Assays

We screen illudalogs against a panel of PTPs including PTPRD, as illustrated in FIG. 16. Enzyme activity assays are performed in black 96-well plates containing a total volume of 100 μL in each well. Typical experimental conditions are 5-10 nM enzyme, 1-10 μM DiFMUP, and 250 nM inhibitor at 37° C. Prior to each assay, the enzyme is activated by incubating in HEPES buffer (50 mM HEPES pH 7.5, 100 mM NaCl, 0.02% tween, 1 mM EDTA) with 5 μL of 100 mM DTT on ice for 30 min. Buffer, enzyme, and either a DMSO control or the compound of interest are added to each well of the 96-well plate and incubated for 30 min at 37° C., at which point the enzyme substrate DiFMUP is added to initiate the reaction. The fluorescence resulting from the hydrolysis of DiFMUP is measured every 60 s for 30 min (minutes) in a SpectraMax M5 plate reader with excitation and emission wavelengths of 350 and 455 nm, respectively.

IC₅₀ Curve and Time-Dependent Inhibition Assays

Assays to determine the IC₅₀ values for each hit compound are carried out as described in the Initial inhibitor screen assays section (above) using varying concentrations of inhibitor (generally 0.1 nM-10 μM). Assays to determine k_{b}, values are likewise conducted as described above using varying concentrations of inhibitor (e.g., 0.1 nM-10 μM) and varying incubation times (5-120 min). Buffer and enzyme are initially added to each well, followed by sequential addition of inhibitor starting from the longest incubation time to the shortest. Once all inhibitor has been preincubated for the desired time, assays are initiated by addition of DiFMUP to each well and the increase in fluorescence is monitored as above. The k_{obs} values are obtained as described previously and plotted against inhibitor concentrations to obtain Kᵢ and k_{inact} values.^{15,69} Stopped flow kinetics instrumentation is available at UofU for use with these experiments as needed.

We prepare and maintain a supply of >100 illudalogs with diverse core structures, substitution patterns, and trifunctional pharmacophores (and knockout variants). These illudalog compounds of this invention support broader pharmacology efforts aimed at identifying potent and selective PTPRD inhibitors. We provide herein a comprehensive library to support a detailed examination of illudalog SAR as PTPRD inhibitors.

As an alternative to IA3 knockout variants, we can access alternative pharmacophores and knockout variants of IA1 by analogy. We develop a synthesis of fomajorin D connected to a broader interest in the illudalane sesquiterpenes. Phenol-directed reduction of fomajorin D would provide IA1-1Me, for example (FIG. 22, top). An analogous synthesis of IA1-1Br (FIG. 22, bottom left) would enable production of diverse IA1-1Z illudalogs, including boronic acid and silanol variants. Finally, the alternative 1-step annulation of IA3 may be adapted to illudalic acid and other illudalogs, for example by starting from phosphono- or sulfono-benzoates or acrylates (FIG. 22, bottom right). Horner-Wadsworth-Emmons-type pathways could complicate reactions of phosphono-acrylates, but that would not preclude broader exploration of this alternative solution.

### Functional Probes

We validate the synthesis of illudalog-based functional probes for bioimaging, proteomics, etc. to support future PTPRD cell biology. We develop click chemistry of bromo-, azido-, and/or ethynyl-IA3 derivatives suitable for attaching biotin, fluorescent labels, and other functional tags under various conditions, including in vivo. Specifically, we first prepare a variety of biotin-tagged illudalogs to ensure that efficacy is maintained in in vitro assays of PTPRD and other PTP inhibitory activity. Bromo and ethynyl derivatives IA3-8Br and IA3-8C2H are already in our inventory (cf. FIG. 5); they are prepared as outlined in FIG. 23, top. Note that IA3-8Br is prepared using 1.1 equiv of ester 2, because 1.5 equiv of ester 2 in this case resulted in competing displacement of both bromides. Preliminary screening assays at 1 μM and 250 nM establish that these compounds are potent PTPRD inhibitors, on par with IA3 and close to illudalic acid (IA1, cf. FIG. 16).

Synthesis of Derivatized Probes

We develop the synthesis of functional probes by applying various manifestations of click chemistry to make five different biotin derivatives. First we propose to make azide IA3-8N3 by Cu₂O-mediated bromide-to-azide substitution⁷⁰ (FIG. 19, middle; alternatively, we could make IA3-8F9N3 by S_{N}Ar substitution of IA3-89F.) We expect IA3-8N3 (or IA3-8F9N3) to be a potent PTPRD inhibitor. Many biotinylating reagents for click couplings are available; we will start with tetra(ethylene glycol) (PEG4) linkers by analogy to our previous work.⁷¹ Sonogashira coupling of IA3-8Br with biotin-PEG4-alkyne produces IA3-Bt1. We expect the illudalic acid pharmacophore to be compatible with these couplings; Sonogashira and other Pd-catalyzed couplings are quite mild and can even be performed in vivo. Iodo-variant IA3-8I (not shown) is prepared from 2,5-diiodobenzoic acid by analogy to IA3-8Br if appropriate for Sonogashira applications.

We then use various azide-alkyne cycloaddition (AAC) protocols to prepare biotinylated illudalogs. Initial experiments are performed in convenient organic reaction media; then we validate the same coupling in HEPES buffer media as used in PTPRD screening assays (50 mM HEPES pH 7.5, but without protein and EDTA). We have experience developing reagents and applications of AAC couplings under copper-catalyzed (CuAAC) and/or strain-promoted (SPAAC) approaches. Here we start with alternative CuAAC couplings of IA3-8C2H and IA3-8N3 with biotin-PEG4-azide and alkyne, respectively, to make IA3-Bt2 and IA3-Bt3. These CuAAC couplings are suitable for in vitro and ex vivo (e.g., cell lysates) labeling applications but not for work in live cells because of copper toxicity. Biotin-PEG4-picolyl azide is better suited for CuAAC in live cells, because the picolyl azide ligates copper, which accelerates CuAAC and ameliorates copper-associated toxicity. SPAAC using biotin-PEG4-DBCO (cyclooctyne) or other biorthogonal reactions are developed for potential in vivo labeling experiments. Finally, we use similar chemistry to make fluorescent probes for bioimaging and proteolysis targeting chimeras (PROTACs) for myriad applications. We envision the covalent cysteine ligation unraveling after proteosomal degradation to enable catalytic PROTAC turnover; modified reversible inhibitors (cf. IA3-5Me, FIG. 21) may also be of use here.

We expect the proposed illudalog-based functional probes to support future PTPRD enzymology and pharmacology experiments in cells and potentially in vivo. We expect the illudalogs to be bioavailable by analogy to 7-BIA. Those persons of ordinary skill in the art will understand that alternative tethers and linkers may be examined as appropriate for specific future applications. We anticipate that the biotin conjugation chemistry will be effective for labeling cellular PTPRD and identifying off-target interactions. Any significant targets that emerge from cellular proteomics will be investigated and added to counter-screening panels for future rounds of inhibitor optimization.

This invention provides general and efficient synthetic access to potent PTPRD inhibitors as lead compounds for therapeutic development. Convergent benzannulation of β-keto amides (1) and esters (2), followed by a one-pot reduction/hydrolysis sequence, provides the polycyclic framework and trifunctional pharmacophore in only 2 steps, vs. ˜12 steps previously established to craft the pharmacophore (cf. FIG. 2). A 1-step annulation is also proposed for IA3. The concise synthetic approach enables rapid assembly of inhibitors and probes for future discovery.

### REFERENCES

1. Zhang Z Y. Drugging the Undruggable: Therapeutic Potential of Targeting Protein TyrosinePhosphatases. Acc Chem Res. 2017, 50, 122-129. PMID: 27977138; PMCID: PMC6347733.
2. (a) Stanford S M, Bottini N. Targeting Tyrosine Phosphatases: Time to End the Stigma. Trends Pharmacol Sci. 2017, 38, 524-540. PMID: 28412041 PMCID: PMC5494996. (b) Lazo J S, McQueeney K E, Sharlow E R. New Approaches to Difficult Drug Targets: The Phosphatase Story. SLAS Discov. 2017, 22, 1071-1083. PMID: 28745976. (c) Tautz L, Mustelin T. Strategies for developing protein tyrosine phosphatase inhibitors. Methods. 2007, 42, 250-260. PMID: 17532512.
3. (a) Mooney R A, LeVea C M. The leukocyte common antigen-related protein LAR: candidate PTP for inhibitory targeting. Curr Top Med Chem. 2003, 3, 809-819. PMID: 12678846. (b) Kulas D T, Goldstein B J, Mooney R A. The transmembrane protein-tyrosine phosphatase LAR modulates signaling by multiple receptor tyrosine kinases. J Biol Chem. 1996, 271, 748-754. PMID: 8557682.
4. Zabolotny J M, Kim Y B, Peroni O D, et al. Overexpression of the LAR (leukocyte antigen-related) protein-tyrosine phosphatase in muscle causes insulin resistance. Proc Natl Acad Sci USA. 2001, 98, 5187-5192. PMID: 11309481.
5. Chagnon M J, Uetani N, Tremblay M L. Functional significance of the LAR receptor protein tyrosine phosphatase family in development and diseases. Biochem Cell Biol. 2004, 82, 664-675. PMID: 15674434.
6. Ren, J.-M.; Li, P.-M.; Zhang, W.-R.; Sweet, L. J.; Cline, G.; Shulman, G. I.; Livingston, J. N.; Goldstein, B. J. Transgenic mice deficient in the LAR protein-type phosphatase exhibit profound defects in glucose homeostasis. Diabetes 1998, 47, 493-497. PMID: 9519761.
7. (a) Ahmad, F.; Considine, R. V.; Goldstein, B. J. Increased abundance of the receptor-type protein-tyrosine phosphatase LAR accounts for the elevated insulin receptor dephosphorylating activity in adipose tissue of obese human subjects. J. Clin. Ivest. 1995, 95, 2806-2812. PMID: 7769120 PMCID: PMC295966. (b) Miscio, G.; Tassi, V.; Coco, A.; Soccio, T., Di Paola, R.; Prudente, S.; Baratta, R.; Frittitta, L.; Ludovico, O.; Padovano, L.; Dallapiccola, B.; Di Mario, U.; De Cosmo, S.; Trischitta, V. The allelic variant of LAR gene promoter-127 bp T→A is associated with reduced risk of obesity and other features relatedto insulin resistance. J. Mol. Med. 2004, 82, 459-466. PMID: 15150650.
8. (a) Drgonova, J., Walther, D., Wang, K. J., Hartstein, G. L., Lochte, B., Troncoso, J., Uetani, N., Iwakura, Y., and Uhl, G. R. Mouse Model for Protein Tyrosine Phosphatase D (PTPRD) Associations with Restless Leg Syndrome or Willis-Ekbom Disease and Addiction: Reduced Expression Alters Locomotion, Sleep Behaviors and Cocaine-Conditioned Place Preference. Mol Med. 2015, 21, 717-725. PMID: 26181631. (b) Uhl G R, Martinez M J. PTPRD: neurobiology, genetics, and initial pharmacology of a pleiotropic contributor to brain phenotypes. Ann N Y Acad Sci. 2019, 1451, 112-129. PMID: 30648269.
9. (a) Levea C M, Mcgary C T, Symons J R, Mooney R A. PTP LAR expression compared to prognostic indices in metastatic and non-metastatic breast cancer. Breast Cancer Res Treat. 2000, 64, 221-228. PMID: 11194458. (b) Konishi, N., Tsujikawa, K., Yamamoto, H., Ishida, E., Nakamura, M., Shimada, K., Yane, K., Yamashita, H., and Noguchi, S. Overexpression of leucocyte common antigen (LAR) P-subunit in thyroid carcinomas. Br J Cancer. 2003, 88, 1223-1228. PMID: 12698188; PMCID: PMC2747580. (c) Lazo J S, McQueeney K E, Burnett J C, Wipf P, Sharlow E R. Small molecule targeting of PTPs in cancer. Int J Biochem Cell Biol. 2018, 96, 171-181. PMID: 28943273.
10. (a) Ohtake Y, Saito A, Li S. Diverse functions of protein tyrosine phosphatase a in the nervous and immune systems. Exp Neurol. 2018, 302, 196-204. PMID: 29374568. (b) Buchser W J, Slepak T I, Gutierrez-Arenas O, Bixby J L, Lemmon V P. Kinase/phosphatase overexpression reveals pathways regulating hippocampal neuron morphology. MolSyst Biol. 2010, 6, 391. PMID: 20664637 PMCID: PMC2925531. (c) Stepanek, L.; Stoker, A. W.; Stoeckli, E.; Bixby, J L. Receptor Tyrosine Phosphatases Guide Vertebrate Motor Axons during Development. J. Neurosci. 2005, 25, 3813-3823. PMID: 15829633 PMCID: PMC6724933. (d) Bernabeu R, Yang T, Xie Y, Mehta B, Ma S Y, Longo F M. Downregulation of the LAR protein tyrosine phosphatase receptor is associated with increased dentate gyrus neurogenesis and an increased number of granule cell layer neurons. Mol Cell Neurosci. 2006, 31, 723-738. PMID: 16488625.
11. (a) Whitty A. Growing PAINS in academic drug discovery. Future Med Chem. 2011, 3, 797-801. PMID: 21644825. (b) Baell J B, Holloway G A. New substructure filters for removal of pan assay interference compounds (PAINS) from screening libraries and for their exclusion in bioassays. J Med Chem. 2010, 53, 2719-2740. PMID: 20131845.
12. Yang, X.; Li, J.; Zhou, Y.; Shen, Q.; Chen, J.; Li, J. Discovery of novel inhibitor of human leukocyte common antigen-related phosphatase. Biochim. Biophys. Acta 2005, 1726, 34-41. PMID: 16198483.
13. Nair, M. S. R.; Takeshita, H.; McMorris, T. C.; Anchel, M. Metabolites of Clitocybe illudens. IV. Illudalic acid, a sesquiterpenoid, and illudinine, a sesquiterpenoid alkaloid. J. Org Chem. 1969, 34, 240-243. PMID: 5768225.
14. Uhl, G. R.; Martinez, M. J.; Paik, P.; Sulima, A.; Bi, G. H.; Iyer, M. R.; Gardner, E.; Rice, K. C.; Xi, Z. X. Cocaine reward is reduced by decreased expression of receptor-type protein tyrosine phosphatase D (PTPRD) and by a novel PTPRD antagonist. Proc. Natl. Acad. Sci. U.S.A. 2018, 15, 11597-11602. PMID: 30348770 PMCID: PMC6233130.
15. McCullough, B. S.; Batsomboon, P.; Hutchinson, K. B.; Dudley, G. B.; Barrios, A. M. Synthesis and PTP Inhibitory Activity of Illudalic Acid and Its Methyl Ether, with Insights into Selectivity for LAR PTP over Other Tyrosine Phosphatases Under Physiologically Relevant Conditions. J. Nat. Prod. 2019, 12, 3386-3393. PMID: 31809044.
16. Woodward, R. B.; Hoye, T. R. Total synthesis of illudinine, illudalic acid, and illudacetalic acid. J. Am. Chem. Soc. 1977, 99, 8007-8014.
17. Ling, Q.; Huang, Y.; Zhou, Y.; Cai, Z.; Xiong, B.; Zhang, Y.; Ma, L., Wang, X.; Li, X.; Li, J.; Shen, J. Illudalic acid as a potential LAR inhibitor: Synthesis, SAR, and preliminary studies on the mechanism of action. Bioorg. Med. Chem. 2008, 16, 7399-7409. PMID: 18579388.
18. Ling, Q., Zhou, Y.-Y., Cai, Z.-L., Zhang, Y-H., Xiong, B., Ma, L.-P., Wang, X., Li, X., Li, J., and Shen, J.-K. Synthesis and LAR inhibition of 7-alkoxy analogues of illudalic acid. Acta Pharmacol. Sin. 2010, 45, 1385-97. PMID: 21355526
19. Tonks N K. Protein tyrosine phosphatases—from housekeeping enzymes to master regulators of signal transduction. FEBS J. 2013, 280, 346-378. PMID: 23176256 PMCID: PMC3662559.
20. Alonso, A., Sasin, J., Bottini, N., Friedberg, I., Friedberg, I., Osterman, A., Godzik, A., Hunter, T., Dixon, J. and Mustelin, T., Protein tyrosine phosphatases in the human genome. Cell. 2004, 117, 699-711. PMID: 15186772.
21. (a) Tonks N K. Protein tyrosine phosphatases: from genes, to function, to disease. Nat Rev Mol Cell Biol. 2006, 7, 833-846. PMID: 17057753. (b) Cheng, A., Uetani, N., Lampron, C. and Tremblay, M. L., 2005. Protein tyrosine phosphatases as therapeutic targets. In Inhibitors of Protein Kinases and Protein Phosphatases (pp. 191-214). Springer, Berlin, Heidelberg.
22. (a) Tautz L, Pellecchia M, Mustelin T. Targeting the PTPome in human disease. Expert Opin Ther targets. 2006, 10, 157-177. PMID: 16441235. (b) Barr A J. Protein tyrosine phosphatases as drug targets: strategies and challenges of inhibitor development. Future Med Chem. 2010, 2, 1563-1576. PMID: 21426149. (c) Ran H, Tsutsumi R, Araki T, Neel B G. Sticking It to Cancer with Molecular Glue for SHP2. Cancer Cell. 2016, 30, 194-196. PMID: 27505669 PMCID: PMC5558882. (d) Chen Y N, LaMarche M J, Chan H M, Fekkes P, Garcia-Fortanet J, Acker M G, Antonakos B, Chen C H, Chen Z, Cooke V G, Dobson J R, Deng Z, Fei F, Firestone B, Fodor M, Fridrich C, Gao H, Grunenfelder D, Hao H X, Jacob J, Ho S, Hsiao K, Kang Z B, Karki R, Kato M, Larrow J, La Bonte L R, Lenoir F, Liu G, Liu S, Majumdar D, Meyer M J, Palermo M, Perez L, Pu M, Price E, Quinn C, Shakya S, Shultz M D, Slisz J, Venkatesan K, Wang P, Warmuth M, Williams S, Yang G, Yuan J, Zhang J H, Zhu P, Ramsey T, Keen N J, Sellers W R, Stams T, Fortin P D. Allosteric inhibition of SHP2 phosphatase inhibits cancers driven by receptor tyrosine kinases. Nature. 2016, 535, 148152. PMID: 27362227.
23. Zhao S, Sedwick D, Wang Z. Genetic alterations of protein tyrosine phosphatases in human cancers. Oncogene. 2015, 34, 3885-3894. PMID: 25263441.
24. (a) Cohen, P. Protein kinases—the major drug targets of the twenty-first century? Nat. Rev. Drug Discov. 2002, 1, 309-315. PMID: 12120282. (b) Gross, S.; Rahal, R.; Stransky, N.; Lengauer, C.; Hoeflich, K. P. Targeting cancer with kinase inhibitors. J. Clin. Invest. 2015, 125, 1780-1789. PMID: 25932675 PMCID: PMC4463189.
25. He, R.; Yu, Z.-H.; Zhang, R.; Zhang, Z.-Y. Protein tyrosine phosphatases as potential therapeutic targets. Acta Pharmacol. Sin. 2014, 35, 1227-1246. PMID: 25220640.
26. Dardaei L, Wang H Q, Singh M, Fordjour P, Shaw K X, Yoda S, Kerr G, Yu K, Liang J, Cao Y, Chen Y, Lawrence M S, Langenbucher A, Gainor J F, Friboulet L, Dagogo-Jack I, Myers D T, Labrot E, Ruddy D, Parks M, Lee D, DiCecca R H, Moody S, Hao H, Mohseni M, LaMarche M, Williams J, Hoffmaster K, Caponigro G, Shaw A T, Hata A N, Benes C H, Li F, Engelman J A. SHP2 inhibition restores sensitivity in ALK-rearranged non-small-cell lung cancer resistant to ALK inhibitors. Nat Med. 2018, 24, 512-517. PMID: 29505033 PMCID: PMC6343825.
27. Smith A M, Maguire-Nguyen K K, Rando T A, Zasloff M A, Strange K B, Yin V P. The protein tyrosine phosphatase 1B inhibitor MSI-1436 stimulates regeneration of heart and multiple other tissues. NPJ Regen Med. 2017, 2:4. doi:10.1038/s41536-017-0008-1. PMID: 29302341 PMCID: PMC5677970.
28. Nam, H.-J., Poy, F., Krueger, N. X., Saito, H., and Frederick, C. A. Crystal structure of the tandem phosphatase domains of RPTP LAR. Cell. 1999, 97, 449-457. PMID: 10338209.
29. (a) Speers A E, Adam G C, Cravatt B F. Activity-based protein profiling in vivo using a copper(I)-catalyzed azide-alkyne [3+2] cycloaddition. J Am Chem Soc. 2003, 125, 4686-4687. PMID: 12696868. (b) Speers A E, Cravatt B F. Profiling enzyme activities in vivo using click chemistry methods. Chem Biol. 2004, 11, 535-546. PMID: 15123248.
30. Xu, H.; Li, S.; Liu, H.; Fu, H.; Jiang, Y. Simple and efficient copper-catalyzed cascade synthesis of naphthols containing multifunctional groups under mild conditions. Chem. Commun. 2010, 46, 7617-7619. PMID: 20848012.
31. Anchel M, Hervey A, Robbins W J. Production of Illudin M and of aFourthCrystallineCompound by Clitocybe Illudens. Proc Natl Acad Sci USA. 1952, 38, 927-928. PMID: 16589202 PMCID: PMC1063684.
32. (a) Zhou L, Ma Y C, Tang X, Li W Y, Ma Y, Wang R L. Identification of the potential dual inhibitor of protein tyrosine phosphatase sigma and leukocyte common antigen-related phosphatase by virtual screen, molecular dynamic simulations and post-analysis [published online ahead of print, 2019 Dec. 27]. J Biomol Struct Dyn. 2019, 1-18. doi: 10.1080/07391102.2019.1705913. PMID: 31842717. (b) Wu J W, Zhang H, Li W Y, Tang X, Li H L, Lu X H, Zheng Z H, Ma Y, Wang R L. Design potential selective inhibitors for human leukocyte common antigen-related (PTP-LAR) with fragment replace approach [published online ahead of print, 2019 Dec. 9]. J Biomol Struct Dyn. 2019, 1-11. doi:10.1080/07391102.2019.1699862. PMID: 31787068. (c) Du S, Yang B, Wang X, Li W Y, Lu X H, Zheng Z H, Ma Y, Wang R L. Identification of potential leukocyte antigen-related protein (PTP-LAR) inhibitors through 3D QSAR pharmacophore-based virtual screening and molecular dynamics simulation [published online ahead of print, 2019 Oct. 17]. J Biomol Struct Dyn. 2019, 1-14. PMID: 31588870.
33. Kramer, N. J.; Hoang, T. T.; Dudley, G. B. Reaction discovery using neopentylene-tethered coupling partners: cycloisomerization/oxidation of electron-deficient dienynes. Org. Left. 2017, 19, 4636-4639. PMID: 28832157.
34. (a) Corey, E. J. and Cheng, X. M., The Logic of Chemical Synthesis. John Wiley & Sons, New York, 1991. (b) Corey, E. J. The logic of chemical synthesis: multistep synthesis of complex carbogenic molecules (Nobel Lecture). Angew. Chem. Int. Ed. 1991, 30, 455-465.
35. (a) Kamijo, S.; Dudley, G. B. Tandem nucleophilic addition/fragmentation reactions and synthetic versatility of vinylogous acyl triflates. J. Am. Chem. Soc. 2006, 128, 6499-6507. PMID: 16683816. (b) Tummatorn, J.; Dudley, G. B. Ring opening/fragmentation of dihydropyrones for the synthesis of homopropargyl alcohols. J. Am. Chem. Soc. 2008, 130, 5050-5051. PMID: 18357983. (c) Hoang, T. T.; Dudley, G. B. Synthesis of high-value 1,6-enynes by tandem fragmentation/olefination. Org. Lett. 2013, 15, 4026-4029. PMID: 23885858. (d) Yang, J.; Hoang, T. T.; Dudley, G. B. Alkynogenic fragmentation. Org. Chem. Front. 2019, 6, 2560-2569.
36. Hoang, T. T.; Birepinte, M.; Kramer, N. J.; Dudley, G. B. Six-step synthesis of alcyopterosin A, a bioactive illudalane sesquiterpene with a gem-dimethylcyclopentane ring. Org. Lett. 2016, 18, 3470-3473. PMID: 27359158.
37. (a) Morrison, A. E.; Hoang, T. T.; Birepinte, M.; Dudley, G. B. Synthesis of illudinine from dimedone. Org Lett. 2017, 19, 858-861. PMID: 28133968. (b) Gaston, R., Jr.; Geldenhuys, W. J.; Dudley, G. B. Synthesis of illudinine from dimedone and identification of activity as a monoamine oxidase inhibitor (MAOI). Manuscript submitted.
38. (a) Krishnamurthy D, Barrios A M. Profiling protein tyrosine phosphatase activity with mechanistic probes. Curr Opin Chem Biol. 2009, 13, 375-381. PMID: 19716756.
(b) Stanford, S. M., Krishnamurthy, D., Falk, M. D., Messina, R., Debnath, B., Li, S., Liu, T., Kazemi, R., Dahl, R., He, Y., Yu, X., Chan, A. C., Zhang, Z.-Y., Barrios, A. M., Woods, V. L. Jr., Neamati, N., and Bottini, N., Discovery of a novel series of inhibitors of lymphoid tyrosine phosphatase with activity in human T cells. J Med Chem. 2011, 54, 1640-1654. PMID: 21341673 PMCID: PMC3086468.
(c) Krishnamurthy, D., Karver, M. R., Fiorillo, E., Orru, V., Stanford, S. M., Bottini, N. and Barrios, A. M., Gold (I)-mediated inhibition of protein tyrosine phosphatases: a detailed in vitro and cellular study. J Med Chem. 2008, 51, 4790-4795. PMID: 18605719.
(d) Stanford, S. M., Krishnamurthy, D., Kulkarni, R. A., Karver, C. E., Bruenger, E., Walker, L. M., Ma, C. T., Chung, T. D., Sergienko, E., Bottini, N. and Barrios, A. M., pCAP-based peptide substrates: the new tool in the box of tyrosine phosphatase assays. Methods. 2014, 65, 165-174. PMID: 23886911 PMCID: PMC3899110.
(e) He, Y., Liu, S., Menon, A., Stanford, S., Oppong, E., Gunawan, A. M., Wu, L., Wu, D. J., Barrios, A. M., Bottini, N. and Cato, A. C., A potent and selective small-molecule inhibitor for the lymphoid-specific tyrosine phosphatase (LYP), a target associated with autoimmune diseases. J Med Chem. 2013, 56, 4990-5008. PMID: 23713581 PMCID: PMC3711248.
(f) Obiri, D. D., Flink, N., Maier, J. V., Neeb, A., Maddalo, D., Thiele, W., Menon, A., Stassen, M., Kulkarni, R. A., Garabedian, M. J. and Barrios, A. M., PEST-domain-enriched tyrosine phosphatase and glucocorticoids as regulators of anaphylaxis in mice. Allergy. 2012, 67, 175-182. PMID: 21981059.
(g) Stanford, S. M., Panchal, R. G., Walker, L. M., Wu, D. J., Falk, M. D., Mitra, S., Damle, S. S., Ruble, D., Kaltcheva, T., Zhang, S. Zhang, Z.-Y., Bavari, S., Barrios, A. M., and Bottini, N., High-throughput screen using a single-cell tyrosine phosphatase assayrevealsbiologicallyactive inhibitors of tyrosine phosphatase CD45. Proc Natl Acad Sci USA. 2012, 109, 13972-13977. PMID: 22891353 PMCID: PMC3435192.
(h) Biswas, S., McCullough, B. S., Ma, E. S., LaJoie, D., Russell, C. W., Garrett Brown, D., Round, J. L., Ullman, K. S., Mulvey, M. A., Barrios, A. M., Dual colorimetric and fluorogenic probes for visualizing tyrosine phosphatase activity. Chem Commun. 2017, 53, 2233-2236. PMID: 28144650.
39. Strelow J M. A Perspective on the Kinetics of Covalent and Irreversible Inhibition. SLAS Discov. 2017, 22, 3-20. PMID: 27703080.
40. Wender, P.; Quiroz, R.; Ho, S.; Shimizu, A.; Ryckbosch, S.; Stevens, M. C.; Jeffreys, M. S.; Hardman, C.; Sloane, J. Bryostatin compounds and methods of preparing the same. U.S. patent application Ser. No. 16/337,221, 2019.
41. Hanari, T.; Shimada, N.; Kurosaki, Y.; Thrimurtulu, N.; Nambu, H.; Anada, M.; Hashimoto, S. Asymmetric Total Sythesis of (−)-Englerin A through Catalytic Diastereo- and Enantioselective Carbonyl Ylide Cycloaddition. Chem.-Eur. J. 2015, 21, 11671-11676. PMID: 26179743.
42. Montalibet J, Skorey K I, Kennedy B P. Protein tyrosine phosphatase: enzymatic assays. Methods. 2005, 35, 2-8. PMID: 15588980.
43. Reijenga J, van Hoof A, van Loon A, Teunissen B. Development of Methods for the Determination of pKa Values. Anal Chem Insights. 2013, 8, 53-71. PMID: 23997574 PMCID: PMC3747999
44. (a) Ahmed V F, Bottini N, Barrios A M. Covalent inhibition of the lymphoid tyrosine phosphatase. ChemMedChem. 2014, 9, 296-299. PMID: 24403103. (b) Liu S, Zhou B, Yang H, He Y, Jiang Z X, Kumar S, Wu L, Zhang Z Y. Aryl vinyl sulfonates and sulfones as active site-directed and mechanism-based probes for protein tyrosine phosphatases. J Am Chem Soc. 2008, 130, 8251-8260. PMID: 18528979 PMCID: PMC2810288.
45. Robart, A. R.; Chan, R. T.; Peters, J. K.; Rajashankar, K. R.; Toor, N. Crystal structure of a eukaryotic group II intron lariat. Nature 2014, 514, 193-197. PMID: 25252982.
46. https://www.aps.anl.gov/
47. https://www.rcsb.org/
48. Almo, S. C., Bonanno, J. B., Sauder, J. M., Emtage, S., Dilorenzo, T. P., Malashkevich, V., Wasserman, S. R., Swaminathan, S., Eswaramoorthy, S., Agarwal, R. and Kumaran, D., Structural genomics of protein phosphatases. J Struct Funct Genomics. 2007, 8, 121140. PMID: 18058037.
49. (a) Peyser, N. D., Du, Y., Li, H., Lui, V., Xiao, X., Chan, T. A. and Grandis, J. R., Loss-of-Function PTPRD Mutations Lead to Increased STAT3 Activation and Sensitivity to STAT3 Inhibition in Head and Neck Cancer. PLoS One. 2015, 10, e0135750. PMID: 26267899 PMCID: PMC4534317. (b) Kim M, Morales L D, Jang I S, Cho Y Y, Kim D J. Protein Tyrosine Phosphatases as Potential Regulators of STAT3 Signaling. Int. J Mol Sci. 2018, 19, 2708. PMID: 30208623 PMCID: PMC6164089. 2708. (c) Veeriah, S., Brennan, C., Meng, S., Singh, B., Fagin, J. A., Solit, D. B., Paty, P. B., Rohle, D., Vivanco, I., Chmielecki, J. and Pao, W., The tyrosine phosphatase PTPRD is a tumor suppressor that is frequently inactivated and mutated in glioblastoma and other human cancers. Proc Natl Acad Sci USA. 2009, 106, 9435-9440. PMID: 19478061 PMCID: PMC2687998.
50. Yang T, Zhang J S, Massa S M, Han X, Longo F M. Leukocyte common antigen-related tyrosine phosphatase receptor: increased expression and neuronal-type splicing in breast cancer cells and tissue. Mol Carcinog. 1999, 25, 139-149. PMID: 10365916.
51. Kypta R M, Su H, Reichardt L F. Association between a transmembrane protein tyrosine phosphatase and the cadherin-catenin complex. J Cell Biol. 1996, 134, 1519-1529. PMID: 8830779 PMCID: PMC2121007.
52. Parker C G, Pratt M R. Click Chemistry in Proteomic Investigations. Cell. 2020, 180, 605-632. PMID: 32059777.
53. Otto J F, Kimball M M, Wilcox K S. Effects of the anticonvulsant retigabine on cultured cortical neurons: changes in electroresponsive properties and synaptic transmission. Mol Pharmacol. 2002, 61, 921-927. PMID: 11901232.
54. Geissler M, Gottschling C, Aguado A, Rauch U, Wetzel C H, Hatt H, Faissner A. Primary hippocampal neurons, which lack four crucial extracellular matrix molecules, display abnormalities of synaptic structure and function and severe deficits in perineuronal net formation. J Neurosci. 2013, 33, 7742-7755. PMID: 23637166 PMCID: PMC6618965.
55. Rizkallah, R., Batsomboon, P.; Dudley, G. B.; Hurt, M. The Oncogenic Kinase TOPK/PBK is a Master Mitotic Regulator of C2H2 Zinc Finger Proteins. Oncotarget 2015, 6, 1446-1461. PMID: 25575812 PMCID: PMC4359306.
56. Hajipour, A. R. and Mohammadsaleh, F., Synthesis of aryl azides from aryl halides promoted by Cu₂O/tetraethylammonium prolinate. Tetrahedron Lett. 2014, 55, 6799-6802.
57. (a) Li N, Lim R K, Edwardraja S, Lin Q. Copper-free Sonogashira cross-coupling for functionalization of alkyne-encoded proteins in aqueous medium and in bacterial cells. J Am Chem Soc. 2011, 133, 15316-15319. PMID: 21899368 PMCID: PMC3184007. (b) Chankeshwara S V, Indrigo E, Bradley M. Palladium-mediated chemistry in living cells. Curr Opin Chem Biol. 2014, 21, 128-135. PMID: 25108198.
58. Page, J. P.; Robinson, J. W.; Albrecht, K. O.; Cosimbescu, L. Synthesis of a biofuel target through conventional organic chemistry. Tetrahedron Lett. 2018, 59, 1421-1423.
59. Stork, G.; Danheiser, R. L. Regiospecific alkylation of cyclic p-diketone enol ethers. General synthesis of 4-alkylcyclohexenones. J. Org. Chem. 1973, 38, 1775-1776.
60. Crabtree, S. R.; Chu, W. A.; Mander, L. N. C-acylation of enolates by methyl cyanoformate: An examination of site-and stereoselectivity. Synlett 1990, 169-170.

The present invention provides an efficient synthetic access to potent PTPRD inhibitors as lead compounds for therapeutic use. The process of this invention provides convergent benzannulation of β-keto amides and esters, followed by a one-pot reduction/hydrolysis sequence, provides the polycyclic framework and trifunctional pharmacophore in only 2 steps versus the more than 12 steps of the background art. A one step annulation is also provided for compound IA3. The concise synthetic approach provided by this invention enables rapid assembly of the illudalog compounds of this invention, as set forth herein, and that are potent PTPRD inhibitors.

It will be appreciated by those persons skilled in the art that changes could be made to embodiments of the present invention described herein without departing from the broad inventive concept thereof. It is understood, therefore, that this invention is not limited by any particular embodiments disclosed, but is intended to cover the modifications that are within the spirit and scope of the invention, as defined by the appended claims.

What is claimed is:

1. A method of synthesizing illudalic acid comprising carrying out each of the five steps of the following scheme:

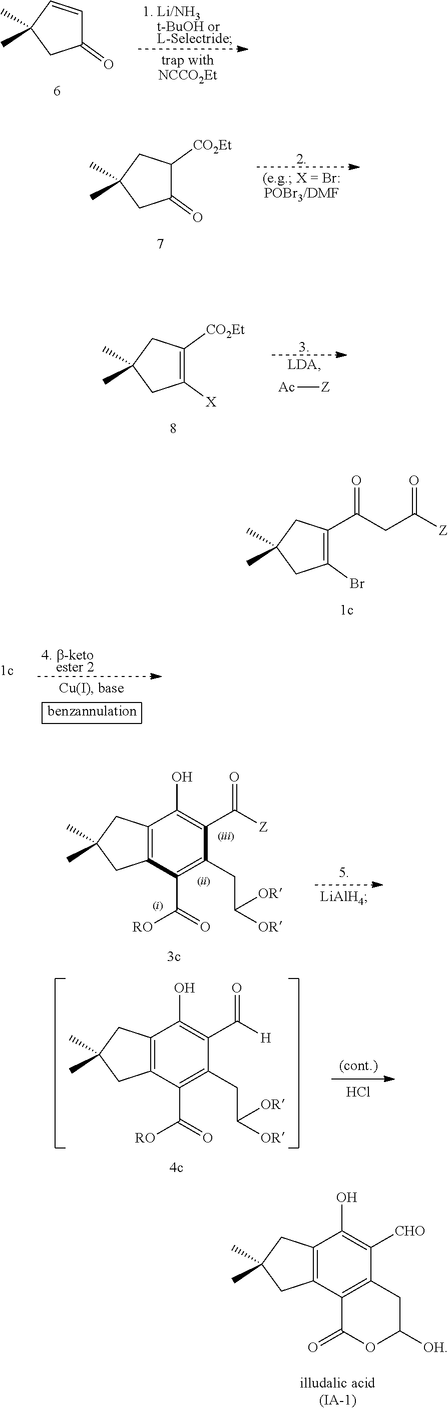

2. A method of preparing illudalic acid comprising:
providing benzannulation of a β-keto amide having a formula 3a 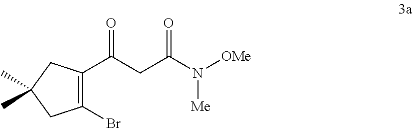
with a β-keto ester having a formula 4 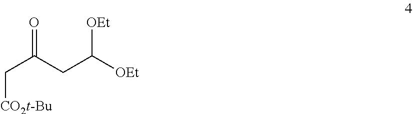 [SMILES: CCOC(CC(=O)CC(=O)OC(C)(C)C)OCC]
to produce a precursor compound having a formula 9a 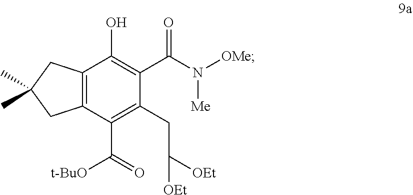
carrying out reduction of said precursor compound 9a with LiAlH₄ to form a reduced precursor compound having a formula 12 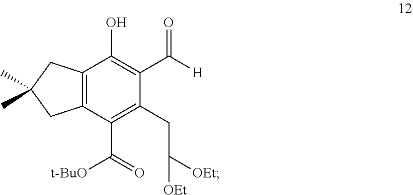
and
carrying out acid hydrolysis of said reduced precursor compound 12 to form illudalic acid having a formula IA1

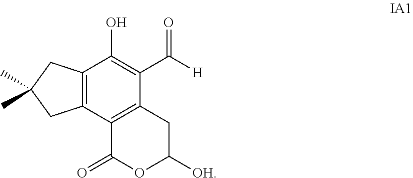

3. The method of claim 2 including subjecting a tetralin compound analogous to said reduced precursor compound 12 to two equivalents of LiAlH₄ to form a tetralin compound having a formula IA2

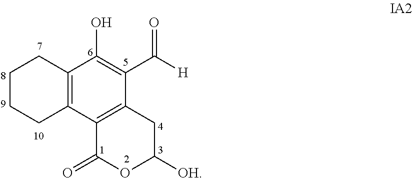 [SMILES: O=Cc1c(O)c2c(c3c1CC(O)OC3=O)CCCC2]

4. The method of claim 2 including subjecting a naphthalene compound analogous to said reduced precursor compound 12 to two equivalents of LiAlH₄ to form a naphthalene compound having a formula IA3

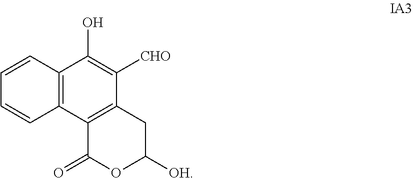 [SMILES: O=Cc1c2c(c3ccccc3c1O)C(=O)OC(O)C2]

5. A compound having a formula 13:

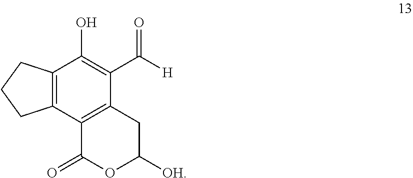 [SMILES: O=Cc1c(O)c2c(c3c1CC(O)OC3=O)CCC2]

6. A compound having a formula 14:

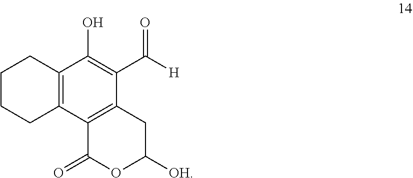 [SMILES: O=Cc1c(O)c2c(c3c1CC(O)OC3=O)CCCC2]

7. A compound having a formula 15:

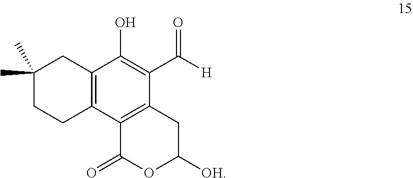

8. A compound of a formula 16:

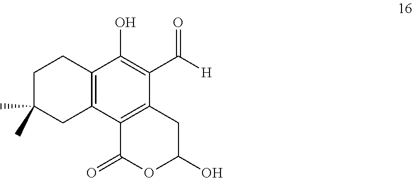

9. A compound that is one selected from the group of compounds consisting of IA1-6OMe, IA1-8H2, and IA1-6OMe-8H2, having one of a formula:

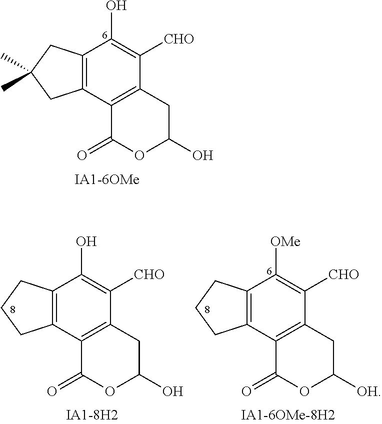

10. A compound that is one selected from the group of compounds consisting of IA2, IA2-8Me2, and IA2-9Me2, having one of a formula:

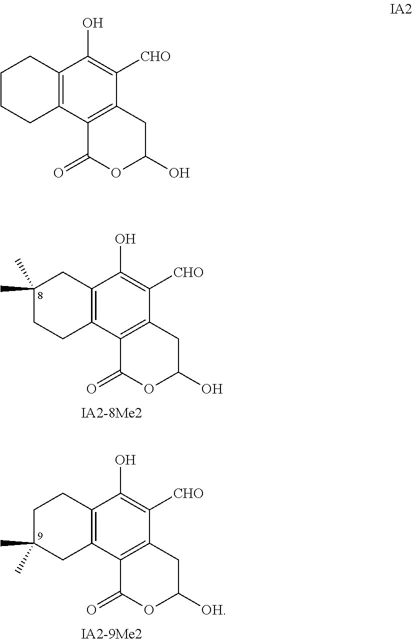

11. A compound that is one selected from the group of compounds consisting of IA3, IA3-6OMe, IA3-6OEt, IA3-6OC3H3, IA3-6OBn, IA3-7Me, IA3-8Me, IA3-9Me, IA3-8Br, IA3-8C2H, IA3-8Cl, IA3-9C1, IA3-9tBu, IA3-9CF3, IA3-7OMe, IA3-8OMe, IA3-89OMe, IA3-89F, and IA3-6OMe89F, having one of a formula:

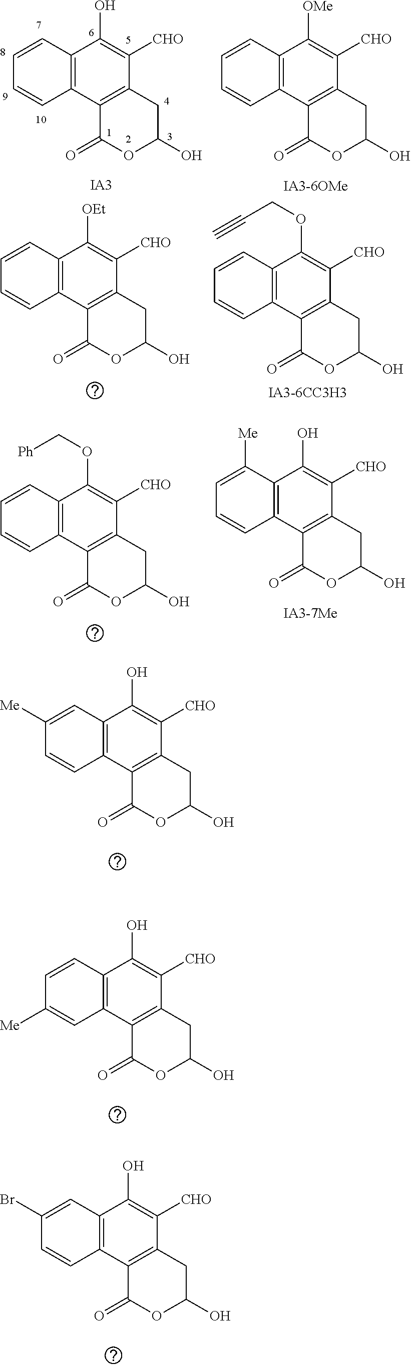

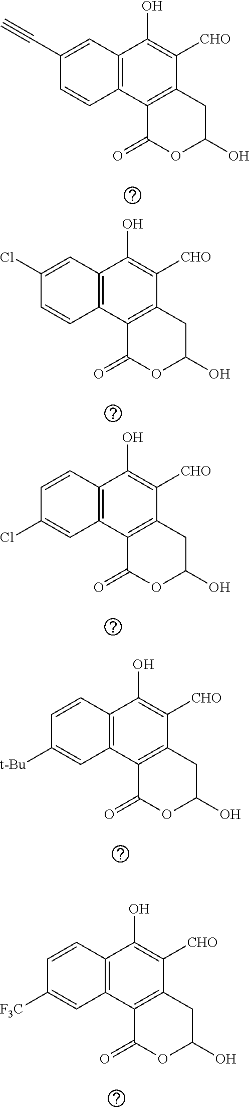 [SMILES: C#Cc1ccc2c3c(c(C=O)c(O)c2c1)CC(O)OC3=O ; O=Cc1c2c(c3ccc(Cl)cc3c1O)C(=O)OC(O)C2 ; O=Cc1c2c(c3cc(Cl)ccc3c1O)C(=O)OC(O)C2 ; CC(C)(C)c1ccc2c(O)c(C=O)c3c(c2c1)C(=O)OC(O)C3 ; O=Cc1c2c(c3cc(C(F)(F)F)ccc3c1O)C(=O)OC(O)C2]

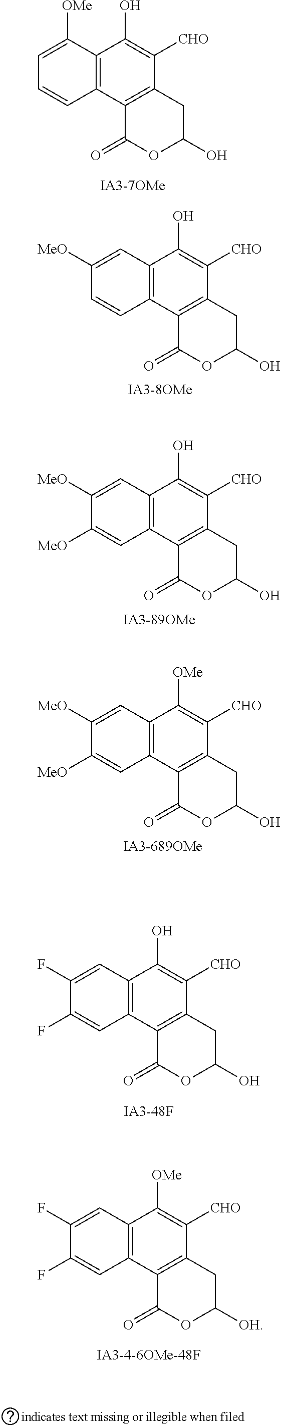

12. A compound that is one selected from the group of compounds consisting of: IA-5, IA-6, IA-7, IA-8, IA-9, and IA-10, having one of a formula: 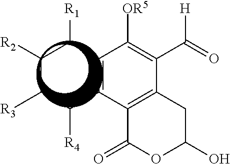
IA-5: R₁, R₂, R₃, and R₄═H, and R⁵═H
IA-6: R₁, R₂, R₃, and R₄═H, and R⁵=Me (i.e. CH₃)
IA-7: R₁ and R₄═H, and R₂ and R₃=MeO, and R⁵═H
IA-8: R₁, R₃, and R₄═H, and R₂═Cl, and R⁵═H
IA-9: R₁, R₂, and R₄═H, and R₃═Cl, and R⁵═H
IA-10: R₁, R₂, and R₄═H, and R₃=t-Bu, and R⁵═H.

13. A compound of a formula IA-4: 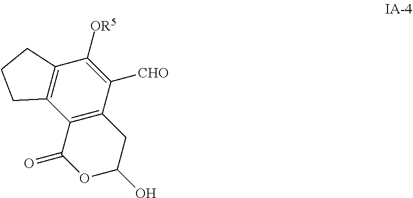
wherein R⁵ is a methyl group.

14. A compound that is one selected from the group of compounds consisting of IA-11, IA-12, IA-13, IA-14, IA-15, IA-16, IA-17, IA-18, IA-19, IA-20, IA-21, IA-22, IA-23, IA-24, IA-25, IA-26, 1-27, IA-28, IA-29, and IA-30, having one of a formula: 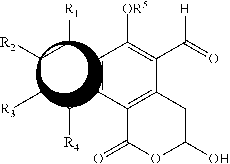
IA-11: Rⱼ=Me, R⁵═H, and R₂— R₄═H
IA-12: R₃=Me, R⁵=Me, and R₂—R₄═H
IA-13: R₂=Me, R⁵═H, and R₁, R₃, and R₄═H
IA-14 R₂=Me, R⁵=Me, and R₁, R₃, and R₄═H
IA-15: R₃=Me, R⁵═H, and R₁, R₂, and R₄═H
IA-16: R₃=Me, R⁵=Me, and R₁, R₂, and R₄═H
IA-17: R₄=Me, R⁵═H, and R₁—R₃═H
IA-18: R₄=Me, R⁵=Me, and R₁—R₃═H
IA-19: R₂═Br, R⁵═H, and R₁, R₃, and R₄═H
IA-20: R₂═Br, R⁵=Me, and R₁, R₃, and R₄═H
IA-21: R₁═OMe, R⁵═H, and R₂—R₄═H
IA-22: R₁═OMe, R⁵=Me, and R₂—R₄═H
IA-23: R₂═OMe, R⁵═H, and R₁, R₃, and R₄═H
IA-24: R₂═OMe, R⁵=Me, and R₁, R₃, and R₄═H
IA-25: R₃═OMe, R⁵═H, and R₁, R₂, and R₄═H
IA-26: R₃═OMe, R⁵=Me, and R₁, R₂, and R₄═H
IA-27: R₄═OMe, R⁵═H, and R₁—R₃═H
IA-28: R₄═OMe, R⁵=Me, and R₁—R₃═H
IA-29: R₁—R₃═F, R⁵═H, and R₄═H
IA-30: R₁—R₃═F, R⁵=Me, and R₄═H.

15. A compound that is one selected from the group of compounds consisting of IA-31, IA-32, IA-33, IA-34, IA-35, IA-36, IA-37, IA-38, IA-39, IA-40, IA-41, IA-42, IA-43, IA-44, IA-45, IA-46, IA-47, IA-48, IA-49, IA-50, IA-51, IA-52, IA-53, IA-54, and IA-55, having one of a formula:

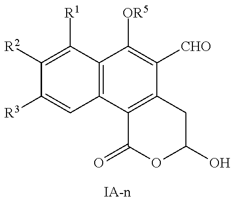

| | R¹⁻³ = H | R^{2,3} = OMe | R¹⁻³ = F | R¹ = Me | R² = Br |
|---|---|---|---|---|---|
| R⁵ = ethyl | IA-31 | IA-36 | IA-41 | IA-46 | IA-51 |
| R⁵ = butyl | IA-32 | IA-37 | IA-42 | IA-47 | IA-52 |
| R⁵ = benzyl | IA-33 | IA-38 | IA-43 | IA-48 | IA-53 |
| R⁵ = propargyl | IA-34 | IA-39 | IA-44 | IA-49 | IA-54 |
| R⁵ = CH₂CO₂Et | IA-35 | IA-40 | IA-45 | IA-50 | IA-55 |

wherein IA-36-IA-40: R₁═H; wherein IA-46-IA-50: R₂ and R₃═H; and wherein IA-51-IA 55: R₁ and R₃═H.

16. A compound that is one selected from the group of compounds consisting of IA-105-IA108, having one of a formula: 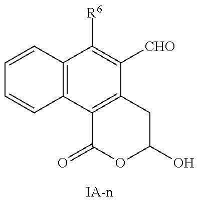
IA-105: R⁶ = methyl
IA-106: R⁶ = ethynyl
IA-107: R⁶ = allyl
IA-108: R⁶ = phenyl
IA-109: R⁶ = 2-furyl
IA-110: R⁶ = H

17. A compound that is one selected from the group of compounds consisting of IA3-5Me, IA3-5OMe, IA3-3H, and IA3-1Me, having one of a formula:

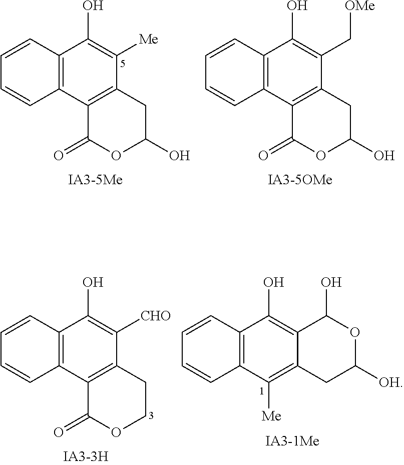

18. A compound that is one selected from the group of compounds consisting of IA-19, and IA-91-IA-97, having one of a formula:

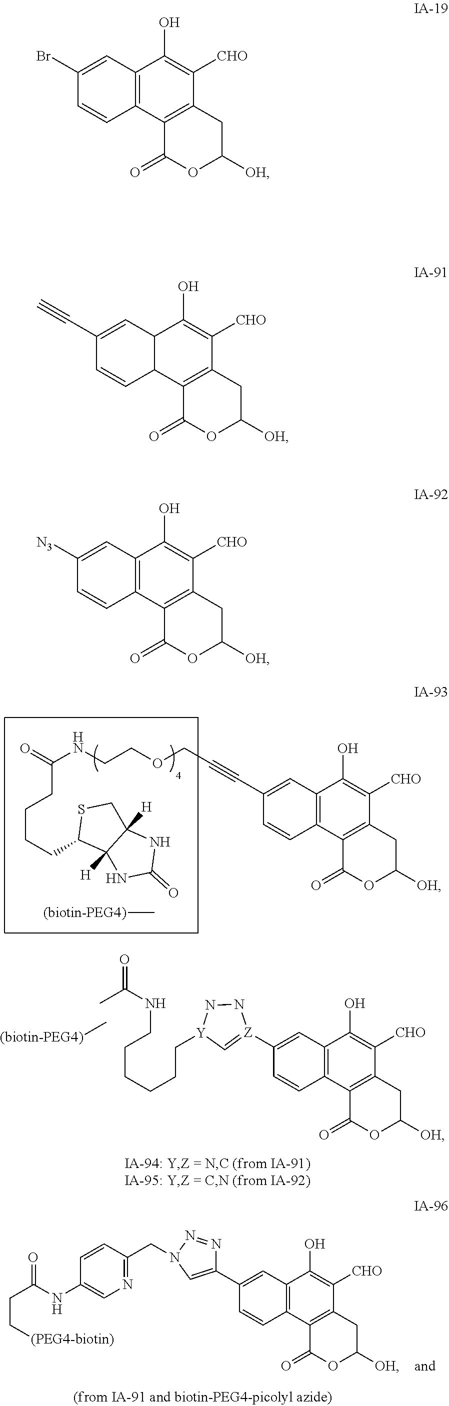

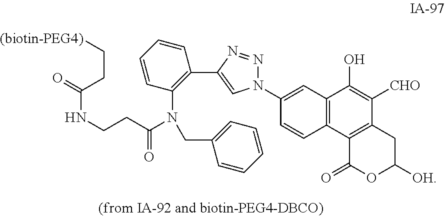

19. A compound that is one selected from the group of compounds consisting of one of the following structures: [chemical structure]
